(19) United States
(12) Patent Application Publication
(10) Pub. No.: US 2005/0287596 A9
(43) Pub. Date: Dec. 29, 2005

(54) Novel ligands and libraries of ligands

(76) Inventors: Andrew C. Braisted, San Francisco, CA (US); Daniel A. Erlanson, San Francisco, CA (US); Jeffrey W. Jacobs, San Mateo, CA (US)

Correspondence Address: CHOATE, HALL & STEWART LLP, TWO INTERNATIONAL PLACE, BOSTON, MA, 02110, US

(21) Appl. No.: 10/143,455

(22) Filed: May 10, 2002

Related U.S. Application Data
(63) Continuation-in-part of application No. 10/121,216, filed on Apr. 10, 2002.
Application No. 10/121,216 is a continuation-in-part of application No. 09/981,547, filed on Oct. 17, 2001.
Application No. 09/981,547 is a division of application No. 09/105,372, filed on Jun. 26, 1998, now Pat. No. 6,335,155.
Application No. 09/981,547 is a continuation-in-part of application No. 09/990,421, filed on Nov. 21, 2001, now Pat. No. 6,919,178.
(60) Provisional application No. 60/252,294, filed on Nov. 21, 2000.
(60) Provisional application No. 60/310,725, filed on Aug. 7, 2001.

(51) Int. Cl. C12Q 1/00; G01N 33/53; G01N 33/543; C07C 321/00; C07C 323/00; C07C 381/00; C07C 333/00

(52) U.S. Cl. 435/7.1; 436/518; 558/232; 560/157; 560/24; 564/32; 564/162; 564/85

(57) ABSTRACT

The present invention provides compounds and libraries of compounds having formula (I): 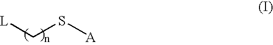 wherein L, n, S and A are defined generally and subsets herein. These compounds and libraries of compounds are useful generally in the drug discovery process.

### PRIORITY INFORMATION

This application is a continuation-in-part of U.S. Ser. No. 10/121,216 filed Apr. 10, 2002. The '216 application is a continuation-in-part of U.S. Ser. No. 09/981,547 filed Oct. 17, 2001 which is a divisional of U.S. Ser. No. 09/105,372 filed Jun. 26, 1998, and is a continuation-in-part of U.S. Ser. No. 09/990,421 filed Nov. 21, 2001 which asserts priority to U.S. Provisional Application No. 60/252,294 filed Nov. 21, 2000. All of these priority applications are incorporated herein by reference.

### BACKGROUND OF THE INVENTION

In general, the drug discovery process begins with the screening of a large number of compounds to identify modest affinity leads (K^{d}˜1 to 10 μM). An important tool in this process is the use of combinatorial libraries. Specifically, combinatorial methods for the generation of small molecule libraries and subsequent screening en masse have become important technologies for the identification of small molecule ligands to biological macromolecules (see, for example, Thompson et al. Chem. Rev. 1996, 96, 555-600; Balkenhohel et al. Angew. Chem. Int. Ed. Engl. 1996, 35, 2288-2337; Dolle, R. E. Mol. Diversity 1998, 3, 199-233; and Dolle et al. J. Comb. Chem. 1999, 1, 235-282).

Clearly, the ligands that are identified using this process serve as powerful tools for pharmacological studies and for drug development. The most successful libraries to date have been those based upon specific information such as knowledge of the mechanism or structure of the biological target, or by basing the library upon lead compounds that have been previously identified to bind to a target (see, for example, Kick et al. Chem. Biol. 1997, 4, 297-307; Rockwell et al. J. Am. Chem. Soc. 1996, 118, 10337-10338; Gray et al. Science 1998, 281, 533-538; Yang et al. Proc. Natl. Acad. Sci. USA 1998, 95, 10836-10841; Rohrer et al. Science 1998, 282, 737-740).

Unfortunately, although some targets are well suited for this screening process, most are problematic because moderate affinity leads are difficult to obtain. Identifying and subsequently optimizing weaker binding compounds would improve the success rate, but this would necessitate screening at higher concentrations and screening at high concentrations is generally impractical because of compound insolubility and assay artifacts. Moreover, the typical screening process does not target specific sites for drug design, only those sites for which a high-throughput assay is available. Finally, many traditional screening methods rely on inhibition assays that are often subject to artifacts caused by reactive chemical species or denaturants.

Erlanson et al., Proc. Nat. Acad Sci. USA 2000, 97,9367-9372, have recently reported a new strategy, called “tethering”, to rapidly and reliably identify small (˜250 Da) soluble drug fragments that bind with low affinity to a specifically targeted site on a protein or other macromolecule, using an intermediary disulfide “tether.” According to this approach, a library of disulfide-containing molecules is allowed to react with a cysteine-containing target protein under partially reducing conditions that promote rapid thiol exchange. If a molecule has even weak affinity for the target protein, the disulfide bond (“tether”) linking the molecule to the target protein will be entropically stabilized. The disulfide-tethered fragments can then be identified by a variety of methods, including mass spectrometry (MS), and their affinity improved by traditional approaches upon removal of the disulfide tether. See also PCT Publication No. WO 00/00823, published on Jan. 6, 2000 and U.S. Pat. No. 6,335,155.

So that the potential of the tethering method can be more fully realized, there remains a need to expand upon the libraries of compounds that are amenable for use with this approach. Among other things, the present invention provides such libraries.

### DESCRIPTION OF THE FIGURES

FIG. 1 schematically illustrates one embodiment of the tethering method.

FIG. 2A depicts the deconvoluted mass spectrum of the reaction of TS with a pool of 110 different ligand candidates with little or no binding affinity for TS.

FIG. 2B depicts the deconvoluted mass spectrum of the reaction of TS with a pool of 10 different ligand candidates where one of the ligand candidates possesses an inherent binding affinity to the enzyme.

FIG. 3 depicts three experiments where TS is reacted with the same library pool containing the selected N-tosyl D-proline compound in the presence of increasing concentration of the reducing agent, 2-mercaptoethanol.

FIG. 4 depicts schematically how tethering is utilized to identify a binding determinant.

FIG. 5 depicts schematically a method where two separate tethering experiments are used to identify binding determinants that are subsequently linked together to form a conjugate molecule that binds to the target protein.

FIG. 6 illustrates one embodiment of the tethering method using extenders.

### DESCRIPTION OF THE INVENTION

As described above, there remains a need to accelerate the drug discovery process. In general, the present invention expands upon the general tethering approach described above and provides novel compounds and libraries of compounds for use in this approach. Specifically, the novel compounds and libraries described herein provide powerful tools for the development of drug leads, and are useful for the identification of fragments that bind weakly, or with moderate binding affinity, to a biological target site of interest.

1) General Description of Compounds and Libraries of the Invention

The compounds of the invention include compounds and libraries of the general formula (I) as further defined below: 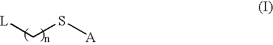
wherein A is —S(CH₂)ₚR^{A1} or —S(O)₂R^{A2}, wherein p is 1-5, R^{A1} is —NR^{A3}R^{A4}; OR^{A3}; SR^{A3}; —NHCOR^{A3}; —NHCONR^{A3}R^{A4}; —NR^{A3}R^{A4}R^{A5+}X⁻, wherein X is a halogen; —COOR^{A3}; CONR^{A4}R^{A4}; —SO₃R^{A3}; —OPO₃R^{A3}; —SO₂R^{A3}; and wherein R^{A2} is an aliphatic, heteroaliphatic, aryl, or heteroaryl moiety, and each occurrence of R^{A3}, R^{A4}, and R^{A5} is independently hydrogen, a protecting group, or an aliphatic, heteroaliphatic, aryl or heteroaryl moiety;
n is 0-5;
L is a moiety having one of the structures: 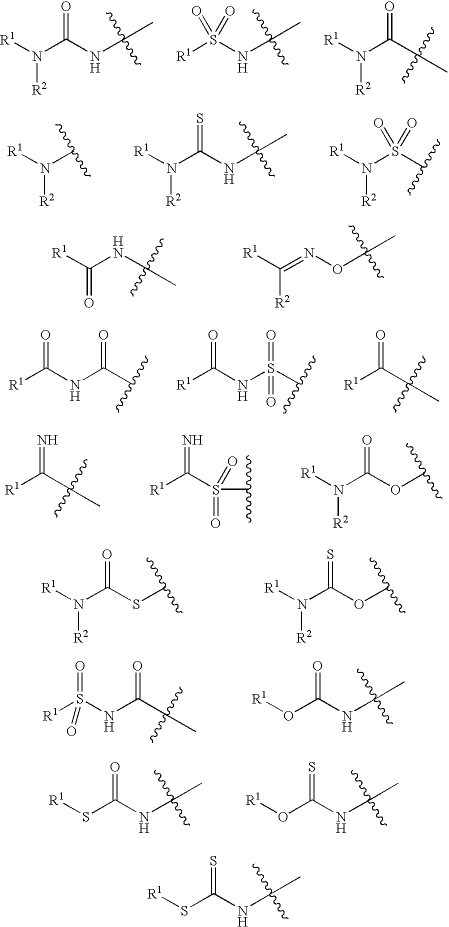
each occurrence of R¹ and R² is independently hydrogen, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, or wherein R¹ and R² taken together are a cycloaliphatic, heterocycloaliphatic, aryl or heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

It will be appreciated that for compounds and libraries as generally described above, certain classes of compounds and libraries of special interest include those in which L is one of the following structures: 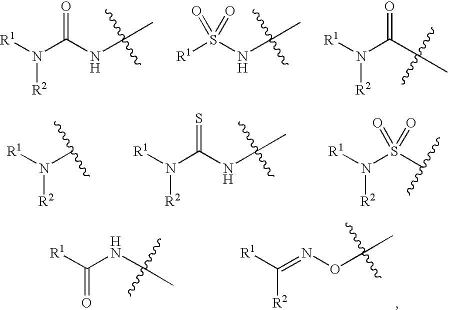
wherein R¹ and R² are each described generally above and in exemplary embodiments herein.

In certain other embodiments, compounds and libraries of special interest include those compounds and libraries wherein 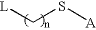
represents one of the structures: 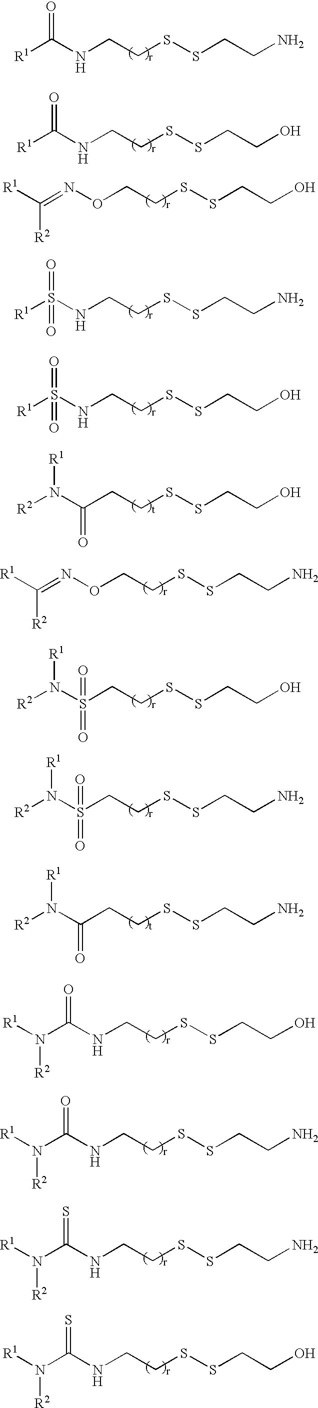
wherein r is 1 or 2; and t is 0, 1 or 2.

In certain other embodiments, compounds and libraries of special interest include those compounds and libraries wherein 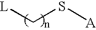
represents one of the structures: 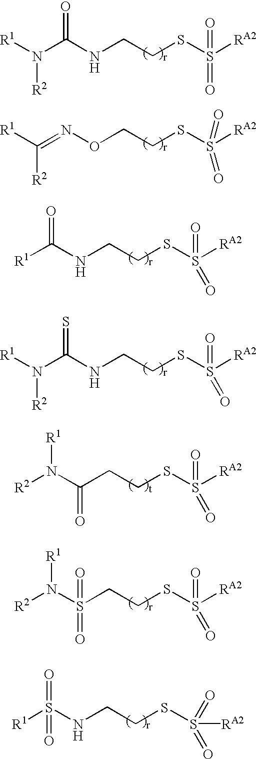
wherein r is 1 or 2; and R^{A2} is an alkyl, heteroalkyl, aryl, heteroaryl, -(alkyl)aryl, -(alkyl)heteroaryl, -(heteroalkyl)aryl, or -(heteroalkyl)heteroaryl moiety.

In certain embodiments of special interest for the compounds described directly above, R^{A2} is methyl or phenyl.

In yet other embodiments, certain classes of compounds and libraries of special interest include those compounds and libraries in which R¹ or R² is 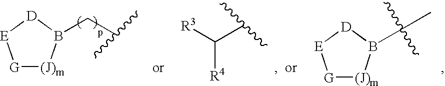
wherein R¹ and R² taken together form a cyclic moiety having the structure:
wherein B—D, D—E, E—G, G—J, two or more occurrences of J, and J—B are each independently joined by a single or double bond as valency and stability permit, wherein B is N, CH or C, D is —NR^{D}—, ═N—, —O—, —CHR^{D}—, or ═CR^{D}—, E is —NR^{E}—, ═N—, —O—, —CHR^{E}—-, or ═CR^{E}—, G is —NR^{G}—, ═N—, —O—, —CHR^{G}—, or ═CR^{G}—, each occurrence of J is independently —NR^{J}—, ═N—, —O—, —CHR^{J}—, or ═CR^{J}—,
m is 0-4and p is 0-4,
each occurrence of R³, R⁴, R^{D}, R^{E}, R^{G} and R^{J} is independently hydrogen, a protecting group, —(CR⁷R⁸)_{q}NR⁵R⁶, —(CR⁷R⁸)_{q}OR⁵, —(CR⁷R⁸)_{q}SR⁵, —(CR⁷R⁸)_{q}(C═O)R⁵, —(CR⁷R⁸)_{q}(C═O)OR⁵; —(CR⁷R⁸)_{q}(C═O)NR⁵R⁶, —(CR⁷R⁸)_{q}S(O)₂R⁵, —(CR⁷R⁵)_{q}NR⁵(C═O)R⁶, —(CR⁷R⁸)_{q}NR⁵(C═O)OR⁶, —(CR⁷R⁸)_{q}S(O)₂NR⁵R⁶, —(CR⁷R⁸)_{q}NR⁵S(O)₂R⁶, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
q is 0-4; and
each occurrence of R⁵, R⁶, R⁷ and R⁸ is independently hydrogen, a protecting group, or an aliphatic, heteroalipahtic, aryl, heteroaryl, -(aliphatic)aryl, -(aliphatic)heteroaryl, -(heteroalipahtic)aryl, or -(heteroaliphatic)heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

In still other embodiments, certain classes of compounds and libraries of special interest include those compounds and libraries in which 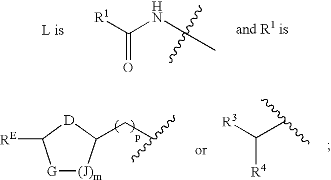
wherein m is 0-4, p is 0-4, D is CHR^{D} or NR^{D}, G is CHR^{G} or NR^{G}, and each occurrence of J is independently CHR^{J} or NR^{J}, wherein each occurrence of R^{D}, R^{E}, R^{G}, R^{J}, R³, and R⁴ is independently hydrogen, a protecting group, —(CR⁷R⁸)_{q}NR⁵R⁶, —(CR⁷R⁸)_{q}OR⁵, —(CR⁷R⁸)_{q}SR, —(CR⁷R⁸)_{q}(C═O)R⁵, —(CR⁷R⁸)_{q}(C═O)NR⁵R⁶ (CR⁷R⁸)_{q}S(O)₂R⁵, —(CR⁷R⁸)_{q}NR⁵(C═O)R⁶, —(CR⁷R⁸)_{q}S(O)₂NR⁵R⁶, —(CR⁷R⁸)_{q}NR⁵S(O)₂R⁶, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, wherein q is 0-4; and wherein each occurrence of R⁵, R⁶, R⁷ and R⁸ is independently hydrogen, a protecting group, or an aliphatic, heteroalipahtic, aryl, heteroaryl, -(aliphatic)aryl, -(aliphatic)heteroaryl, -(heteroalipahtic)aryl, or -(heteroaliphatic)heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

In yet other embodiments, certain classes of compounds and libraries of special interest include those compounds and libraries in which L is 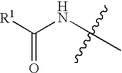
and R¹ is one of the structures:

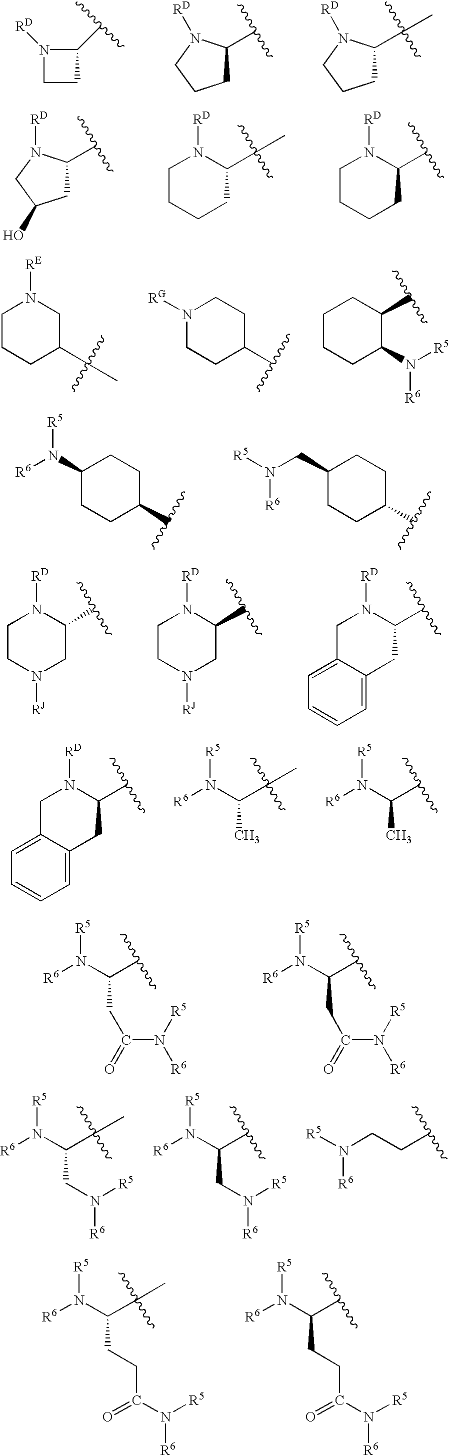

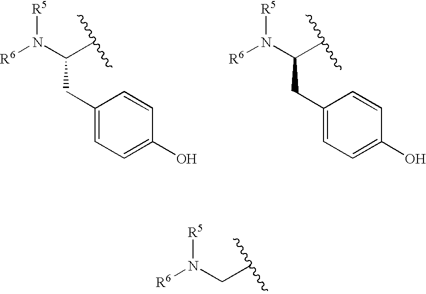

In still other embodiments, compounds and libraries of special interest include those compounds and libraries as generally described above, in which L is 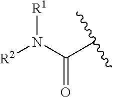
and one or both of R¹ and R² is 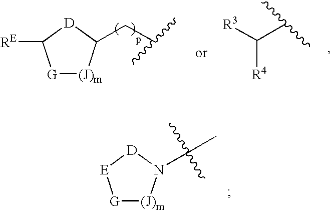
or wherein R¹ and R² taken together with N form a cyclic structure:
wherein B—D, D—E, E—G, G—J, two or more occurrences of J, and J—B are each independently joined by a single or double bond as valency and stability permit, wherein B is N, CH or C, D is —NR^{D}—, ═N—, —O—, —CHR^{D}—, or ═CR^{D}—, E is —NR^{E}—, ═N—, —O—, —CHR^{E}—, or ═CR^{E}—, G is —NR^{G}—, ═N—, —O—, —CHR^{G}—, or ═CR^{G}—, each occurrence of J is independently —NR^{J}—, ═N—, —O—, —CHR^{J}—, or ═CR—,
m is 0-4 and p is 0-4,
each occurrence of R³, R⁴, R^{D}, R^{E}, R^{G} and R^{J} is independently hydrogen, a protecting group, —(CR⁷R⁸)_{q}NR⁵R⁶, —(CR⁷R⁸)_{q}OR⁵, —(CR⁷R⁸)_{q}SR⁵, —(CR⁷R⁸)_{q}(C═O)R⁵, —(CR⁷R⁸)_{q}(C═O)OR⁵; —(CR⁷R⁸)_{q}(C═O)NR⁵R⁶, —(CR⁷R⁸)_{q}; —S(O)₂R⁵(CR⁷R⁸)_{q}NR⁵(C═O)R⁶, —(CR⁷R⁸)_{q}NR⁵(C═O)OR⁶, —(CR⁷R⁸)_{q}S(O)₂NR⁵R⁶, —(CR⁷R⁸)_{q}NR⁵S(O)₂R⁶, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
q is 0-4; and
each occurrence of R⁵, R⁶, R⁷ and R⁸ is independently hydrogen, a protecting group, or an aliphatic, heteroalipahtic, aryl, heteroaryl, -(aliphatic)aryl, -(aliphatic)heteroaryl, -(heteroalipahtic)aryl, or -(heteroaliphatic)heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

In yet other embodiments, compounds and libraries of special interest include those compounds and libraries as generally described above, in which L is 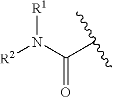
and one or both of R¹ and R² is a moiety having one of the following structures, or wherein R¹ and R² taken together with N form a cyclic moiety having one of the following structures:

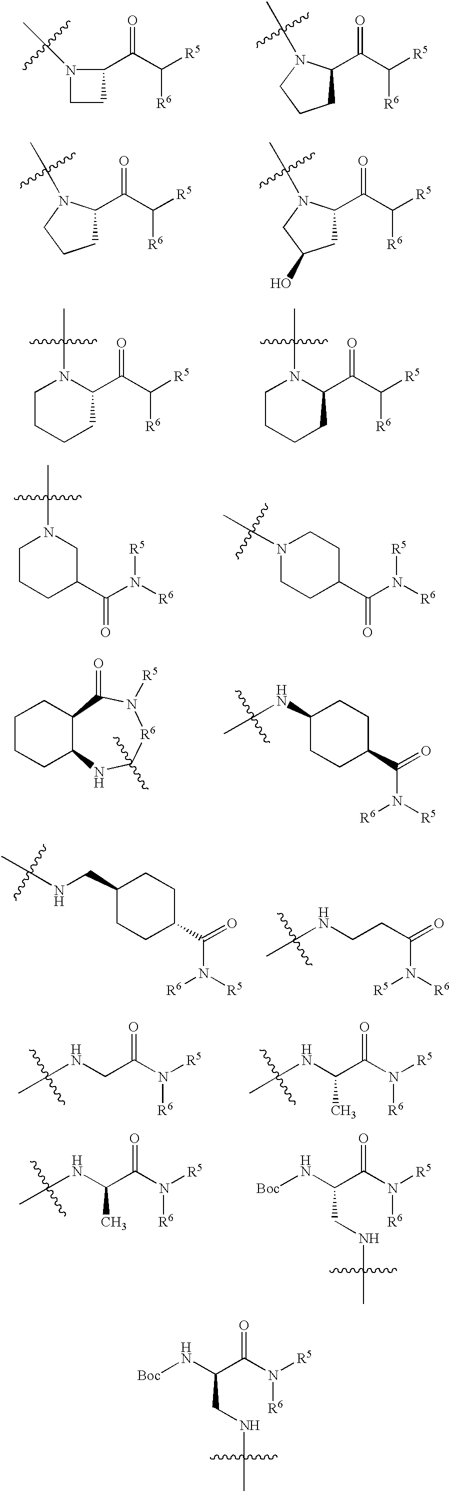

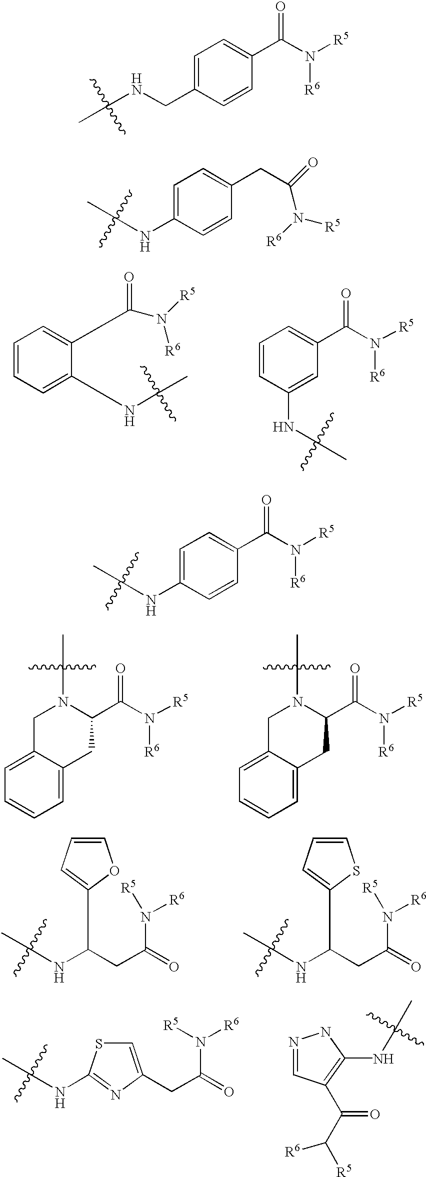

In still other embodiments, compounds and libraries of special interest include those compounds and libraries as generally described above, in which L is 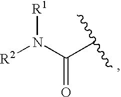
and R¹ and R² are each independently hydrogen or a cycloaliphatic, heterocycloaliphatic, aryl or heteroaryl moiety optionally substituted with a substituted heteroaryl moiety.

In still other embodiments, compounds and libraries of special interest include those compounds and libraries as generally described above, in which the substituted heteroaryl moiety has one of the structures: 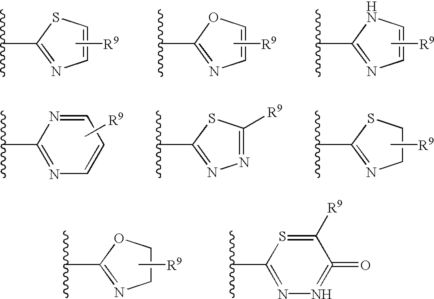
wherein R⁹ is —COO(R¹⁰), —CO(R¹⁰), —CO(NR¹⁰OR¹¹), —NR¹⁰OR¹¹, —NR¹⁰COR¹¹, —OR¹⁰, or —SR¹⁰, wherein each occurrence of R¹⁰ is independently hydrogen, a protecting group, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

A number of important subclasses of each of the foregoing classes deserve separate mention; these subclasses include subclasses of the foregoing classes in which:
i) libraries of compounds as described directly above in which the library comprises at least 5 members;
ii) libraries of compounds as described generally above in which the library comprises at least 20 members;
iii) libraries of compounds as described generally above in which the library comprises at least 100 members;
iv) libraries of compounds as described generally above in which the library comprises at least 500 members;
v) libraries of compounds as described generally above in which the library comprises at least 1000 members;
vi) libraries of compounds as described generally above in which each library member has a different molecular weight;
vii) libraries of compounds as described generally above in which each library member has a mass that differs from another library member by at least 5 atomic mass units; and
viii) libraries of compounds as described generally above in which each library member has a mass that differs from another library member by at least 10 atomic mass units;
ix), compounds and libraries of compounds, as described herein, in certain embodiments exclude compounds where L is 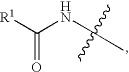
   and R¹ is any one of the following structures: 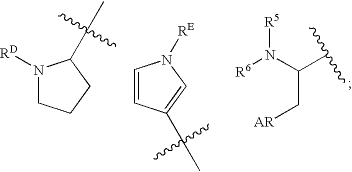
x) compounds and libraries of compounds, as described herein, in certain embodiments exclude compounds where L is 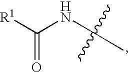
   and R¹ is any one of the following structures: 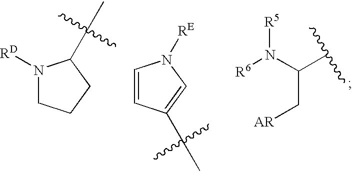
   and at least one of R^{D}, R^{E}, R⁵ or R⁶ is —SO₂-(alkyl) or —SO₂-(aryl).
compounds and libraries of compounds, as described herein, in certain embodiments exclude compounds having the structure: 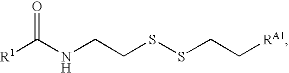
   where R^{A1} is NR^{A3}R^{A4} or NR^{A3}R^{A4}R^{A5}X⁻ wherein each occurrence of R^{A3}, R^{A4} and R^{A5} is hydrogen or a protecting group, and X is a halogen; and R¹ is one of the following:

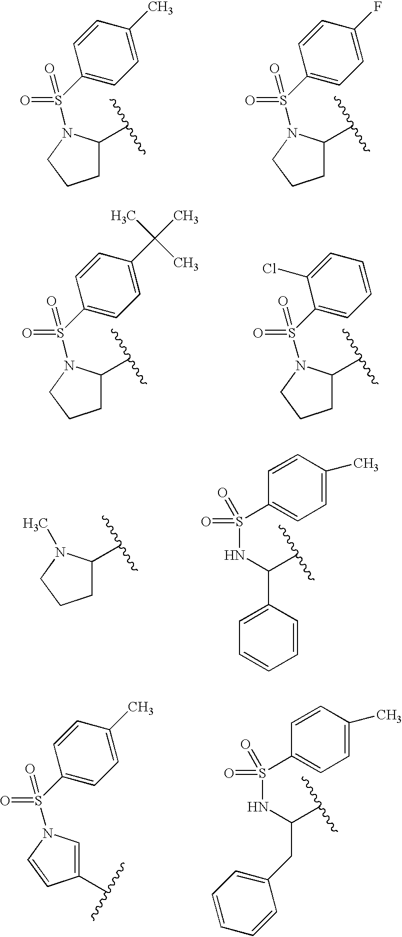

As the reader will appreciate, compounds of particular interest include, among others, those which share the attributes of one or more of the foregoing subclasses. Some of those subclasses are illustrated by the following sorts of compounds:

I) Compounds and Libraries of compounds of formula (I) described above in which :L is 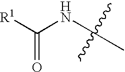
and R¹ has one of the following structures:

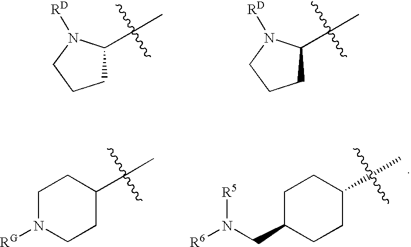

In certain embodiments of special interest, R^{D} and R^{G} are each independently hydrogen, a protecting group, —(CR⁷R⁸)_{q}S(O)₂R⁵, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, and wherein each occurrence of R⁵ and R⁶ is independently hydrogen, a protecting group or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

II) Compounds and Libraries of compounds of formula (I) described above in which L is 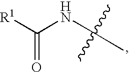
and R¹ has one of the following structures:

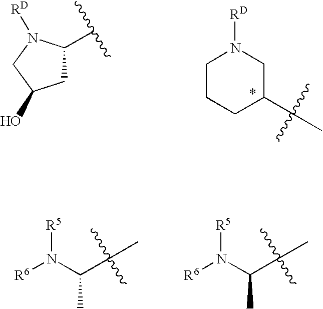

In certain embodiments of special interest, RD is hydrogen, a protecting group, —(CR⁷R⁸)_{q}S(O)₂R⁵; or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, and wherein each occurrence of R⁵ and R⁶ is independently hydrogen, a protecting group or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

III) Compounds and Libraries of compounds of formula (I) described above in which L is 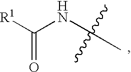
and R¹ has one of the following structures:

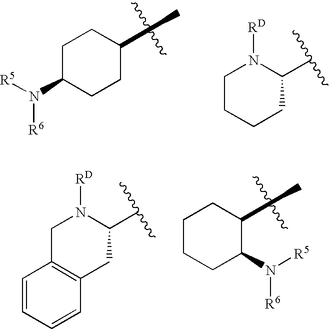

In certain embodiments of special interest, R^{D} is a protecting group, —(CR⁷R⁸)_{q}S(O)₂R⁵; or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, and wherein each occurrence of R⁵ and R⁶ is independently hydrogen, a protecting group or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

IV) Compounds and Libraries of compounds of formula (I) described above in which L is 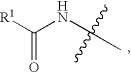
and R¹ has one of the following structures:

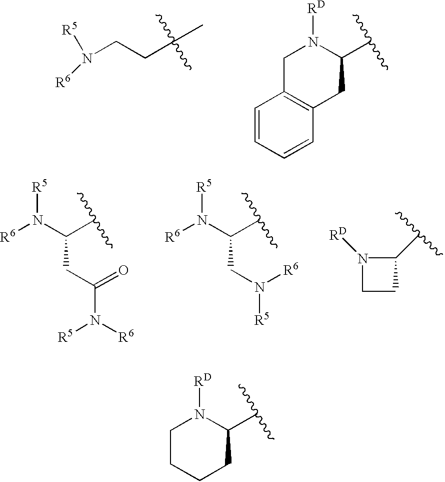

In certain embodiments of special interest, R^{D} is hydrogen, a protecting group, —(CR⁷R⁸)_{q}S(O)₂R⁵ or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, and wherein each occurrence of R⁵ and R⁶ is independently hydrogen, a protecting group or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

V) Compounds and Libraries of compounds of formula (I) described above in which L is 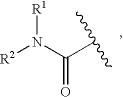
and R¹ has one of the following structures:

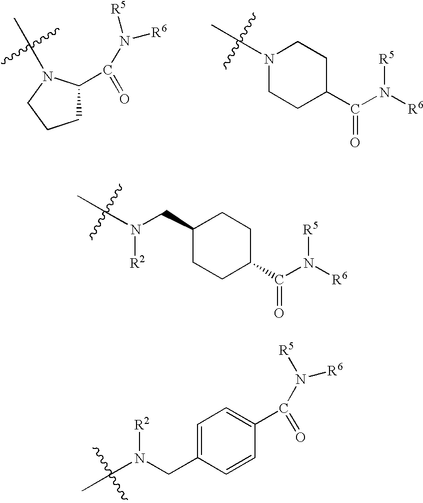

In certain embodiments of special interest, each occurrence of R², R⁵ and R⁶ is independently hydrogen, a protecting group or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

VI) Compounds and Libraries of compounds of formula (I) described above in which L is 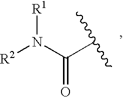
and R¹ is one of the following structures:

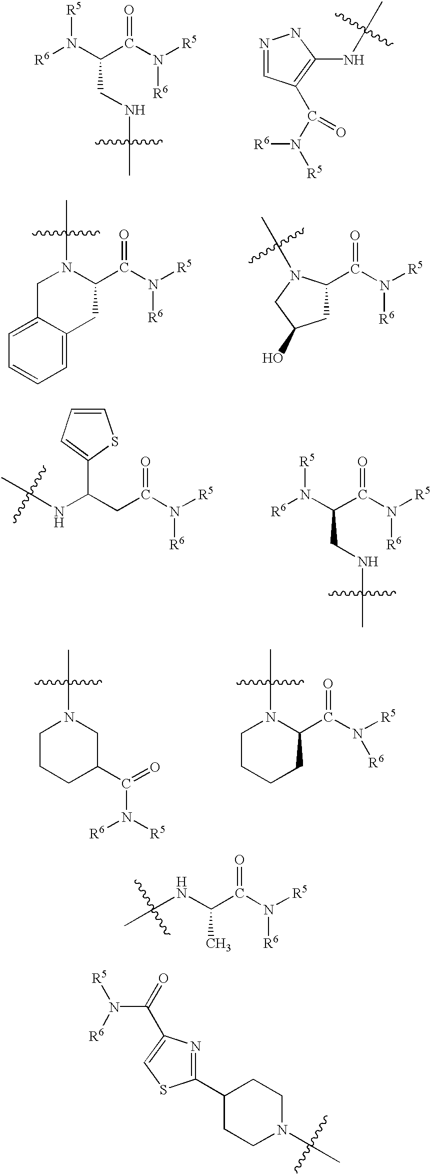

In certain embodiments of special interest, each occurrence of R⁵ and R⁶ is independently hydrogen, a protecting group or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

VI) Compounds and Libraries of compounds of formula (I) described above in which L is 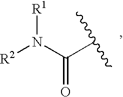
and R¹ is one of the following structures:

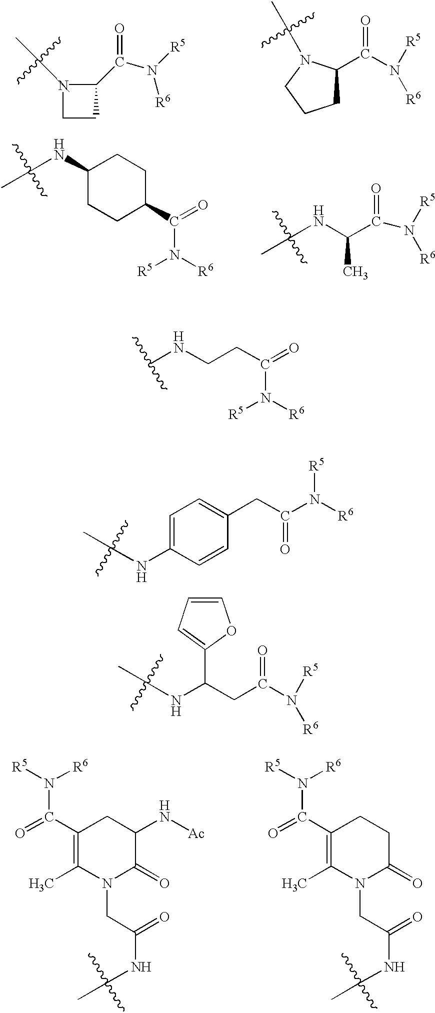

In certain embodiments of special interest, each occurrence of R⁵ and R⁶ is independently hydrogen, a protecting group or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

VII) Compounds and Libraries of compounds of formula (I) described above in which wherein R¹ and R² represent one of the following structures:

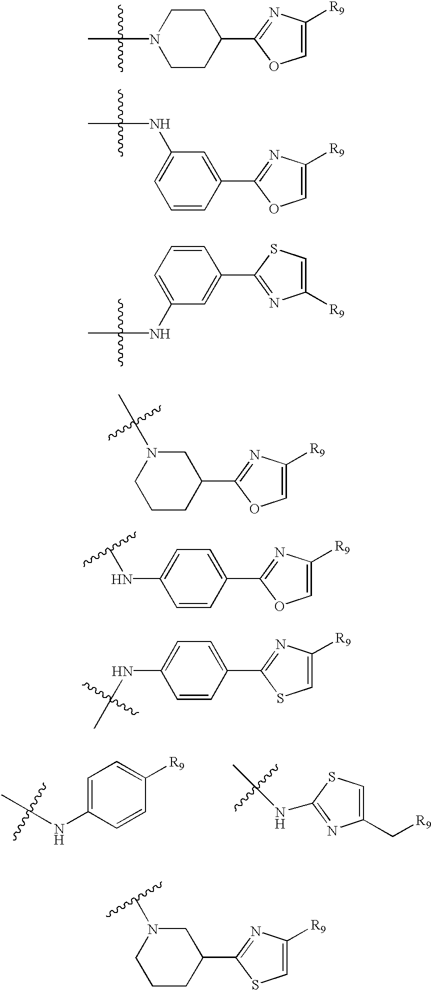

wherein R⁹ is COOH or is CO(NR¹⁰R¹¹), wherein each occurrence of R¹⁰ and R¹¹ is independently hydrogen, a protecting group, or an aliphatic, heteroaliphatic, aryl, heteroaryl, -(aliphatic)aryl, -(aliphatic)heteroaryl, -(heteroaliphatic)aryl, or -(heteroaliphatic)heteroaryl,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

It will also be appreciated that for each of the subgroups I-VII described above, a variety of other subclasses are of special interest, including, but not limited to those classes described above i)-xi) and classes, subclasses and species of compounds described above and in the examples herein.

Some of the foregoing compounds can exist in various isomeric forms, e.g., stereoisomers and/or diastereomers. Furthermore, certain compounds, as described herein may have one or more double bonds that can exist as either the Z or E isomer, unless otherwise indicated. The invention additionally encompasses the compounds as individual isomers (e.g., as either the R or S enantiomer) substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

2) Compounds and Definitions

As discussed above, this invention provides novel compounds and libraries of compounds useful in the drug discovery process. Compounds and libraries of this invention include those specifically set forth above and described herein, and are illustrated in part by the various classes, subgenera and species disclosed elsewhere herein.

It will be appreciated by one of ordinary skill in the art that asymmetric centers may exist in the compounds of the present invention. Thus, inventive compounds and pharmaceutical compositions thereof may be in the form of an individual enantiomer, diastereomer or geometric isomer, or may be in the form of a mixture of stereoisomers. Furthermore, it will be appreciated that certain of the compounds disclosed herein contain one or more double bonds and these double bonds can be either Z or E, unless otherwise indicated. In certain embodiments, the compounds of the invention are enantiopure compounds. In certain other embodiments, a mixture of stereoisomers or diastereomers are provided.

Additionally, the present invention provides pharmaceutically acceptable derivatives of the inventive compounds, and methods of treating a subject using these compounds, pharmaceutical compositions thereof, or either of these in combination with one or more additional therapeutic agents. The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety that is susceptible to removal in vivo yielding the parent molecule as the pharmacologically active species. An example of a pro-drug is an ester which is cleaved in vivo to yield a compound of interest. Pro-drugs of a variety of compounds, and materials and methods for derivatizing the parent compounds to create the pro-drugs, are known and may be adapted to the present invention.

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. The phrase “protected thiol” as used herein refers to a thiol that has been reacted with a group or molecule to form a covalent bond that renders it less reactive and which may be deprotected to regenerate a free thiol. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted”. whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of caspase-mediated disorders, as described generally above. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl-n, hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups 7:2 include, but are not limited to, ethynyl, 2-propynyl (propargyl), 1-propynyl and the like.

The term “alkoxy” (or “alkyloxy”), or “thioalkyl” as used herein refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. The term “dialkylamino” refers to a group having the structure —N(R′)₂, wherein R′ is alkyl, as defined herein. The term “aminoalkyl” refers to a group having the structure NH₂R′—, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R′; —CO₂(R′); —CON(R′)₂; —OC(O)R′; —OCO₂R′; —OCON(R′)₂; —N(R′)₂; —S(O)₂R′; —N(R′)S(O)₂R′, —S(O)₂R′N(R′)₂, —NR′(CO)R′ wherein each occurrence of R′ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl and the like. In certain embodiements of the present invention, the term “heteroaryl”, as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R′; —CO₂(R′); —CON(R′)₂; —OC(O)R′; —OCO₂R′; —OCON(R′)₂; —N(R′)₂; —S(O)₂R′; —N(R′)S(O)₂R′, —S(O)₂R′N(R′)₂, —NR′(CO)R′ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additionally, it will be appreciated, that any two adjacent groups taken together may represent a 4, 5, 6, or 7-membered cyclic, substituted or unsubstituted aliphatic or heteroaliphatic moiety. Additional examples of generally applicable substituents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or hetercyclic moieties, may optionally be substituted with substituents including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R′; —CO₂(R′); —CON(R′)₂; —OC(O)R′; —OCO₂R′; —OCON(R′)₂; —N(R′)₂; —S(O)₂R′; —N(R′)S(O)₂R′, —S(O)₂R′N(R′)₂, —NR′(CO)R′ wherein each occurrence of R′ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additionally, it will be appreciated that any of the cycloaliphatic or heterocycloaliphatic moieties described above and herein may comprise an aryl or heteroaryl moiety fused thereto. Additional examples of generally applicable substituents are illustrated by the specific embodiments shown in the Examples that are described herein.

The term “heteroaliphatic”, as used herein, refers to aliphatic moieties which contain one or more oxygen sulfur, nitrogen, phosphorus or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R′; —CO₂(R′); —CON(R′)₂; —OC(O)R′; —OCO₂R′; —OCON(R′)₂; —N(R′)₂; —S(O)₂R′; —N(R′)S(O)₂R′, —S(O)₂R′N(R′)₂, —NR′(CO)R′ wherein each occurrence of R′ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additionally, it will be appreciated that any of the cycloaliphatic or heterocycloaliphatic moieties described above and herein may comprise an aryl or heteroaryl moiety fused thereto. Additional examples of generally applicable substituents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6- or 7- membered ring or a polycyclic group, including, but not limited to a bi- or tr-cyclic group comprising fused six-membered rings having between one and three heteroatoms independently selected from oxygen, sulfur and nitrogen, wherein (i) each 5-membered ring has o to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a substituted or unsubstituted aryl or heteroaryl ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one or more of the hydrogen atoms thereon with but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R′; —CO₂(R′); —CON(R′)₂; —OC(O)R′; —OCO₂R′; —OCON(R′)₂; —N(R′)₂; —S(O)₂R; —N(R′)S(O)₂R′, —S(O)₂R′N(R′)₂, —NR′(CO)R′ wherein each occurrence of R′ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substitutents described above and herein may be substituted or unsubstituted. Additional examples or generally applicable substituents are illustrated by the specific embodiments shown in the Examples which are described herein.

The term “ligand candidate” refers to a compound that possesses or has been modified to possess a reactive group that is capable of forming a covalent bond with a complimentary or compatible reactive group on a target. The reactive group on either the ligand candidate or the target can be masked with, for example, a protecting group.

The phrase “site of interest” refers to any site on a target on which a ligand can bind. As used herein, a site of interest is any site that is outside of the primary binding site of a protein. For example, if a target is an enzyme, a site of interest is a site that is not the active site. If a target is a receptor, a site of interest is a site that is not the binding site of the receptor's ligand.

The terms “target,” “Target Molecule,” and “TM” are used interchangeably and in the broadest sense, and refer to a chemical or biological entity for which the binding of a ligand has an effect on the function of the target. The target can be a molecule, a portion of a molecule, or an aggregate of molecules. The binding of a ligand may be reversible or irreversible. Specific examples of target molecules include polypeptides or proteins (e.g., enzymes, including proteases, e.g. cysteine, serine, and aspartyl proteases), receptors, transcription factors, ligands for receptors, growth factors, cytokines, immunoglobulins, nuclear proteins, signal transduction components (e.g., kinases, phosphatases), allosteric enzyme regulators, and the like, polynucleotides, peptides, carbohydrates, glycoproteins, glycolipids, and other macromolecules, such as nucleic acid-protein complexes, chromatin or ribosomes, lipid bilayer-containing structures, such as membranes, or structures derived from membranes, such as vesicles. The definition specifically includes Target Biological Molecules (“TBMs”) as defined below.

A “Target Biological Molecule” or “TBM” as used herein refers to a single biological molecule or a plurality of biological molecules capable of forming a biologically relevant complex with one another for which a small molecule agonist or antagonist has an effect on the fuction of the TBM. In a preferred embodiment, the TBM is a protein or a portion thereof or that comprises two or more amino acids, and which possesses or is capable of being modified to possess a reactive group that is capable of forming a covalent bond with a compound having a complementary reactive group. Illustrative examples of TBMs include: enzymes, receptors, transcription factors, ligands for receptors, growth factors, immunoglobulins, nuclear proteins, signal transduction components, glycoproteins, glycolipids, and other macromolecules, such as nucleic acid-protein complexes, chromatin or ribosomes, lipid bilayer-containing structures, such as membranes, or structures derived from membranes, such as vesicles. The target can be obtained in a variety of ways, including isolation and purification from natural source, chemical synthesis, recombinant production and any combination of these and similar methods.

Preferred protein targets include: cell surface and soluble receptor proteins, such as lymphocyte cell surface receptors; enzymes; proteases (e.g., aspartyl, cysteine, metallo, and serine); steroid receptors; nuclear proteins; allosteric enzymes; clotting factors; kinases (serine/threonine kinases and tyrosine kinases); phosphatases (serine/threonine, tyrosine, and dual specificity phosphatases, especially PTP-1B, TC-PTP and LAR); thymidylate synthase; bacterial enzymes, fungal enzymes and viral enzymes (especially those associated with HIV, influenza, rhinovirus and RSV); signal transduction molecules; transcription factors; proteins or enzymes associated with DNA and/or RNA synthesis or degradation; immunoglobulins; hormones; and receptors for various cytokines. Illustrative examples of receptors include for example, erythropoietin (EPO), granulocyte colony stimulating (G-CSF) receptor, granulocyte macrophage colony stimulating (GM-CSF) receptor, thrombopoietin (TPO), interleukins, e.g. IL-2, IL-3, IL-4, IL-5, IL-6, IL-10, IL-11, IL-12, growth hormone, prolactin, human placental lactogen (LPL), CNTF, oncostatin, RANTES, MIPb, IL-8, insulin, insulin-like growth factor 1 (IGF-1), epidermal growth factor (EGF), heregulin-a and heregulin-b, vascular endothelial growth factor (VEGF), placental growth factor (PLGF), tissue growth factors (TGF-a and TGF-β), and nerve growth factor (NGF). Other targets include various neurotrophins and their ligands, other hormones and receptors such as, bone morphogenic factors, follicle stimulating hormone (FSH), and luteinizing hormone (LH), CD40 ligand, apoptosis factor-i and -2 (AP-1 and AP-2), p53, bax/bc12, mdm2, caspases (1, 3, 8 and 9), cathepsins, IL-1/IL-1 receptor, BACE, HIV integrase, PDE IV, Hepatitis C helicase, Hepatitis C protease, rhinovirus protease, tryptase, cPLA (cytosolic Phospholipase A2), CDK4, c-jun kinase, adaptors such as Grb2, GSK-3, AKT, MEKK-1, PAK-1, raf, TRAF's 1-6, Tie2, ErbB 1 and 2, FGF, PDGF, PARP, CD2, C5a receptor, CD4, CD26, CD3, TGF-alpha, NF-kB, IKK beta, STAT 6, Neurokinnin-1, CD45, Cdc25A, SHIP-2, human p53, bax/bc12, IgE/IgER, ZAP-70, ick, syk, ITK/BTK, TACE, Cathepsin S, K and F, CD11a, LFA/ICAM, VLA-4, CD28/B7, CTLA4, TNF alpha and beta, (and the p55 and p75 TNF receptors), CD40L, p38 map kinase, IL-2, IL-4, 11-13, IL-15, Rac 2, PKC theta, IL-8, TAK-1, jnk, IKK2 and IL-18.

3) Synthesis of Inventive Compounds and Libraries of Compounds:

As described in more detail in the Exemplification herein, a variety of tethering reagents and libraries of reagents (which compounds and libraries are described in detail above) can be prepared. In general, these tethering reagents and libraries of reagents are prepared by derivatizing desired building blocks with a suitable linker. It will be appreciated that a variety of building blocks can be utilized for the tethering reagents and libraries of reagents. For example, alkyl acids, aryl acids, primary alkyl amines, secondary alkyl amines, secondary aryl amines, aldehydes and ketones can be utilized as described in more detail above and herein. It will be appreciated that each of these building blocks may be purchased from a commercial source, or may be synthesized to generate a building block of particular interest. In addition, building blocks that are purchased from a commercial source may also be further derivatized to generate additional diverstiy (see “1+nub” chemistry, and the synthesis of “N-side” and “C-side” compounds and libraries as described in the exemplification herein).

Certain exemplary linkers for use in the invention (the synthesis of which are described in the exemplification herein) include, but are not limited to the following linkers shown directly below:

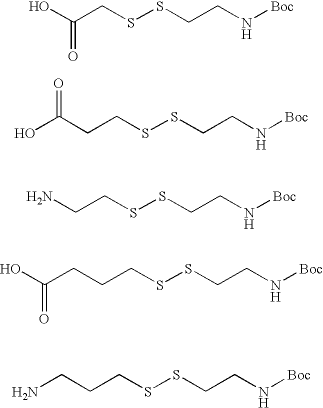

It will be appreciated that the amine linkers are generally employed for building blocks bearing a carboxylate, sulfonylchloride or isocyanate, while carboxylate linkers are generally employed for the derivatization of amines. It will also be appreciated that the length of the linker can be varied as necessary to sample the surface of a given protein, or more generally, of a target of interest. In general, standard coupling conditions are utilized to couple a desired building block and a desired linker as described in more detail herein. It will also be appreciated that once desired building blocks are attached to appropriate linkers, these building blocks can be further derivatized to “customize” reagents, as described in more detail herein.

3) Uses

As described above, the present invention provides novel compounds and libraries of compounds that are useful in the development of novel drug leads using the tethering method.

The general tethering method relies upon the formation of a covalent bond between the target and a potential ligand. The covalent bond that is formed between the target and the potential ligand allows the facile determination of both binding stoichiometry and binding location. The tethering method is described in U.S. Pat. No. 6,335,155, PCT Publication No. WO 00/00823, and Erlanson et al., Proc. Nat. Acad. Sci. USA 97:9367-9372 (2000) which are all incorporated herein by reference and is described briefly below. In general, the compounds and libraries of compounds are useful in the above-described method. Thus, in another embodiment of the invention, a method for ligand discovery is provided comprising: 1) contacting a target that comprises a chemically reactive group at or near a site of interest with a compound or library of compounds as described herein, which compound or library of compounds is capable of forming a covalent bond with a chemically reactive group; 2) forming a covalent bond between the target and the compound thereby forming a target-compound conjugate; and 3) identifying the target compound conjugate.

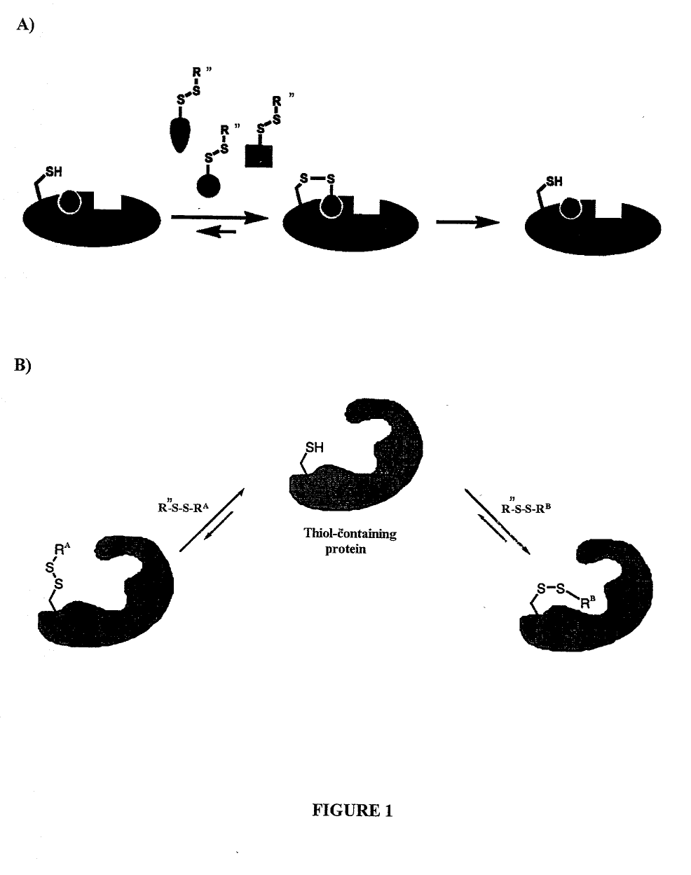

FIG. 1 schematically illustrates one embodiment of the tethering method. In this case, the target is a protein and the covalent bond is a disulfide bond. As shown, a thiol-containing protein is reacted with a plurality of ligand candidates. Ligand candidates are potential ligands that have been modified to include a moiety that is capable of forming a disulfide bond. This moiety can be a thiol group or a masked thiol such as a disulfide of the formula —SSR″ where R″ is unsubstituted C₁-C₁₀ aliphatic, substituted C₁-C₁₀ aliphatic, unsubstituted aryl or substituted aryl. In certain embodiments, R″ is selected to enhance the solubility of the potential ligand candidates. Illustrative examples of ligand candidates include those as described in detail above and herein. In certain exemplary embodiments, ligand candidates include, but are not limited to: 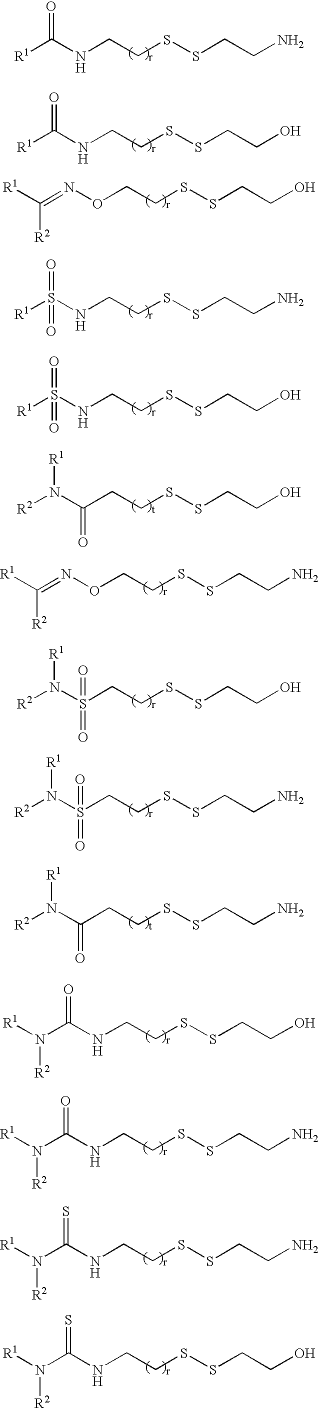
wherein r is 1 or 2; and t is 0, 1 or 2.

It will also be appreciated that once a ligand candidate is identified using the tethering method described above, tethered compounds as described above may be characterized using X-ray crystallography methods. When using X-ray crystallography as a characterization method (or other characterization methods), it is desirable to obtain homogeneous compounds after exposure to reducing conditions. Thus, in certain embodiments, compounds and libraries of special interest include those compounds and libraries wherein 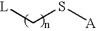
represents one of the structures having a substituted thiolate moiety, which moiety, upon exposure to reducing conditions, results in homogeneous compounds: 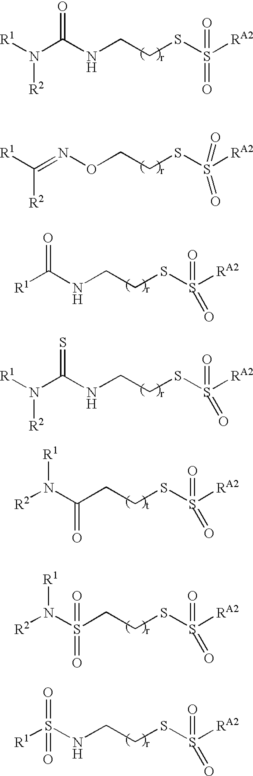
wherein r is 1 or 2; and R^{A2} is an alkyl, heteroalkyl, aryl, herteroaryl, -(alkyl)aryl, -(alkyl)heteroaryl, -(heteroalkyl)aryl, or -(heteroalkyl)heteroaryl moiety.

In certain embodiments of special interest R^{A2} is methyl or phenyl.

As shown, a ligand candidate that possesses an inherent binding affinity for the target is identified and a corresponding ligand that does not include the disulfide moiety is made comprising the identified binding determinant (represented by the circle).

FIG. 1B schematically illustrates the theory behind tethering. When a thiol-containing protein is equilibrated with at least one disulfide-containing ligand candidate, equilibrium is established between the modified and unmodified protein. In preferred embodiments, the reaction occurs in the presence of a reducing agent. If the ligand candidate does not have an inherent binding affinity for the target protein, the equilibrium is shifted toward the unmodified protein. In contrast, if the ligand candidate does have an inherent affinity for the protein, equilibrium shifts toward the modified protein. Both situations are illustrated in FIG. 1B. In the first, the R^{A} moiety of the ligand candidate possesses little or no binding affinity for the protein. Thus, the formation of the protein-ligand conjugate is a function of the probability of forming a disulfide bond given the concentration of the protein, the ligand candidate, and reducing agent. In the second, the R^{B} moiety of the ligand candidate possesses an inherent binding affinity for the protein. Consequently, once the disulfide bond is formed between the protein and the ligand candidate, the protein-ligand conjugate is stabilized. Thus, equilibrium is shifted toward the formation of the protein-ligand conjugate.

To further illustrate tethering, the method has been applied to thymidylate synthase (“TS”), an essential enzyme for virtually all living organisms. TS, along with dihydrofolate reductase (“DHFR”) and serine hydroxymethylase, forms a biochemical functional unit, the thymidylate synthase cycle, that provides the sole de novo pathway for synthesis of the DNA base thymidine 5′-monophosphate (“dTMP”) from the RNA base dUMP. Both TS and DHRF are targets for anticancer drug development. Because the TS gene is also found in many viruses, it is also a target for development of anti-parasitic, anti-fungal, and anti-viral agents.

TS is an ideal validating target for several reasons. First, numerous high resolution crystal structures of various TS enzymes have been determined so that structural information can be incorporated into compound design. Second, a simple colorimetric assay exists for determining whether a potential ligand binds to TS. This assay depends on the rate of conversion of 5,10-CH₂—H₄folate to H₂folate in the presence of dUMP. A second assay for binding is also spectrophotometric and relies on competition with pyridoxal-5′-phosphate (“PLP”), which forms a complex with TS with a unique spectral signature.

The TS chosen for the purposes of illustration is the E. coli TS. Like all TS enzymes, it contains a naturally occurring cysteine residue in the active site (Cys146) that can be used for tethering. The E. coli TS includes four other cysteines but these are not conserved among other TS enzymes and are buried and thus not accessible. However, if one or more of these cysteines were reactive toward disulfides, then mutant versions of these enzymes can be used where these cysteines are mutated to another amino acid such as alanine.

In the first experiment, wildtype TS and the C146S mutant (wherein the cysteine at position 146 has been mutated to serine) were contacted with cystamine, H₂NCH₂CH₂SSCH₂CH₂NH₂. The wildtype TS enzyme reacted cleanly with one equivalent of cystamine while the mutant TS did not react indicating that the cystamine was reacting with and was selective for Cys-146.

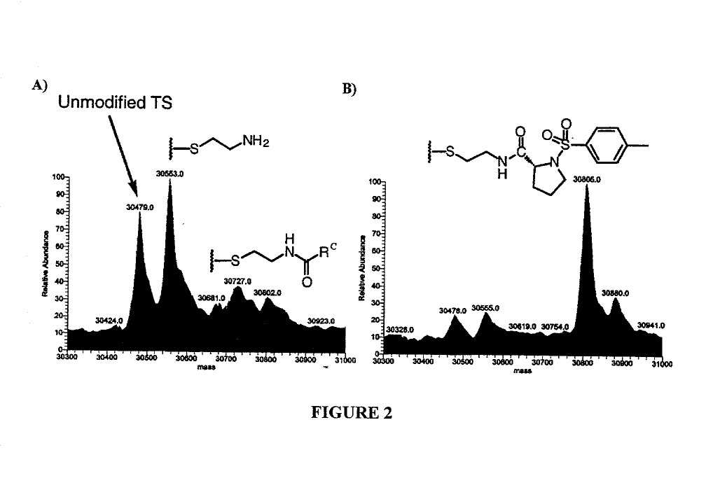

The wildtype TS was subjected to several tethering experiments with different pools of ligand candidates. FIG. 2 illustrates two representative tethering experiments wherein the ligand candidates were of the formula

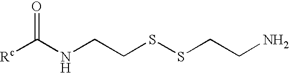

This is a specific embodiment of the genus of ligand candidates of the formula RSSR where R corresponds to R^{c}C(═O)NHCH₂CH₂— and R″ corresponds to —CH₂CH₂NH₂. R^{c} is unsubstituted C₁-C₁₀ alkyl, substituted C₁-C₁₀ alkyl, unsubstituted aryl, or substituted aryl, and is the variable moiety among this pool of library members.

FIG. 2A is the deconvoluted mass spectrum of the reaction of TS with a pool of 10 different ligand candidates with little or no binding affinity for TS. In the absence of any binding interactions, the equilibrium in the disulfide exchange reaction between TS and an individual ligand candidate is to the unmodified enzyme. This is schematically illustrated by the following equation.

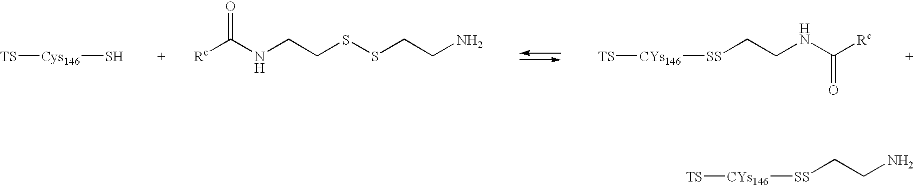

As expected, the peak that corresponds to the unmodified enzyme is one of two most prominent peaks in the spectrum. The other prominent peak is TS where the thiol of Cys146 has been modified with cysteamine. Although this species is not formed to a significant extent for any individual library member, the peak is due to the cumulative effect of the equilibrium reactions for each member of the library pool. When the reaction is run in the presence of a thiol-containing reducing agent such as 2-mercaptoethanol, the active site cysteine can also be modified with the reducing agent. Because cysteamine and 2-mercaptoethanol have similar molecular weights, their respective disulfide bonded TS enzymes are not distinguishable under the conditions used in this experiment. The small peaks on the right correspond to discreet library members. Notably, none of these peaks are very prominent. FIG. 2A is characteristic of a spectrum where none of the ligand candidates possesses an inherent binding affinity for the target.

FIG. 2B is the deconvoluted mass spectrum of the reaction of TS with a pool of 10 different ligand candidates where one of the ligand candidates possesses an inherent binding affinity to the enzyme. As can be seen, the most prominent peak is the one that corresponds to TS where the thiol of Cys146 has been modified with the N-tosyl-D-proline compound. This peak dwarfs all others including those corresponding to the unmodified enzyme and TS where the thiol of Cys146 has been modified with cysteamine. FIG. 2B is an example of a mass spectrum where tethering has captured a moiety that possesses a strong inherent binding affinity for the desired site.

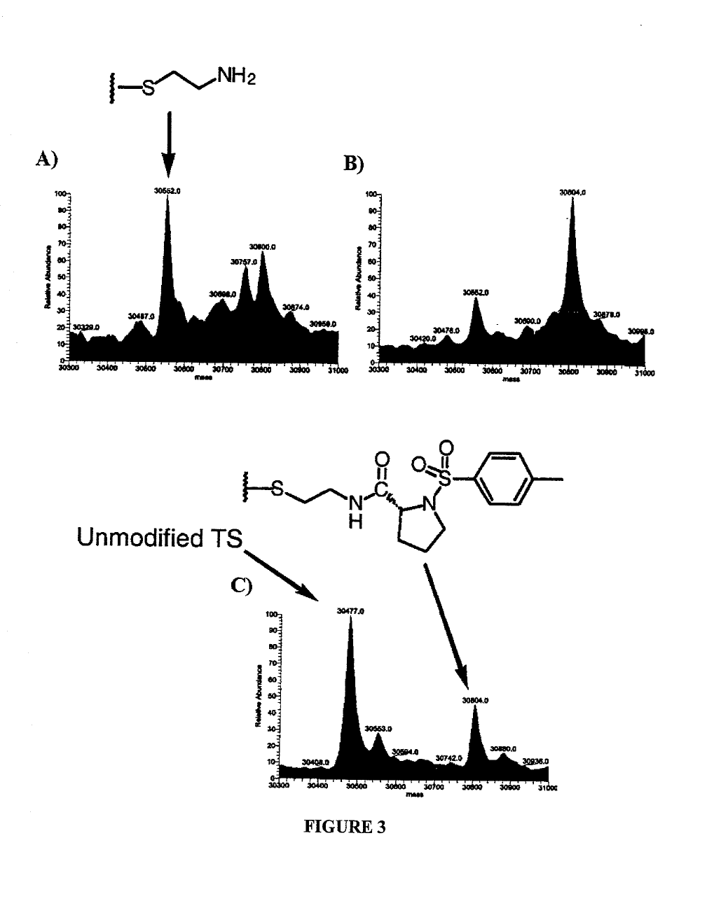

When tethering occurs in the presence of a reducing agent, the process becomes more thermodynamically driven and equilibrium-controlled. FIG. 3 is an illustration of this phenomenon and shows three experiments where TS is reacted with the same library pool containing the selected N-tosyl-D-proline compound in the presence of increasing concentration of the reducing agent, 2-mercaptoethanol.

FIG. 3A is the deconvoluted mass spectrum when the reaction is performed without 2-mercaptoethanol. The most prominent peak corresponds to TS that has been modified with cysteamine. However, the peak corresponding to N-tosyl-D-proline is nevertheless moderately selected over the other ligand candidates. FIG. 3B is the deconvoluted mass spectrum when the reaction is in the presence of 0.2 mM 2-mercaptoethanol. In contrast, to the spectrum in FIG. 3A, the peak corresponding to N-tosyl-D-proline is the most prominent peak and thus is strongly selected over the other ligand candidates. Finally, FIG. 3C is the deconvoluted mass spectrum when the reaction is in the presence of 20 mM 2-mercaptoethanol. Not surprisingly, the most prominent peak under such strongly reducing conditions is the unmodified enzyme. Nevertheless, the peak corresponding to N-tosyl-D-proline is still selected over that of the other ligand candidates in the library pool.

FIG. 3 highlights the fact that the degree of cysteine modification in a target protein by a particular ligand candidate that possesses an inherent affinity for the target is, in part, a function of the reducing agent concentration. In general, the higher the binding affinity of the ligand candidate for the target protein, the higher the concentration of reducing agent that can be used and still get strong selection. As a result, the concentration of the reducing agent used in the tethering screen can be used as a surrogate for binding affinity as well as to set a lower limit of binding affinity the ligand candidate must have to be strongly selected.

As stated previously, the tethering method can be used with a single ligand candidate or a plurality of ligand candidates. In preferred embodiments, the tethering method is used to screen a plurality of ligand candidates (e.g., 5, 20, 100, 500, 1000, and even >1000) to maximize throughput and efficiency.

A structure-activity relationship (“SAR”) can be developed using information from a tethering experiment in much the same way SAR is developed using traditional assays. For example, ligand candidates with R^{c}s on the left hand side of the scheme below were strongly selected against the E. coli TS but those ligand candidates with R^{c}s on the right hand side were not.

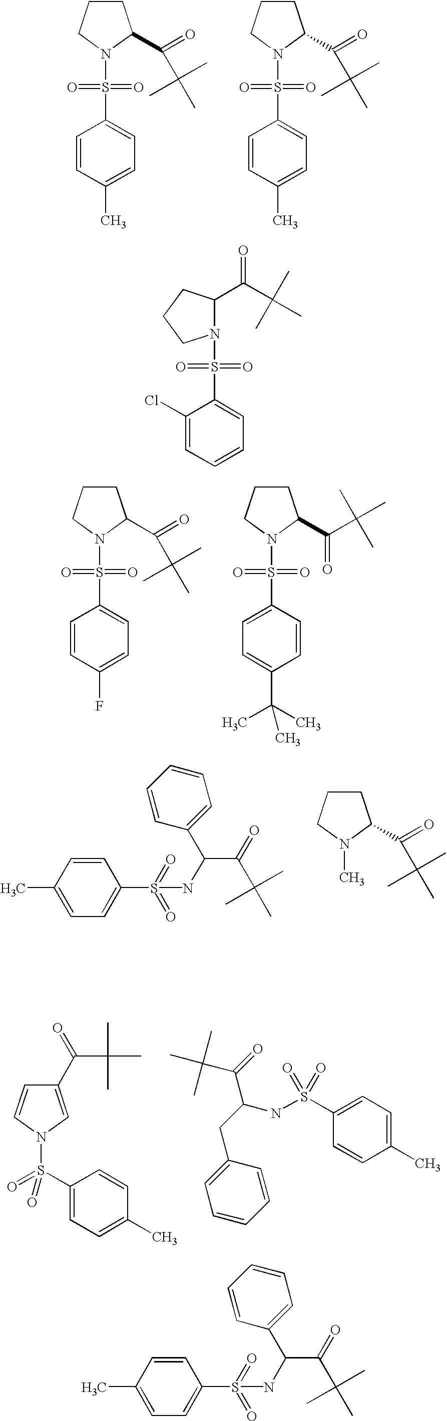

Based on the data from screening approximately 1200 compounds, it was determined that the phenyl-sulfonamide core and the proline ring are essential. For example, although TS appears to accommodate a great deal of flexibility around the phenyl ring where the phenyl ring can be unsubstituted or substituted with a range of groups including methyl, t-butyl, and halogen, its presence is required for selection. Similarly, the proline ring appears essential because compounds where it was replaced with phenylalanine, phenylglycine or pyrrole were not selected.

In addition to the above, further experiments were performed to validate that the compounds selected from tethering correspond to those with binding affinity for the target. In one illustrative example, the tethering experiment is performed in the presence of a known substrate. If the selected ligand candidate possesses an inherent binding affinity for the target, it would be resistant to displacement by the substrate. In contrast, a ligand candidate that lacks an inherent binding affinity or cysteamine would be easily displaced by the substrate. Another illustrative example is traditional enzymatic assays on the tether-free analog. For example, the affinity of the R^{c} portion of the ligand fragment was determined using Michaelis-Mention kinetics. The Kᵢ of the free acid 1 was 1.1±0.25 mM. Notably, the free acid competed with the natural substrate dUMP. Thus, N-tosyl-D-proline 1 is a weak but competitive inhibitor of TS

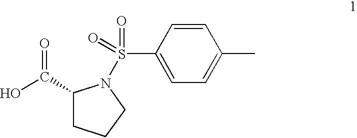

In another embodiment, the naturally occurring cysteine residue in the active site was mutated to a serine (C146S) and another cysteine was introduced (L143C or H147C). Tethering using the C146S/L143C mutant produced similar results as the wild type enzyme. Notably, the N-tosyl-D-proline analog was strongly selected. In contrast, the C146S/H147C did not select the N-tosyl-D-proline analog but several other molecules were selected. These results are believed to reflect the differences in the local binding environment surrounding the reactive cysteine and the geometric constraints of the disulfide linker.

X-ray crystallography was used to solve the three-dimensional structures of the native enzyme and several complexes to confirm that the information obtained from tethering can be correlated with productive binding to the target. Table 1 details crystallographic data and refinement parameters. One complex was of the free acid of N-tosyl-D-proline bound to TS (fourth entry in Table 1). Another complex was of the N-tosyl-D-proline derivative tethered to the active site cysteine (Cys-146) (second entry in Table 1). Yet another complex was of N-tosyl-D-proline derivative tethered to the C146S/L143C mutant (third entry in Table 1).

TABLE 1

| | | | | | | | | | | | rms | rms |
|---|---|---|---|---|---|---|---|---|---|---|---|---|
| | | | | | | | | | | | devi- | devi- |
| | | | | | | | | | | | ation | ation |
| | | Cell | Resolu- | Reflections | | Complete- | | | | | bond | bond |
| | Space | dimensions, | tion, | (no.) | | ness,^{†} | R_{sym} | | R_{cryst},^{§} | R_{free},^{¶} | lengths, | angles, |
| Data set | group* | Å | Å | Overall | Unique | % | (ƒ), ^{‡} % | l/σ | % | % | Å | deg |
| Native | 12₁3 | a = 131.17 | 10 − 1.75 | 104,019 | 36,586 | 96.7 (91.6) | 4.9 (33.8) | 20.5 (4.0) | 19.8 | 24.4 | 0.010 | 2.30 |
| C146 | P6₃ | a = 126.22 c = 67.02 | 10 − 2.00 | 97,445 | 41,001 | 98.8 (94.5) | 4.4 (26.0) | 14.7 (4.1) | 19.8 | 26.8 | 0.010 | 2.59 |
| tethered | | | | | | | | | | | | |
| N-tosyl-o- | | | | | | | | | | | | |
| proline | | | | | | | | | | | | |
| L143C | P6₃ | a = 126.33 c = 67.12 | 10 − 2.15 | 78,793 | 32,045 | 96.7 (92.1) | 8.1 (28.6) | 12.8 (4.5) | 19.6 | 26.7 | 0.014 | 3.06 |
| tethered | | | | | | | | | | | | |
| N-tosyl-o- | | | | | | | | | | | | |
| proline | | | | | | | | | | | | |
| Non- | 12₁3 | a = 131.88 | 10 − 1.90 | 202,300 | 31,422 | 100 (100) | 7.4 (28.2) | 19.7 (3.8) | 19.2 | 23.8 | 0.011 | 2.49 |
| covalent | | | | | | | | | | | | |
| N-tosyl-o- | | | | | | | | | | | | |
| proline | | | | | | | | | | | | |
| Glu-TP | P6₃ | a = 126.14 c = 66.81 | 10 − 2.00 | 143,599 | 40,497 | 99.4 (96.9) | 8.5 (31.9) | 13.9 (4.0) | 19.4 | 25.1 | 0.007 | 2.15 |
| Glu-TP-β- | P6₃ | a = 126.03 c = 66.84 | 10 − 1.75 | 142,016 | 58,487 | 95.8 (85.2) | 4.0 (22.5) | 17.1 (4.9) | 18.0 | 21.4 | 0.007 | 2.00 |
| Ala | | | | | | | | | | | | |

This is not a “true” free R factor because the starting model was a fully refined structure. However, the free R factor set of reflections was kept constant for each of the above refinements.
*The 12₁3 crystal contains one monomer per asymmetric unit. The P6₃ form contains the biologically relevant homodimer.
^{†} Values In parentheses are for the highest resolution bin.
^{‡}R_{sym} (ƒ) = Σₕₖₗ|/ₕₖₗ</_{hkl>}{/Σₕₖₗ/ₕₖₗ, where /ₕₖₗ is the intensity of reflection ₕₖₗ.
^{§}R_{cryst} = Σₕₖₗ||F_{obs}| - |F_{calc}||/|F_{obs}|, where F_{obs} and F_{calc} are the observed and calculated structure factors, respectively, for the data used in refinement.
^{¶}R_{free} = Σₕₖₗ||F_{obs}| - |F_{calc}||/|F_{obs}|, where F_{obs} and F_{calc} are the observed and calculated structure factors, respectively, for 10% of the data omitted from refinement.

Significantly, the location of the N-tosyl-D-proline moiety is very similar in all three cases (RMSD of 0.55-1.88 |, compared to 0.11-0.56 Å for all Cα carbons in the protein). The fact that the N-tosyl-D-proline substituents closely overlap while the alkyl-disulfide tethers converge onto this moiety from different cysteine residues supports the notion that the N-tosyl-D-proline moiety, not the tether, is the binding determinant.

As can be seen, tethering is a powerful method that can identify ligands that bind to a site of interest in a target. Tethering can be used alone or in combination with other medicinal chemistry methods to identify and optimize a drug candidate.

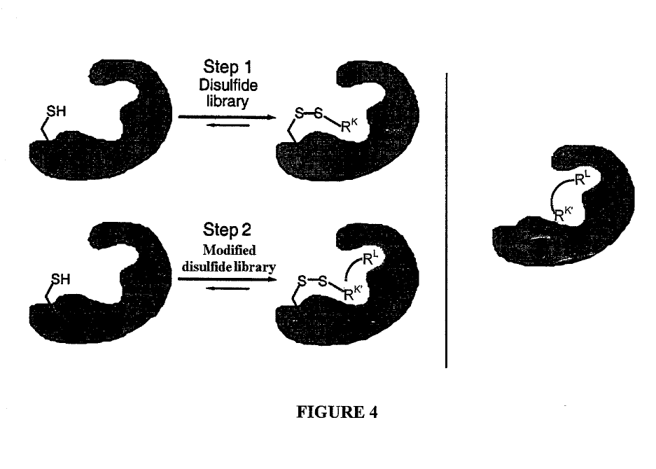

In one aspect of the present invention, tethering is used to identify a binding determinant (e.g. R^{c}) and then traditional medicinal chemistry is used to make higher affinity compounds containing the identified binding determinants or variations thereof. In one embodiment, tethering is used to both identify a binding determinant and also used to assess whether compounds containing variations of the identified binding determinants bind to the target with higher affinity. In other words, tethering can be used as an alternative to traditional binding experiments where either functional assays are not available or are susceptible to artifacts. This approach is schematically illustrated in FIG. 4. As can be seen, tethering is used to identify a binding determinant R^{D}. Once such a binding determinant is identified, traditional medicinal chemistry approaches are used to synthesize variants of R^{D} in a modified library. The modified library of ligand candidates would include variants of R^{D} such as isosteres and homologs thereof. The modified library can also include “extended” compounds that include R^{D} or variations thereof as well as other binding determinants that can take advantage of adjacent binding regions. FIG. 4 illustrates a selected compound from the modified library wherein the original binding determinant R^{K} was modified to R^{K′} and the selected compound includes a second binding determinant R^{L}.

An illustration of the approach outlined in FIG. 4 is as follows where derivatives of the selected N-tosyl-D-proline compound were made and tested as a series of ligand candidates using tethering. Based on the crystal structure of N-tosyl-D-proline bound to TS, the methyl group off the phenyl ring was in a promising location for use as a derivitization point. Eighty eight derivatives having six different linker lenths were synthesized and the inhibition constants of the untethered versions of the selected ligand candidates were determined. Two of the best compounds were:

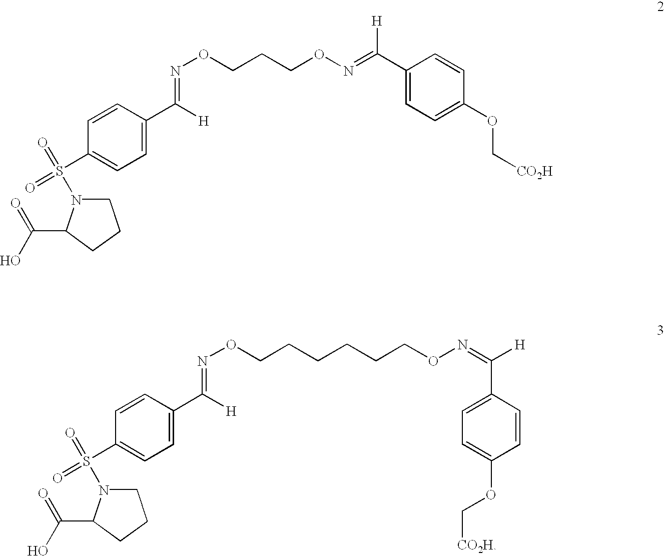

The Kᵢ of compound 2 was determined to be about 55 μM and the Kᵢ of compound 3 was determined to be about 40 μM.

In another aspect of the present invention, methods are provided for identifying two binding determinants that are subsequently linked together. In general, the method comprises:
a) identifying a first compound that binds to a target protein;
b) identifying a second compound that binds to the target protein; and,
c) linking the first compound and second compound through a linker element to form a conjugate molecule that binds to the target protein. In preferred embodiments, the conjugate molecule binds to the target protein with higher binding affinity than either the first compound or second compound alone.

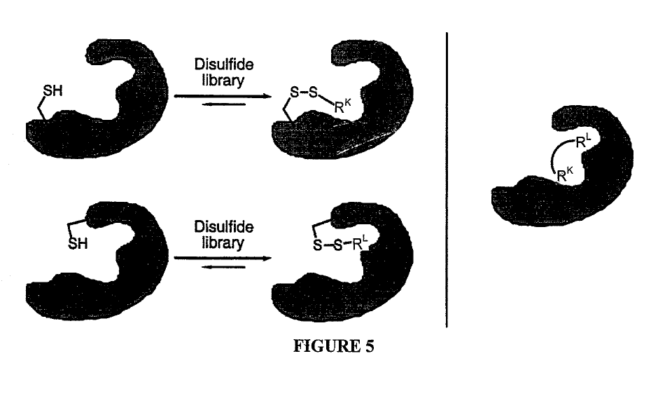

In one embodiment, the first compound is of the formula R^{K}SSR″ and the second compound is of the formula R^{L}SSR″ (where R″ is as previously described and R^{K} and R^{L} are each independently C₁-C₂₀ unsubstituted aliphatic, C₁-C₂₀ substituted aliphatic, unsubstituted aryl, or substituted aryl) and the first and second compounds bind to the target protein through a disulfide bond. FIG. 5 is a schematic illustration of this method where two separate tethering experiments are used to identify binding determinants R^{K} and R^{L} that are subsequently linked together to form a conjugate molecule that binds to the target protein. In another embodiment, the tethering experiments to identify binding determinants R^{K} and R^{L} occur simultaneously. In this way, it is assured that the two identified binding determinants bind to the target protein at non-overlapping sites.

In another embodiment, the first compound is identified using tethering and the second compound is identified through a non-tethering method. In one embodiment, the non-tethering method comprised rational drug design and traditional medicinal chemistry. The crystal structure of N-tosyl-D-proline bound to TS revealed that the tosyl group is in roughly the same position and orientation as the benzamide moiety of methylenetetrahydrofolate, the natural cofactor for the TS enzyme. Consequently, the glutamate moiety of methylenetetrahydrofoloate was grafted onto compound 1. Table 2 shows a selected number of these compounds.

TABLE 2

| COMPOUND | 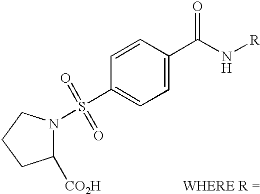 | Kᵢ |
|---|---|---|
| 4 (L-proline) | 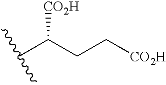 | 83 ± 5 μM |
| 5 (D-proline) | 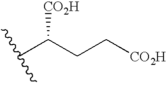 | 24 ± 7 μM |
| 6 | 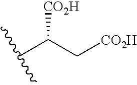 | 242 ± 3 μM |
| 7 | 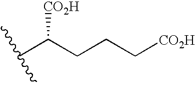 | 23 ± 6 μM |
| 8 | 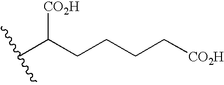 | 32 ± 2 μM |
| 9 | 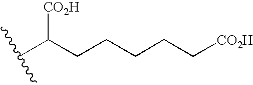 | 14 ± 6 μM |
| 10 | 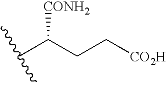 | 378 ± 69 μM |
| 11 | 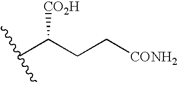 | 61 ± 14 μM |
| 12 | 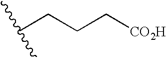 | 246 ± 46 μM |

There is a distinct preference for the D-enantiomer of proline (compound 5) over the L-enantiomer (compound 4) and the a-carboxylate of the glutamate residue is important because removing it (compound 12) or changing it to a primary amide (compound 10) correlates with a significant loss in binding affinity.

In another aspect of the present invention, a variation on the tethering method is provided for use in making and optimizing compoundsThe method comprises:
a) providing a target having a reactive nucleophile at or near a site of interest; and
b) contacting the target with an extender thereby forming a target-extender complex wherein the extender comprises a first functionality that reacts with the nucleophile in the target to form a covalent bond and a second functionality that is capable of forming a disulfide bond;
c) contacting the target-extender complex with a ligand candidate that is capable of forming a disulfide bond;
d) forming a disulfide bond between the target-extender complex and the ligand candidate thereby forming a target-extender-ligand conjugate; and
e) identifying the ligand candidate present in the target-extender-ligand conjugate. Optionally, the target is contacted with a ligand candidate in the presence of a reducing agent.

Illustrative examples of suitable reducing agents include but are not limited to: cysteine, cysteamine, dithiothreitol, dithioerythritol, glutathione, 2-mercaptoethanol, 3-mercaptoproprionic acid, a phosphine such as tris-(2-carboxyethyl-phosphine) (“TCEP”), or sodium borohydride. In one embodiment, the reducing agent is 2-mercaptoethanol. In another embodiment, the reducing agent is cysteamine. In another embodiment, the reducing agent is glutathione. In another embodiment, the reducing agent is cysteine.

In one embodiment, the target comprises a —SH as the reactive nucleophile and the extender comprises a first functionality that is capable of forming a covalent bond with the reactive nucleophile on the target and a second functionality that is capable of forming a disulfide bond. In another embodiment, the reactive nucleophile on the target is a naturally occurring —SH from a cysteine that is part of the naturally occurring protein sequence. In another embodiment, the reactive nucleophile on the target is an engineered -SH group where mutagenesis was used to mutate a naturally occurring amino acid to a cysteine.

In one embodiment, the first and second functionalities of the extender are each independently a —SH or a masked —SH. An illustrative example of a masked thiol is a disulfide of the formula —SSR″ where R″ is as previously described. In this embodiment, the covalent bond formed between the target and the extender is a disulfide bond and thus is a reversible covalent bond. In one variation of the method, the target is contacted with the extender prior to contacting the target-extender complex with one or more ligand candidates. In another variation, the target is contacted with a pool comprising the extender and one or more ligand candidates.

In another embodiment, the first functionality is a group that is capable of forming an irreversible covalent bond with the reactive nucleophile of the target under conditions that do not denature the target and the second functionality is a —SH or a masked —SH. In one embodiment, the first functionality is a group capable of undergoing S_{N}2-like addition. Illustrative example of such extenders include: (i) α-halo acids such as 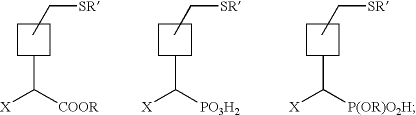
(ii) fluorophosphonates such as 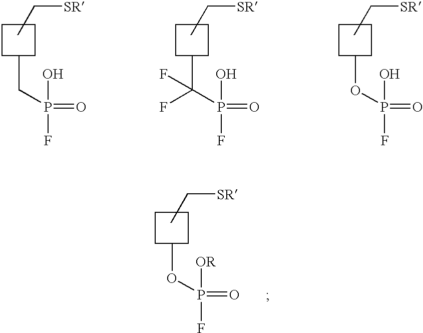
(iii) epoxides such as 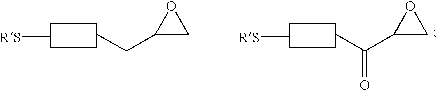
(iv) aziridines such as 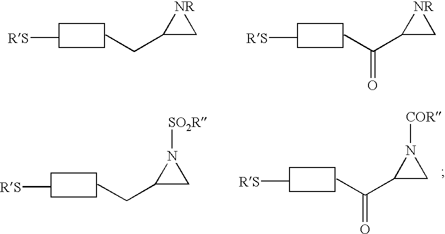
(v) thiiranes such as 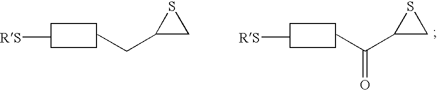
(vi) halomethyl ketones/amides such as 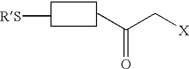
   where R is unsubstituted C₁-C₂₀ aliphatic, substituted C₁-C₂₀ aliphatic, unsubstituted aryl, and substituted aryl; R′ is H, —SR″ wherein R″ has been previously defined; and X is a leaving group. Illustrative examples of include halogen, N₂, OR, —P(═O)Ar₂, —NO(C═O)R, —(C═O)R, —SR and vinyl sulfones.
   In another embodiment, the first functionality is a group capable of undergoing SN aryl like addition. Illustrative examples of suitable groups include 7-halo-2,1,3-benzoxadiazaoles, and ortho/para nitro substituted halobenzenes such as 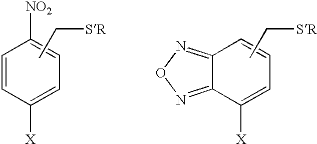
   where R′ and X are as previously defined.

In another embodiment, the first functionality is a group capable of undergoing Michael-type addition. Illustrative examples of suitable groups include any moiety that includes a double or triple bond adjacent to an electron withdrawing system such as a carbonyl, imines, quinines, CN, NO₂, and —S(═O)—. Illustrative examples of such extenders include: 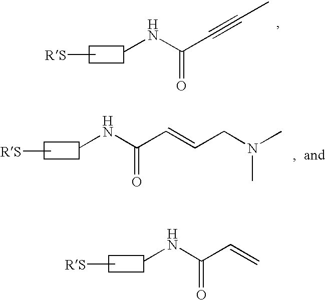
where R′ is as previously defined.

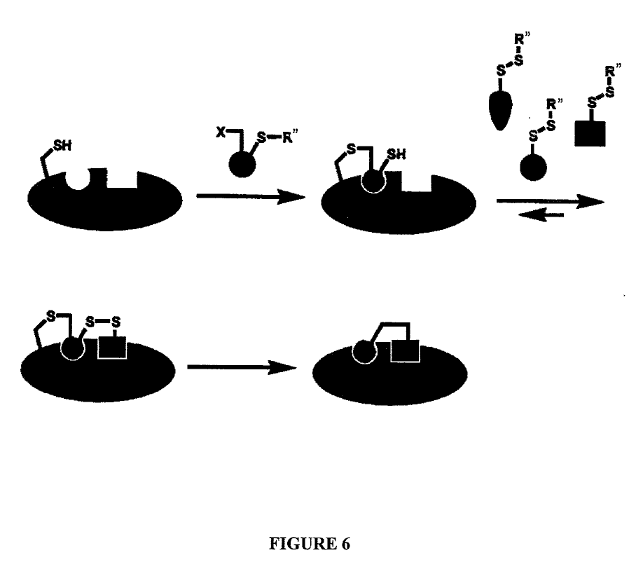

FIG. 6 illustrates one embodiment of the tethering method using extenders. As shown, a target that includes a reactive nucleophile —SH is contacted with an extender comprising a first functionality X that is capable of forming a covalent bond with the reactive nucleophile and a second functionality —SR′″ (where R″ is the same as R″ as defined above) that is capable of forming a disulfide bond. A tether-extender complex is formed which is then contacted with a plurality of ligand candidates. The extender provides one binding determinant (circle) and the ligand candidate provides the second binding determinant (square) and the resulting binding determinants are linked together to form a conjugate compound.

To further illustrate the tethering method using extenders, the method has been applied to a anti-apoptotic target caspase-3, a member of the cysteine aspartyl protease family. There are currently about a dozen known members of the caspase family, many of which are involved in the initiation or propagation of the apoptotic cascade. Caspases are potential drug targets for a variety of therapeutic indications involving excessive or abnormal levels of programmed cell death such as stroke, traumatic brain injury, spinal cord injury, Alzheimer's disease, Huntington's disease, Parkinson's disease, cardiovascular diseases, liver failure, and sepsis. Moreover, caspase-3 includes a naturally occurring cysteine residue at the active site and has been well characterized both functionally and crystallographically.

A suitable extender for use in the caspase-3 active site was designed using the fact i that small aspartyl-based arylacyloxymethyl ketones are known to react irreversibly with the active site cysteine. Two illustrative examples of suitable extenders for use with caspase-3 or other thiol proteases include compounds 13 and 14.

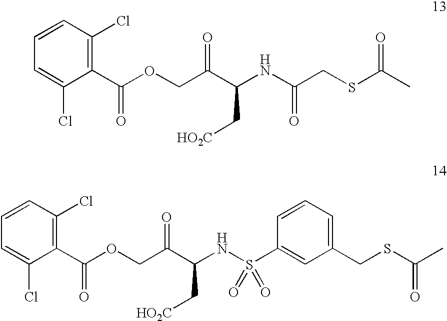

As can be seen, compounds 13 and 14 include an aspartic acid moiety as the binding determinant. Notably, the carbonyl of the aspartic acid moiety is also part of the first functionality (the arylacyloxymethyl ketone moiety) that forms a covalent bond with the thiol of the active site cysteine. Extenders 13 and 14 also include a second functionality, a masked —SH in the form of a thioester that can be unmasked at the appropriate time. For example, the thioester can be converted into the free thiol by treating the target-extender complex with hydroxylamine.

Both extenders were shown to selectively modified caspase-3 at the active site cysteine and were treated with hydroxylamine to generate the following target-extender complexes:

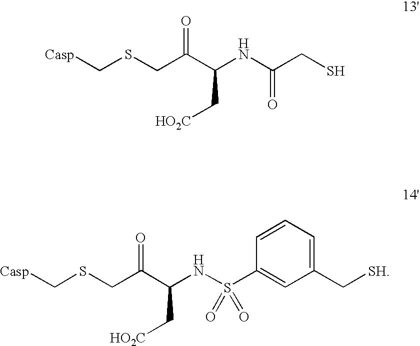

Target-extender complexes 13′ and 14′ were each used in the tethering method against a library of about 10,000 ligand candidates. An illustrative example of a selected ligand-candidate using target-extender complex 13′ is

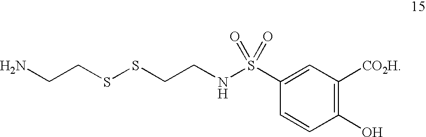

An illustrative example of a selected ligand candidate using target-extender complex 14′ is

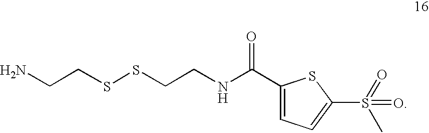

Notably, ligand candidate 15 was not selected by target-extender complex 14′ and ligand candidate 16 was not selected by target-extender complex 13′. Structure-activity relationships among the selected compounds were also evident. For example, ligand candidate 17, 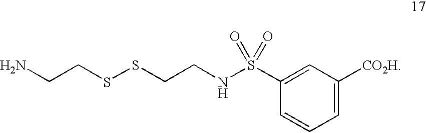
which is identical to ligand candidate 15 except that it lacks a hydroxyl group was not selected by either target-extender complexes 13′ or 14′.

To assess how the extenders and the selected ligand candidates were binding to the target, two structures of the target-extender ligand conjugates were determined. The first structure was of the conjugate that is formed when target-extender complex 13′ is contacted with ligand candidate 15. The second structure was of the conjugate that is formed when target-extender complex 14′ is contacted with ligand candidate 16. Table 3 summarizes selected crystallographic data for these structures.

TABLE 3

| | SPACE | CELL | RES. | COMPLETE- | RYSM | RCRYST | RFREE | #MOLS/ |
|---|---|---|---|---|---|---|---|---|
| DATASET | GROUP | [A,B,C] | [Å] | NESS [%] | [%] | [%] | [%] | AU |
| conjugate | I222 | 69.49 | 20-1.6 | 95.9 | 4.3 | 17.2 | 20.5 | 1 |
| formed from 13 | | 83.60 | | | | | | |
| and 15 | | 95.60 | | | | | | |
| conjugate | P2₁2₁2₁ | 68.85 | 20-2.4 | 95.6 | 10.4 | 24.1 | 29.9 | 2 |
| formed from 14 | | 89.043 | | | | | | |
| and 16 | | 96.5 | | | | | | |

Notably, the aspartic acid moiety of both extenders was superimposable with the aspartyl residue in a known tetrapeptide substrate. With respect to the binding determinant of ligand candidate 15, the salicylate sulfonamide makes numerous contacts with the protein including four hydrogen bonds. The salicylate moiety occupies the P4 pocket of the enzyme that preferentially recognizes aspartic acid in caspase-3. With respect to the binding determinant of ligand candidate 16, the sulfone makes some of the same contacts as the salicylate.

Given that the binding determinants from the extender and the ligand candidates were making productive contacts with the active site of caspase-3, compounds were designed where the disulfides were replaced with more stable linkages. In addition, derivatives were made to probe the SAR of the binding determinants. With respect to the conjugate comprising extender 13 and ligand candidate 15, the target-extender ligand conjugate comprises:

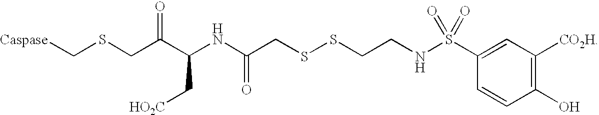

From this conjugate, a class of potent caspase-3 inhibitors was made comprising the moiety

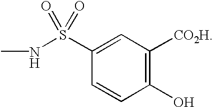

Four illustrative examples of compounds that were made based on the conjugate both for optimization and for SAR are disclosed in Table 4.

TABLE 4

| Compound | | Kᵢ(μM) |
|---|---|---|
| 18 | 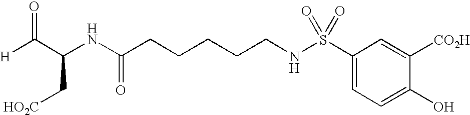 | 2.8 |
| 19 | 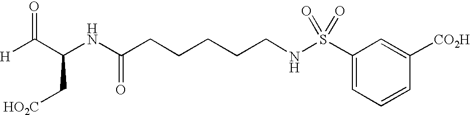 | 15.3 |
| 20 | 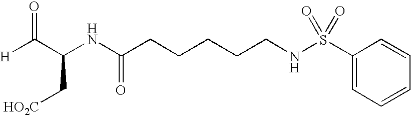 | >100 |
| 21 | 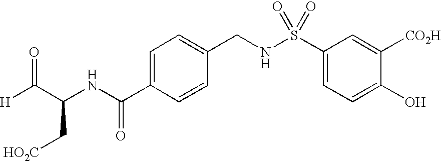 | 0.16 |
| 22 | 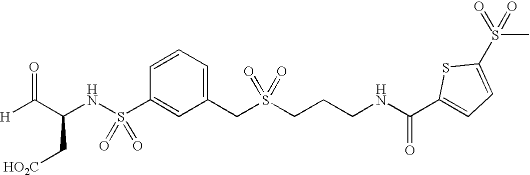 | 0.33 |

As can be seen, a conservative approach was taken wherein the two sulfur atoms were replaced with two methylene units and the arylacyloxymethylketone (first functionality) was replaced with a simple aldehyde resulting in compound 18, a potent inhibitor of caspase-3 with a Kᵢ of 2.8 μM. Removing the hydroxyl group to yield compound 19 reduced the affinity by a factor of five, confirming the SAR observed in the tether screen. Removing both the hydroxyl group and the acid moiety to yield compound 20 ablated binding affinity entirely. Modeling studies suggested that replacing the methylene linker with a rigid aminobenzyl moiety would effectively bridge the distance between the aspartyl group and the salicylate while reducing the entropic costs of the linker. Indeed, as can be seen, compound 21 has a Kᵢ that is more than 10 fold better than compound 18.

Similarly, a novel class of caspase-3 inhibitors resulted from the target-extender ligand conjugate comprising extender 14 and ligand candidate 16,

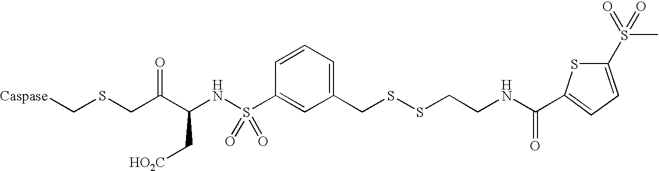

In one embodiment, the compounds comprise the moiety

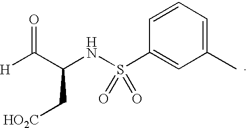

In another embodiment, the compounds are of the structure: 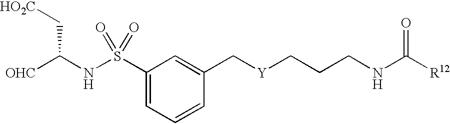
where Y is CH₂, S, SO, SO₂, and R¹² is unsubstituted aryl or substituted aryl. In another embodiment, R¹² is a unsubstituted heteroaryl or substituted heteroaryl. An illustrative example of a compound of this class is compound 22 with a Kᵢ of 0.33 μM.

The salicylate sulfonamide-containing compounds of the present invention are additionally noteworthy. The identification of salicylate sulfonamide as a suitable P4-binding fragment would not have occurred using traditional medicinal chemistry. Using compound 21 as an example, the salicylate sulfonamide-less version of compound 21 inhibits caspase-3 with a Kᵢ of approximately 28 μM. The addition of the salicylate sulfonamide to this fragment improves binding about 200 fold and results in compound 21 that has a Kᵢ of approximately 0.16 μM. In contrast, the binding affinity decreases if one uses a known tripeptide that binds to P1-P3 sites of caspase-3 such as compound I as the starting point.

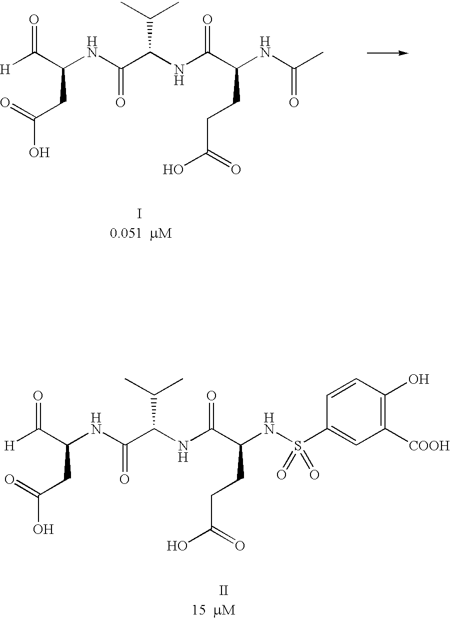

As can be seen compound I has a Kᵢ of 0.051 1M and the addition of the salicylate sulfonamide moiety to this compound yields compound II that shows about a 300 fold decrease in binding affinity. Because of this dramatic decrease, exploring P4 binding with tripeptides would not have resulted in the identification of salicylate sulfonimide as a suitable P4-binding fragment. Yet, compounds that have this fragment available for binding to P4 are potent inhibitors. Consequently, this example highlights the power of tethering to identify important fragments that may not be found using traditional methods. As shown in the case of caspase-3, these fragments can be linked together to form powerful antagonists or agonists of a target of interest.

Another illustration of the power of tethering is the use of tethering to identify and/or optimize small molecule modulators of protein-protein interactions such as those involving interleukin-2 (“IL-2”). Unlike well-defined binding pockets that are typically found in enzymes, protein-protein interactions occur over large amorphous surface areas and are generally intractable to high-throughput screening assays.

IL-2 is a cytokine with a predominant role in the proliferation of activated T helper lymphocytes. Mitogenic stimuli or interaction of the T cell receptor complex with antigen/MHC Be complexes on antigen presenting cells causes synthesis and secretion of IL-2 by the activated T cell, followed by clonal expansion of the antigen-specific cells. These effects are known as autocrine effects. In addition, IL-2 can have paracrine effects on the growth and activity of B cells and natural killer (NK) cells. These outcomes are initiated by interaction of IL-2 with its receptor on the T cell surface. Disruption of the IL-2/IL-2R interaction can suppress immune function, which has a number of clinical indications, including graft vs. host disease (GVHD), transplant rejection, and autoimmune disorders such as psoriasis, uveitis, rheumatoid arthritis, and multiple sclerosis.

Various methods were used to discover a 3 μM (IC₅₀) lead compound 23, 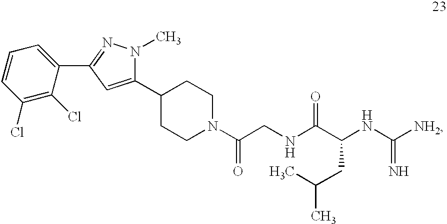
that inhibits the IL-2/IL-2Rα interaction. Traditional methods for further optimization were unsuccessful. Consequently, tethering was used. An x-ray structure of IL-2 bound to a derivative of compound 23 revealed a potential hydrophobic pocket that may provide additional affinity and tethering experiments were performed using two cysteine mutants of IL-2, Y31C and L72C, that were made to explore this site.

These tethering experiments identified several fragments that bind to the adjacent hydrophobic pocket including those below: 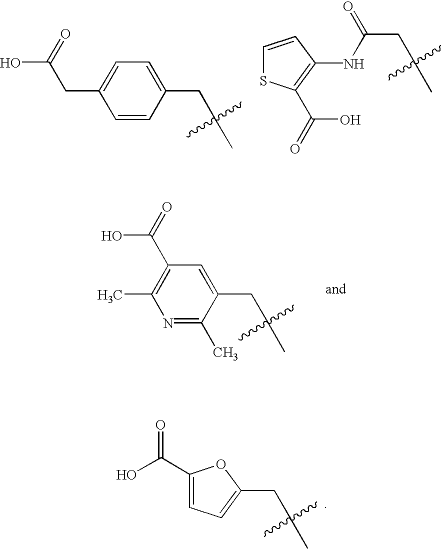
The identified binding determinants were then merged onto compound 23 resulting in compounds with improved binding affinities. The best compound was compound 24 whose structure is shown below 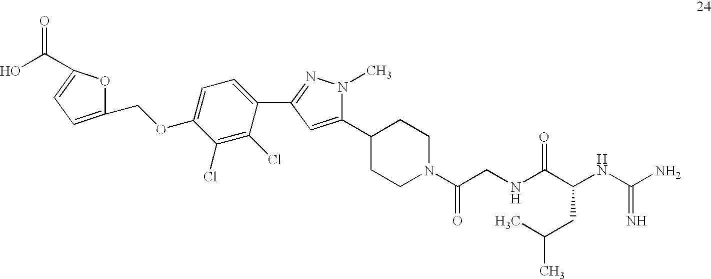
that inhibited theIL-2/IL2Rα interaction with an IC₅₀ of 65 nM, an over 45 fold improvement over compound 23. This example highlights how tethering can be used to identify/optimize compounds against targets that were traditionally intractable to high throughput screening.

As can be seen in FIG. 2, the compound bound to the target can be readily detected and identified by mass spectroscopy (“MS”). MS detects molecules based on mass-to-charge ratio (m/z) and can resolve molecules based on their sizes (reviewed in Yates, Trends Genet. 16: 5-8 [2000]). The target-compound conjugate can be detected directly in the MS or the target compound conjugate can be fragmented prior to detection. Alternatively, the compound can be liberated within the mass spectrophotometer and subsequently identified. Moreover, MS can be used alone or in combination with other means for detection or identifying the compounds covalently bound to the target. Further descriptions of mass spectroscopy techniques include Fitzgerald and Siuzdak, Chemistry & Biology 3: 707-715 [1996]; Chu et al., J. Am. Chem. Soc. 118: 7827-7835 [1996]; Siudzak, Proc. Natl. Acad. Sci. USA 91: 11290-11297 [1994]; Burlingame et al., Anal. Chem. 68: 599R-651R [1996]; Wu et al., Chemistry & Biology 4: 653-657 [1997]; and Loo et al., Am. Reports Med. Chem. 31: 319-325 [1996]).

Alternatively, the target-compound conjugate can be identified using other means. For example, one can employ various chromatographic techniques such as liquid chromatography, thin layer chromatography and the like for separation of the components of the reaction mixture so as to enhance the ability to identify the covalently bound molecule. Such chromatographic techniques can be employed in combination with mass spectroscopy or separate from mass spectroscopy. One can also couple a labeled probe (fluorescently, radioactively, or otherwise) to the liberated compound so as to facilitate its identification using any of the above techniques. In yet another embodiment, the formation of the new bonds liberates a labeled probe, which can then be monitored. A simple functional assay, such as an ELISA or enzymatic assay can also be used to detect binding when binding occurs in an area essential for what the assay measures. Other techniques that may find use for identifying the organic compound bound to the target molecule include, for example, nuclear magnetic resonance (NMR), surface plasmon resonance (e.g., BIACORE), capillary electrophoresis, X-ray crystallography, and the like, all of which will be well known to those skilled in the art.

The methods described herein provide powerful techniques for generating drug leads, and allowing the identification of one or more fragments that bind weakly, or with moderate binding affinity, to a target at sites near one another, and the synthesis of diaphores or larger molecules comprising the identified fragments (monophores) covalently linked to each other to produce higher affinity compounds. The monophores, diaphores or similar multimeric compounds including further ligand compounds, are valuable tools in rational drug design, which can be further modified and optimized using medicinal chemistry approaches and structure-aided design.

Clearly, the monophores or multiphores identified in accordance with the present invention and the modified drug leads and drugs designed therefrom can be used, for example, to regulate a variety of in vitro and in vivo biological processes which require or depend on the site-specific interaction of two molecules. Molecules which bind to a polynucleotide can be used, for example, to inhibit or prevent gene activation by blocking the access of a factor needed for activation to the target gene, or repress transcription by stabilizing duplex DNA or interfering with the transcriptional machinery.

### Equivalents

The representative examples that follow are intended to help illustrate the invention, and are not intended to, nor should they be construed to, limit the scope of the invention. Indeed, various modifications of the invention and many further embodiments thereof, in addition to A those, shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Exemplification

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

1) General Description of Synthetic Strategy:

As described generally above, exemplary compounds and libraries of compounds are synthesized by coupling appropriate amine, carboxylic acid, sulfonyl chloride, etc. building blocks with appropriate linkers. Described in more detail below is the synthesis of exemplary linkers and exemplary compounds and libraries of compounds.

A. Synthesis of Exemplary Linkers:

1. Amine Linker

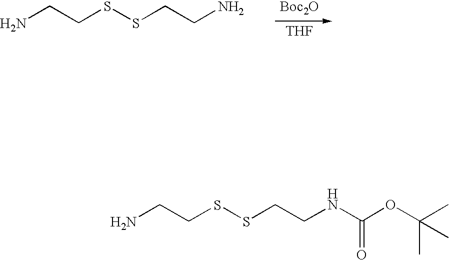

To cystamine dihydrochloride (100 g, 444 mmol) was added 5 N NaOH (400 mL) and the suspension stirred until a clear solution formed. The solution was extracted with DCM (6×200 mL) and the combined DCM layers dried (Na₂SO₄), filtered and concentrated to afford 64.5 g of the desired free base (95%).

To a solution of the free base (422 mmol) in THF (285 mL) was added dropwise a solution of di-t-butyldicarbonate (0.5 eq, 212 mmol) in THF (212 mL). The reaction was allowed to stir overnight, then concentrated to an oil, taken up in 1 M NaHSO₄ (500 mL), and washed with ethylacetate. The aqueous layer was cooled in an ice-bath, treated with 5 M NaOH (200 mL), and the resulting solution immediate washed with DCM. The DCM layers were combined, dried (Na₂SO₄), filtered and concentrated to afford 11.4 g of the desired mono-Boc cystamine (21%).

2. Carboxylate Linker

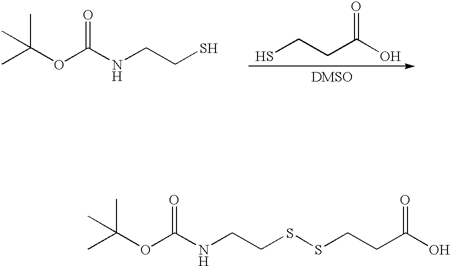

To tert-butyl N-(2-mercaptoethyl)carbamate (10 g, 56 mmol) in DMSO (20 mL) was added 3-mercaptopropionic acid (6 g, 57 mmol) and the solution heated at 70° C. for 48 hours. The solution was cooled, and the resulting waxy solid dissolved in chloroform (200 mL) and washed with 5% aqueous NaHCO₃ (4×50 mL). The aqueous layers were combined, carefully acidified to litmus with 1 N HCl, and washed with CHCl₃ (4×50 mL). The organic layers were combined, washed with brine, dried (Na₂SO₄), concentrated and then purified on silica gel (9/1 DCM/MeOH) to afford 1.8 g of a colorless oil (12%).

3. Synthesis of the Alkoxyamine Linker:

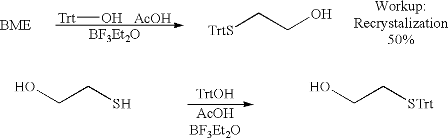

Dissolve 1 eq. B-mercaptoethanol in AcOH. Add 1 eq. of trityl alcohol and heat until dissolved. Add 1 eq of BF₃ET₂O. After 10 min. quench the reaction with H₂O. Conc. in vacuo. Dilute into dichloromethane and wash 3× with 3N NaOH, with brine, and dry on sodium sulfate. Rotovap and recrystalize from EtOAc/Hexanes.

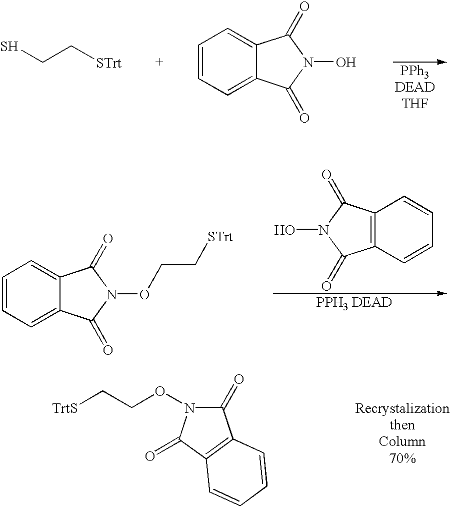

Under N₂ combine 2 eq. PPh₃ and 2 eq. N-hydroxy phthalamide and dissolve in THF. Cool on ice/NaCl/isopropanol to −10° C. Add 2 eq. of diethyl azodicarboxylate via syringe over 1 min. Solution turns dark color. Wait 1 min. Add trityl protected B-mercaptoethanol as a solution in THF. React for 2 hours then let slowly warm to r.t. Remove solvent. Dissolve in minimum EtOAc. Remove ppt. via filtration. Remove solvent and chromatograph: Gradient to 7:1 then back down to 3:1 hex:etoac.

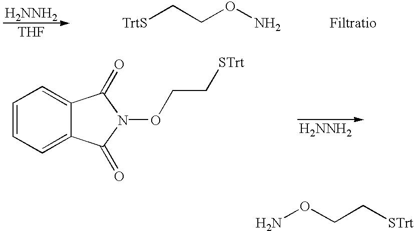

Dissolve phthalimide in THF. Add excess hydrazine (anhydrous. in THF). Ppt forms within 15 min. Let stir additional 30-40 min. Add 2× vol. 7:1 Hex:EtOAc. Filter through glass wool. R-Vap. Dissolve in min. etoac. Add 7:1 and filter again. Remove solvent and dry under vacuum.

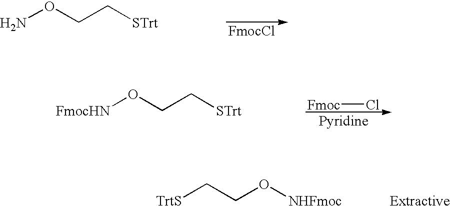

Dissolve alkoxyamine in THF under N₂. Add 1.5 eq. of pyridine via syringe. Add solution of 9-Fluorenylmethyl chloroformate in THF. Rxn. ppt. during addition. Stir 20 min. Add ether (2× total volume of THF). Wash two times with 10% citric acid. Wash once with brine. Dry on sodium sulfate, then remove solvent and dry under vacuum

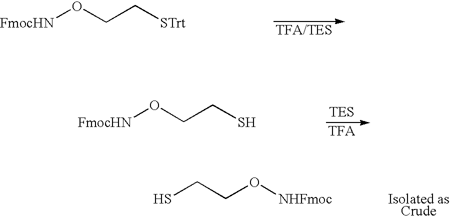

Dissolve trityl thiol in DCM. Add triethylsilane followed by TFA and monitor by TLC. When complete remove solvent and then coevaporate 3× with DCM. 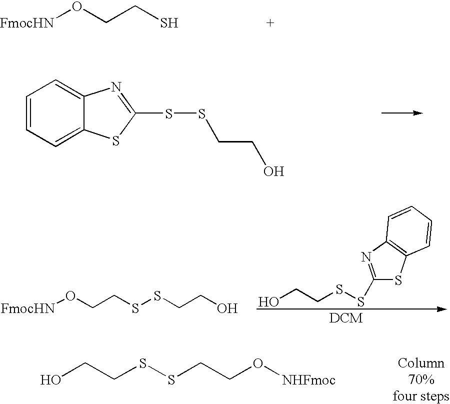
Dissolve thiol in DCM. Add 1 eq of activated disulfide. Stir 30 min. TLC 1:1 Hex:EtOAc. Chrom. 4:1 to 2:1 to 1:1.

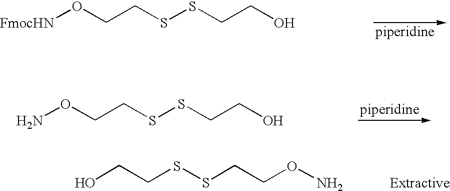

Dissolve Fmoc protected alkoxyamine in THF. Add piperidine (100 miroliters for 40 mgs.). Stir 5-15 min. Remove solvent, then triturate with hexanes to remove fulvene by-product. Dry under vacuum and store product as a 100 mM stock in methanol. See notebooks 23 and 41 for more detailed protocols and NMR.

4. Synthesis of Bromoacetamide Linker: 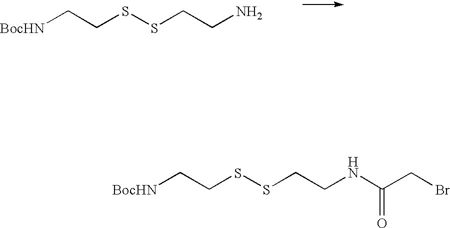
Dissolve 1 eq. of bromoacetic acid in a small amount of ether. Chill on ice. Add 1 eq. of isobutyl chloroformate and 1 eq. of N-methyl morpholine. Remove N-methyl morpholine HCl precipitate by filtration into a flask cooled to 0C and wash with ether. Add 1 eq. of mono-Boc protected cystamine as a 1M solution in DCM. Monitor reaction by TLC, upon completion remove solvent and chromatograph with 2:1 hexanes/ethyl acetate.

5. Synthesis of MTSPA

Sodium Methanesulfinate (tech grade, 85%) Aldrich 43,306-310 g for $50 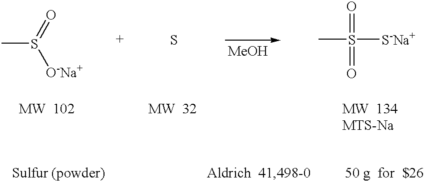
2 g Sodium Methanesulfinate (MW 102, 17 mmol based on 85% purity)
0.55g Sulfur (MW 32, 17 mmol)
(JOC 53 1988 p.401)

Combine Sodium Methanesulfinate and Sulfur in 60 ML on MeOH (reagent grade) and heat to reflux. Reflux for 1 hour, at which point the sulfur will have dissolved to yield a hazy solution. Let cool to room temperature and filter through Celite. Remove methanol in vacuo, and recrystallize from approximately 50 mL hot EtOH. For the recrystallization, there will be some insoluble material that must be removed by hot filtration through celite. Isolate approximately 1.4 g, 60%, from first crop, second crop possible. 1H NMR: singlet at 3.36 ppm (D20). Used internal std. to confirm that all sodium bromide has been removed.

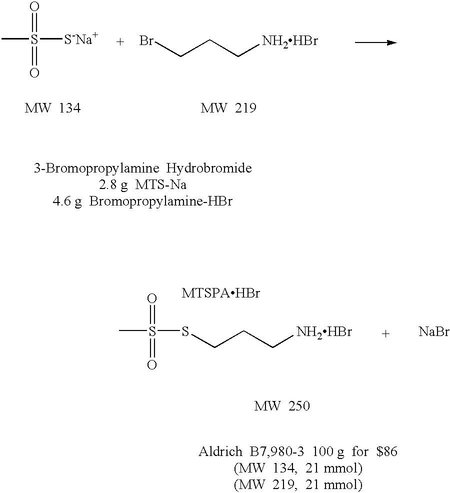

Combine reagents in 40 mL of EtOH and heat at reflux for 6 hours (this is probably complete much faster than 6 hours). Let cool, filter to remove NaBr and wash with cold EtOH (Caution: product may crystallize out with sodium bromide). Concentrate filtrate, recrystallize from EtOH. Isolate approximately 60% (unoptimized).

6. Synthesis of Thiopropylamine Linker
N-Boc aminoethanethiol Fluka 15303

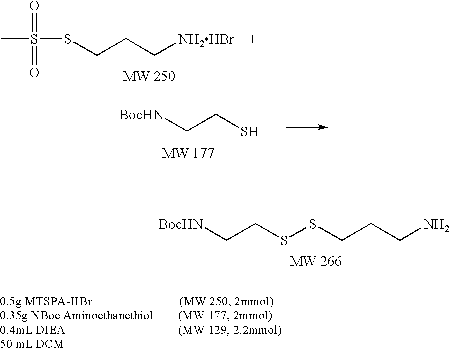

Dissolve MTSPA in 40 mL of DCM with 0.4 mL DIEA (will dissolve slowly, sonication helps. Slight insoluble haze may be trace of NaBr from previous step). Dissolve N-Boc aminoethane thiol in 10 mL DCM and add dropwise over 5 minutes to the stirred solution of MTSPA. Check by TLC after 10 minutes (5% MeOH in DCM with a few drops of TEA) to see a single spot, RF 0.3, with slight UV activity and strong ninhydrin response.

Filter reaction through Celite to remove insoluble materials. Remove solvent in vacuo and dissolve residue in 5 mL of 1M NaHSO₄. Wash twice with 10 mL EtOAc, then cool aqueous portion on ice and raise pH to 11 with 5M NaOH. Extract twice with 10 mL DCM, wash organics with 10 mL brine and then dry organics with Na2SO4. Concentrate and dry under vacuum, isolate approximately 85% yield colorless oil.

B. General Description of Syntheses of Exemplary Classes of Compounds and Libraries of Compounds:

1. Carboxylic Acid Derived Monophores

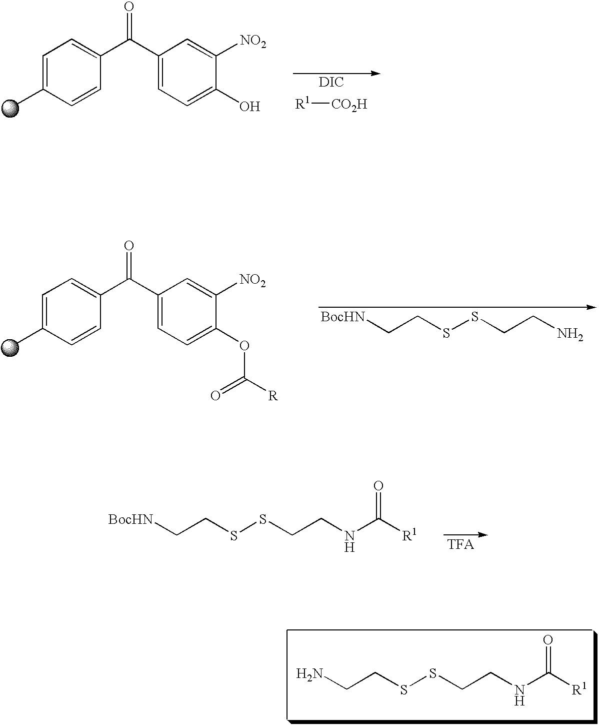

Synthesis of acid derived disulfide library: 260 μMols of 594 carboxylic acids were acylated in parallel with 130 μMol equivalents of 4-hydroxy-3-nitro-benzophenone on polystyrene using DIC in DMF. After 4 hours at room temperature, the resin was rinsed with DMF (2×), DCM (3×), and THF (1×) to remove uncoupled acid and DIC. The acids were cleaved from the resin via amide formation with 66 μMols of mono-boc protected cystamine in THF. After reaction for 12 hours at room temperature, the solvent was evaporated and the boc group was removed from the uncoupled half of each disulfide using 80% TFA in DCM. 530 (89%) acid derived disulfides passed Q.C. by LCMS.

2. Isocyanate and Thioisocyanate Derived Monophores.

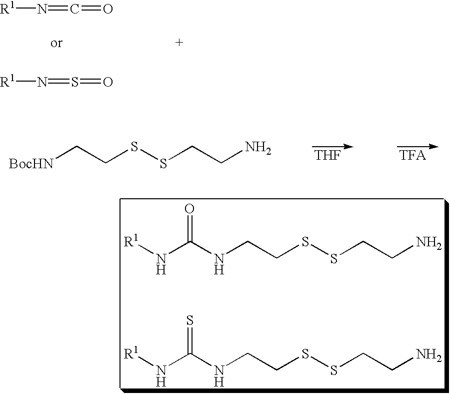

10 μMols of 64 isocyanates and 120 isothiocyanates were coupled in parallel with 10.5 μMols of mono-boc protected cystamine in THF. After reaction for 12 hours at room temperature, the solvent was evaporated and the boc group was removed from the uncoupled half of each disulfide using 50% TFA in DCM. 58 (91%) isocyanate derived disulfides and 94 (78%) isothiocyanate derived disulfides passed Q.C. by LCMS.

3. Sulfonyl Chloride Derived Monophores

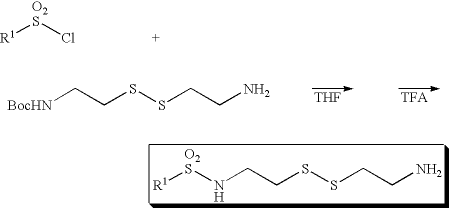

10 μMols of 66 sulfonyl chlorides were coupled with 10.5 μMols of mono-boc protected cystamine in THF (2% diisopropyl ethyl amine) in the presence of 15 milligrams of poly(4-vinyl chloride). After 48 hours the poly(4-vinyl chloride) was removed via filtration and the solvent was evaporated. The boc group was removed from the uncoupled half of each disulfide using 50% TFA in DCM. 60 (91%) sulfonyl chloride derived disulfides passed Q.C. by LCMS.

4. Aldehyde and Ketone Derived Monophores.

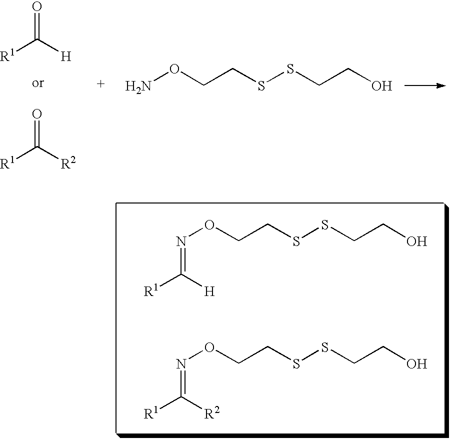

Synthesis of ketone and aldehyde derived disulfide libraries: 10 μmols of 259 aldehydes and 225 ketones were coupled in parallel with 10.5 μMols of HO(CH₂)₂SS(CH₂)₂ONH₂ in 1:1 methanol:chloroform (2% AcOH) for 12 hours at room temperature to yield the oxime product. 259 (100%) aldehyde disulfides and 189 (84%) ketone derived disulfides passed Q.C. by LCMS.

5. Phenol Derived Monophores

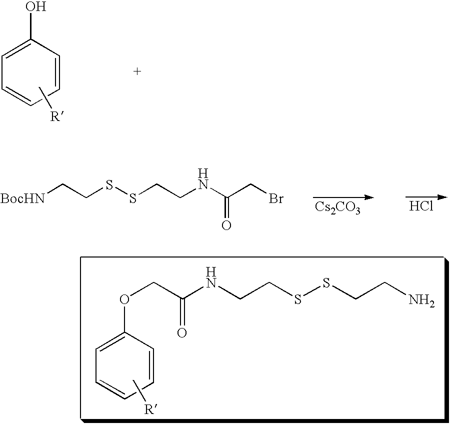

Synthesis of phenol derived libraries: 10 μmol each of 206 phenols were dissolved in 0.5 mL DMF. An aqueous solution of 0.8 M Cs₂CO₃ (12.51L) was added followed by a solution of the 10,mol of the bromoacetamide linker in 12.5 μL DMF. Reactions were sealed and heated at 40° C. for 15 hrs. Products were isolated by diluting reactions with 2 mL DCM, washing with 1 mL 1M NaOH, washing with brine and drying over sodium sulfate. The Boc protecting group was removed by addition of 2M HCl in ether and the HCL salts of the amines were obtained after evaporation of solvents.

6. Synthesis of Methylthiosulfonate Analogs (MTS)

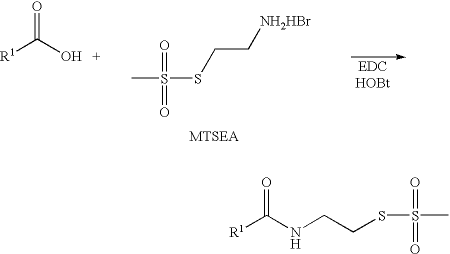

Dissolve methyl thiosulfonate ethyl amine (0.25 mmol, 59 mg) (synthesized in the same manner as MTSPA, described above, or purchased from Toronto Research Chemicals) in 4 mL dichloromethane with 2 equivalents of diisopropylethyl amine. In a separate vial, combine 0.25 mmol of the carboxylic acid, 0.3 mmol of EDC and 0.3 mmol of HOBt. Add the solution of MTSEA and DIEA in DCM to the mixture of carboxylic acid with EDC and HOBt and stir. Monitor by HPLC, the coupling reaction is typically complete within 2 hrs. To isolate product first wash the organic solution with water, then with 1M aqueous NaHSO₄ then with brine. Dry the organic phase with sodium sulfate and remove solvent by rotary evaporation. Products can be further purified by reverse phase preparative HPLC.

C. Generation of Building Block Diversity:

As discussed above, a variety of building blocks can be used to generate the tethering reagents of the invention. For example, a number of commercially available bifunctional amino acids, as shown directly below, are available for use in the present invention. It will be appreciated, however, that the building blocks to be used in the invention are not limited to these particular reagents. Additionally, these commerically available reagents can be subsequently modified to generate “customized” reagents.

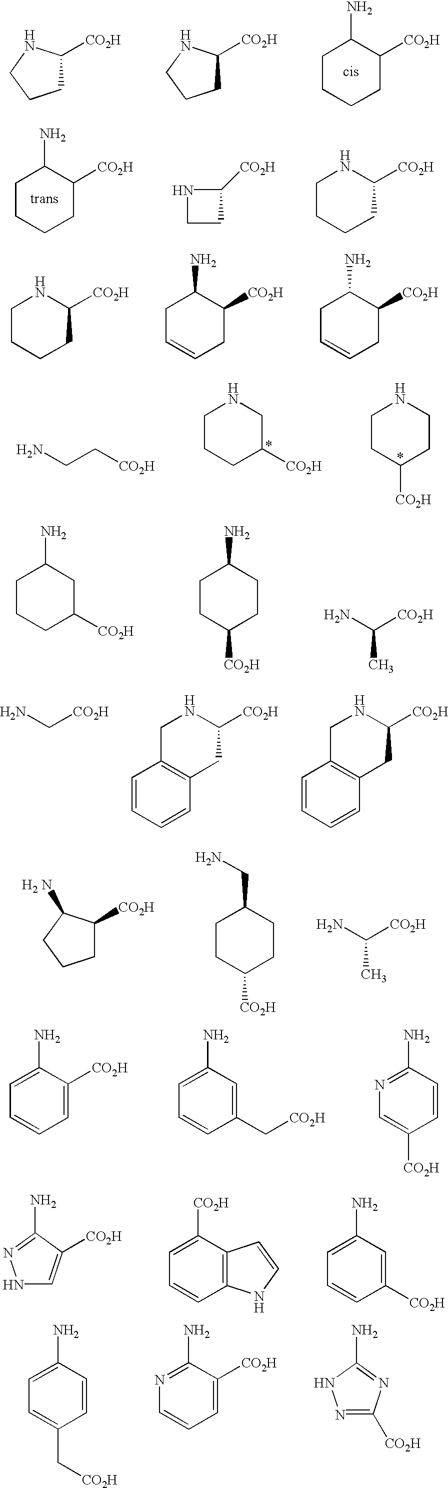

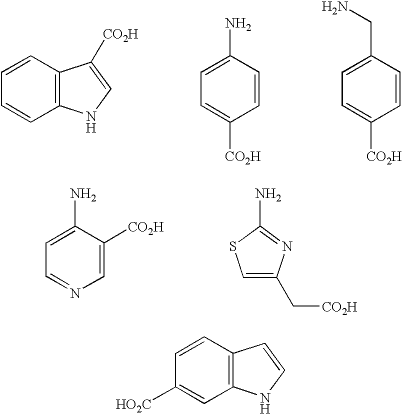

Although a variety of inventive tethering reagents and libraries of reagents can be prepared using commercially available building blocks, it is also possible to “customize” these building blocks, or alternatively, develop building blocks for the development of further “customized” tethering reagents.

As but one example for the possibility of diversification, the addition of even a single additional synthetic step prior to the installation of the tether or “nub” (“1+Nub”) can dramatically increase the number of new compounds accessible from even simple starting materials. Even multistep syntheses can be considered, provided the diversity element is installed in the penultimate step. Examples of such “1+nub,” “2+nub,” etc. syntheses starting from L-proline are illustrated in one embodiment, as shown directly below:

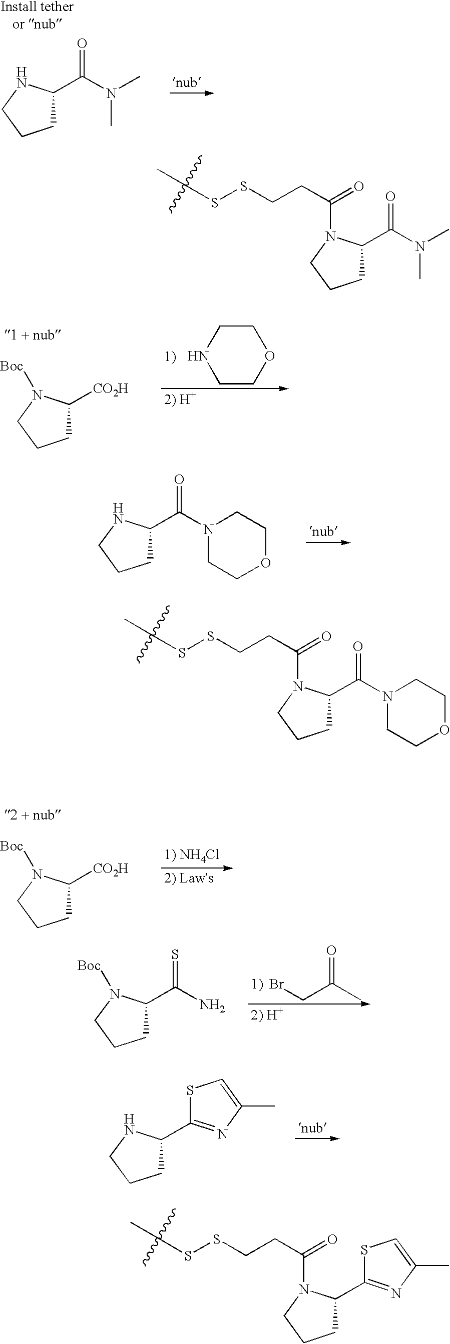

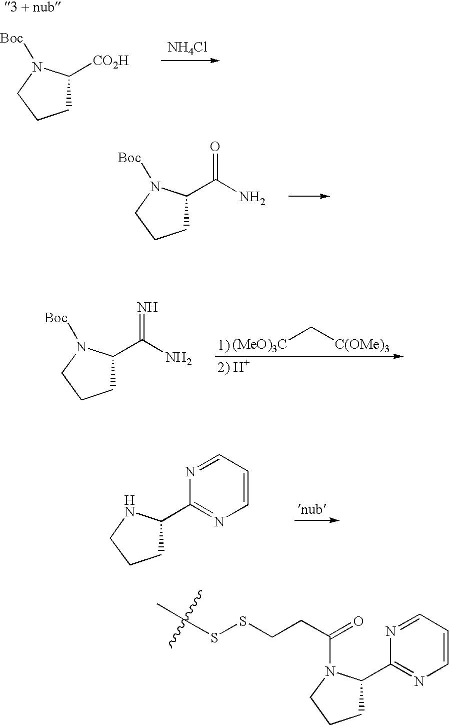

It will be appreciated that the example of the constrained amino acid described above can be further modified (for example via C- or N-side modifications as described in more detail herein) to generate additional diversity in the tethering reagents and libraries described herein. Constrained amino acids in certain embodiments are utilized for their precedence in biologically active molecules and theoretical considerations (fewer rotational degrees of freedom, resist hydrophobic collapse, positional and stereochemical isomers can sample different regions of conformational space, etc.). A general schematic for the N- and C-side modification of a constrained amino acid is illustrated directly below:

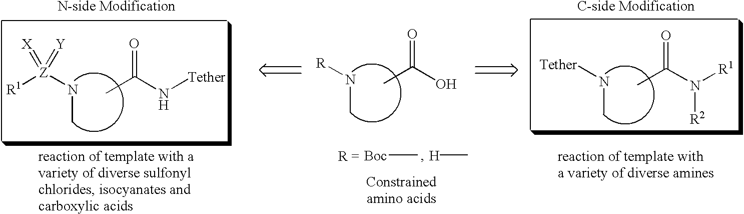

Exemplary constrained amino acid blocks include, but are not limited to:

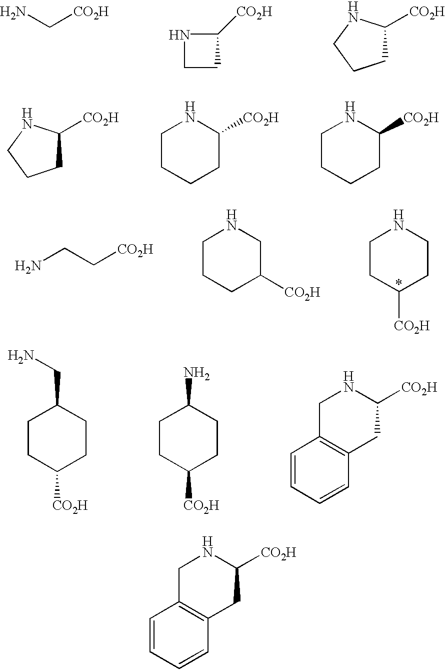

Trifunctional building blocks were also considered advantageous, since the additional point of modification can allow 1) the synthesis of additional regioisomers, 2) combinatorial elaboration/refinement of a monophore hit, and 3) a potential site for recombination with other monophore hits. The latter point may have particular utility with tethering, since hits obtained from different Cys mutants will by definition have their recombination nubs improperly oriented. Few constrained trifunctional building blocks are commercially available. The reagents trans-hydroxyproline, and R- and S-piperazine-2-carboxylic acid were available, and this list was supplemented with the unconstrained amino acids D- and L-2,3-diaminopropionic acid (DAP), Asn, Gin, and Tyr as illustrated in the figure, below.

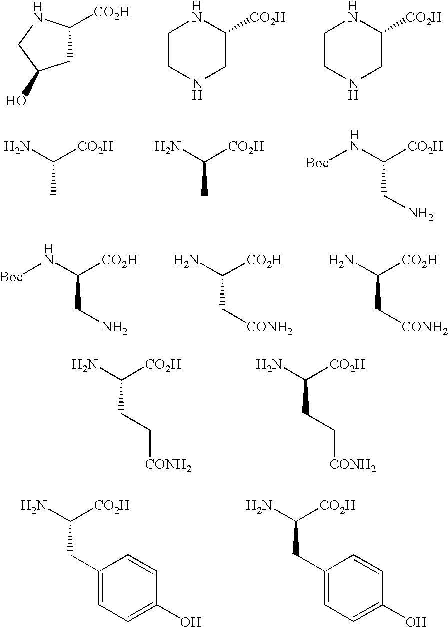

1. N-Side Modifications

Selection of Reagents for “N-Side” Modifications.

Both the N-terminal and C-terminal sides of a constrained amino acid can be employed for the incorporation of diversity elements. Approximately 200 isocyanates and 100 sulfonylchlorides are available in reasonable quantity commercially, and these sets can be readily examined by simple inspection to select reagents. Just over 250 carboxylic acids were selected.

Exemplary Core Scaffolds.

Many constrained amino acid scaffolds were converted into common intermediates for tethering libraries using the scheme illustrated below. Most of these were prepared in 25 mmol quantity, which is sufficient for all 250 planned N-side modifications.

Scaffold Synthesis Scheme:

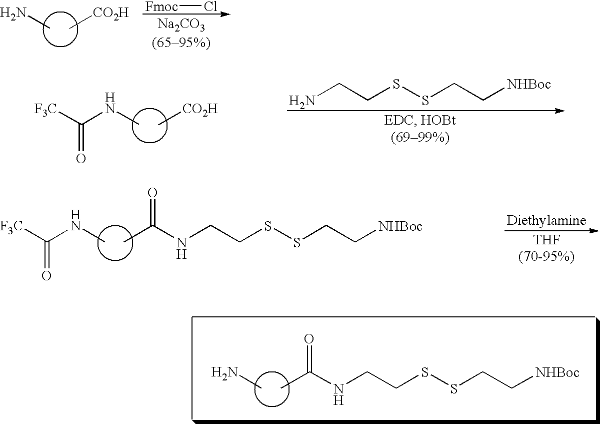

Scaffolds Synthesized for First 1+Nub Libraries.

Shown below are examples of exemplary core scaffolds prepared in sufficient quantity for library synthesis. In most cases, these products were purified to homogeneity by flash chromatography prior to library synthesis.

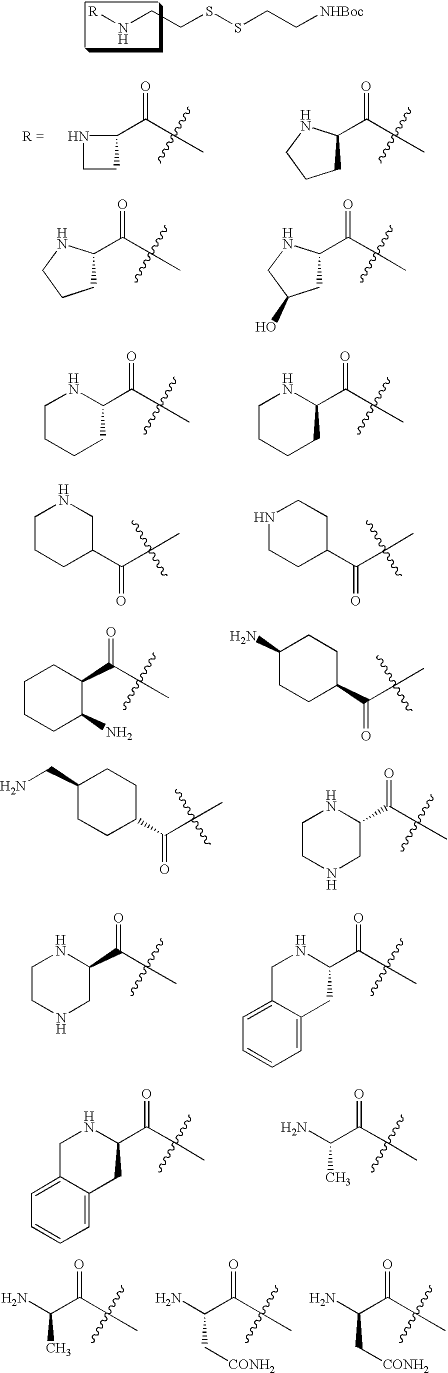

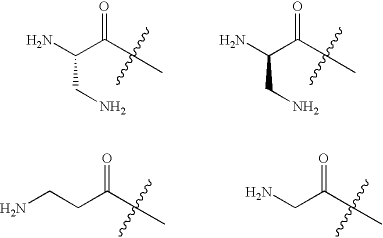

Library Preparation: Synthesis Protocols.

As each scaffold is prepared it will be modified in the same fashion with the same set of building blocks. There is significant efficiency gained in this process, since SOPs developed for the first set of scaffolds can be used in subsequent experiments without modification. The Tecan was programmed in several different configurations before a satisfactory arrangement was found. This method accommodates up to 66 N-side diversity elements and 2 core scaffolds on the deck at one time. There is one quadrant that is not occupied by starting materials and is the only point where common reagents are added.

All core scaffolds were modified with the N-side diversity inputs to prepare well over * 5,000 new monophores. Reactions were performed using EDC/HOBt chemistry in 8:1 DCM/DMF.

Library Purification:

An efficient liquid-liquid extraction procedure suitable for semi-automation on a Tecan robotic workstation was devised. A program specific for the 1+Nub chemistry was developed, and is shown schematically, below. In this method, crude reaction products (in 8/1 DCM/DMF) are first treated with 1 mL of 0.25 M aqueous HCl. The vials are then vigorously stirred on a vortex shaker to completely intermix the aqueous and organic layers.

The vial is allowed to stand, and then the organic (bottom) layer is transferred to a new vial. This solution is then treated with saturated aqueous sodium bicarbonate, and the agitation procedure repeated. A 24-well deep well filter plate is then charged with anhydrous Mg₂SO₄ and placed over a rack of 24 tared, bar-coded vials. The final organic layer is dispensed into the filter plate and allowed to drip into the tared vials. A 1 mL DCM wash is added to the filter plate, and the combined filtrates are evaporated to dryness to complete the semi-automated work-up. Boc protection on the cystamine linker is removed with HCl/Dioxane and the vials concentrated to dryness again. All library members are characterized by LCMS; in some cases approximately 10% of the library is also analyzed by ¹H NMR. With hydrophobic monophores, this method removes most of the reagents and failure products and affords good recovery of the desired product. Hydrophilic monophores and monophores with ionizable functional can require HPLC purification as some are removed in the extraction process. Regardless, the liquid-liquid extraction method is suitable for the majority of the compounds prepared.

2. “C-Side” Libraries

“C-Side” Modifications.

C-side modifications consist of the condensation of a highly diverse set of amines with conformationally-constrained core scaffolds bearing free carboxylic acids (see below). The chosen amines comprise 293 inputs that were selected based upon the diversity of functionality that they display.

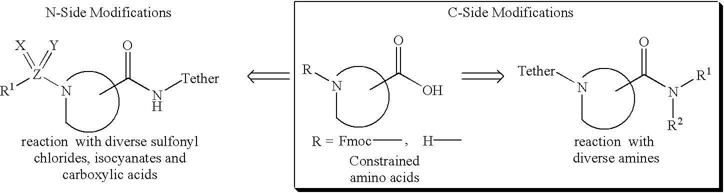

Scaffold Synthesis.

A procedure was devised that permits the synthesis of C-side core scaffolds in the absence of protecting group chemistry, eliminating as much as three synthetic steps. As shown in the following scheme, the carboxylic acid tethering linker is converted to its acyl chloride with Vilsmeier reagent, and then added to an ice-cold suspension of excess amino acid in DCM/TEA. This procedure worked for most of the constrained amino acids.

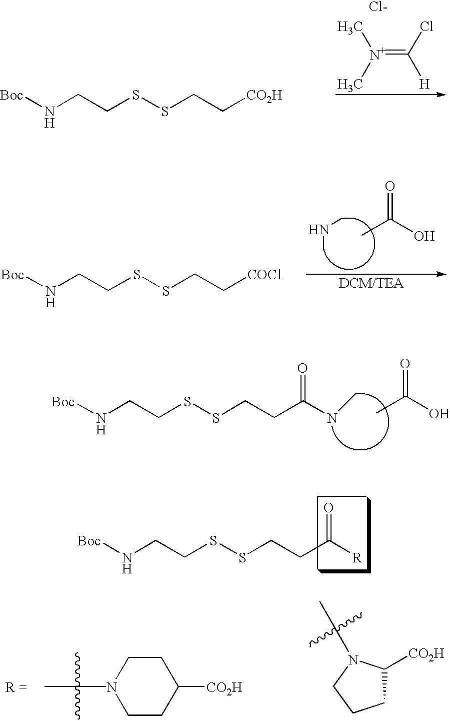

Shown in the figure below are the core scaffolds that were prepared for C-side libraries:

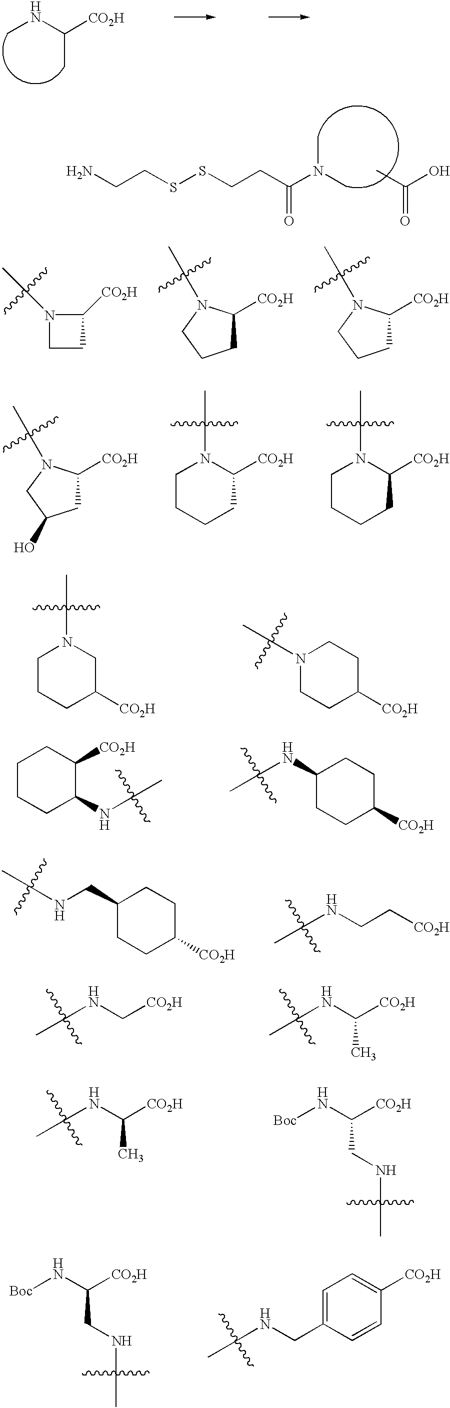

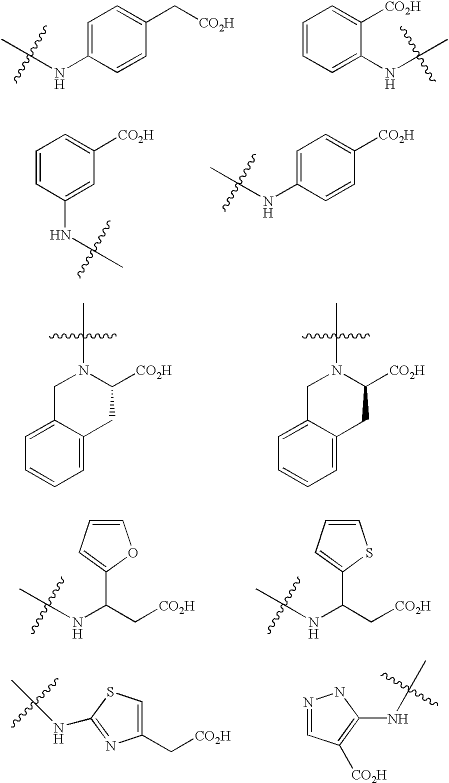

Exemplary Amine Reacations.

Many of the amines we wished to condense with the above scaffolds contain free hydroxyls, carboxylates, and other functionality that can afford undesired side-products if the amine were simply coupled to a core scaffold using a conventional activating agent. Alternatively, a preformed active ester can often react preferentially with the desired amine and thus minimize side-product formation. Pentafluorophenyl (pFp) esters were first tried since they are often isolated as crystalline solids yet are quite reactive. In model reactions, a representative -OpFp ester was used to acylate a cross-section of amines. Although, products were found, many reactions were incomplete (even after 24 h). Addition of pyridine, DMAP, etc. had only marginal impact on product yields. Alternatively, activation of the acid with Vilsmeier reagent followed by treatment with the same amine set led to a good conversion of products in most cases. All the amines were readily converted to products except for the indicated aniline as shown in the figure, below. All the C-side libraries were prepared using the Vilsmeier chemistry.

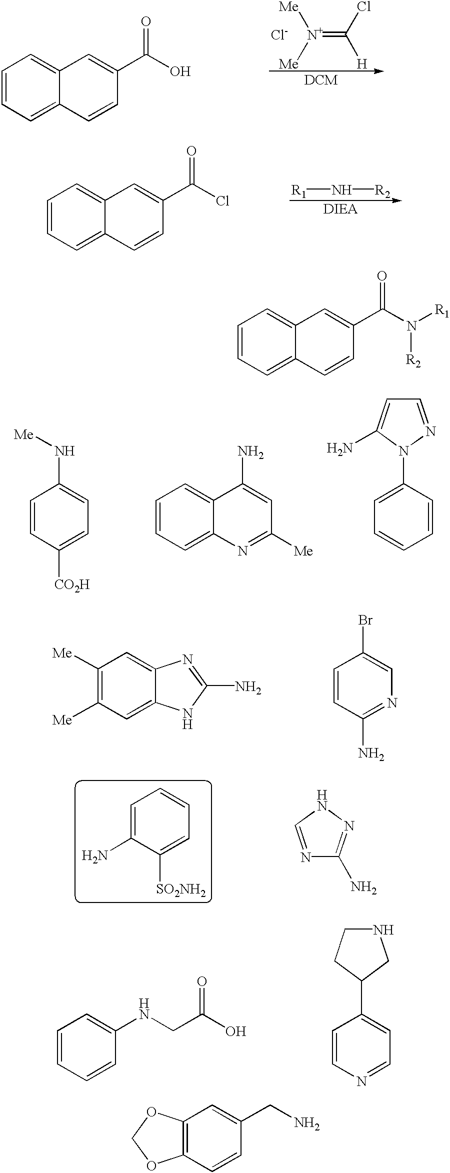

3. Other Diversified Scaffolds:

As described above, it is also possible to use additional diversified building blocks for the tethering reagents of the invention. For example, motifs that occur frequently and are well represented in a cross-section of therapeutic areas are heterocycles containing one or two heteroatoms, such as pyridines, thiazoles, oxazoles, pryimidines, etc. Another ubiquitous motif was tertiary amines. Exemplary syntheses for these fragments of interest is described in more detail below.

Synthesis of Heterocycles

As much as possible, chemistries are chosen that are flexible such that simple variations can afford more than one class of building block. Carboxylic acids are common synthons for the synthesis of heterocycles, and simple derivatives of this functional group can be combined with an electrophile to create a heterocycle. This is shown schematically, below:

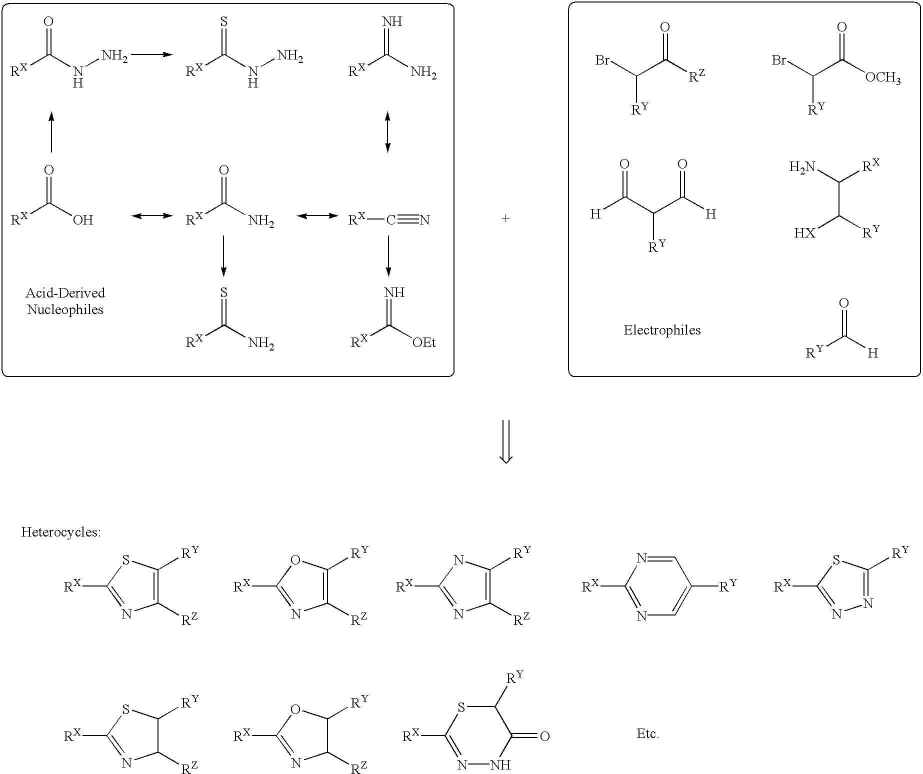

These heterocycles are prepared as building blocks for subsequent derivatization with other diversity elements. Alternatively, the chemistries shown above can be used to make many subtle variations of each heterocycle as exemplified below and herein.

Synthesis of Thiazoles.

A modified Hantzsch procedure has been employed in the synthesis of several thiazoles. The thiazoles were largely designed based upon the most common form of appearance of this motif in the MDDR. Appropriate amino acids were converted to thioamides in two steps, followed by cyclodehydration with the appropriate bromoketone:

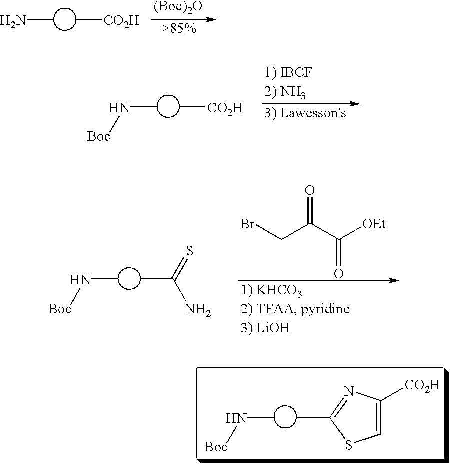

Several thiazole amino acid derivatives were prepared, encompassing a cross-section of conformational constraint (see below), and these were used to prepare a library as described in the working examples.

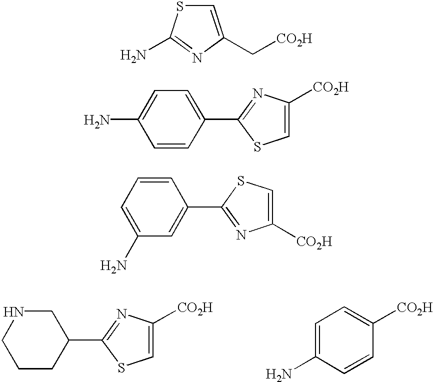

Synthesis of Pyridones and Pyrrolidinones.

Using aza-annulation chemistry, a common intermediate was employed for the synthesis of two piperidones and a pyrrolidone in good yield (see below). This chemistry is sufficiently flexible to permit the synthesis of bicyclic analogs of these motifs, some of which are recognized beta-turn mimetics. During the optimization of the chemistry it was found that some protecting group manipulations (ester hydrolysis) led to the formation of significant by-products derived from the disulfide of the tether linker. The optimized route used O-allyl protection, which could be efficiently deprotected in the presence of the disulfide using Pd(PPh₃)₄. These were used to prepare “C-side” libraries as described previously.

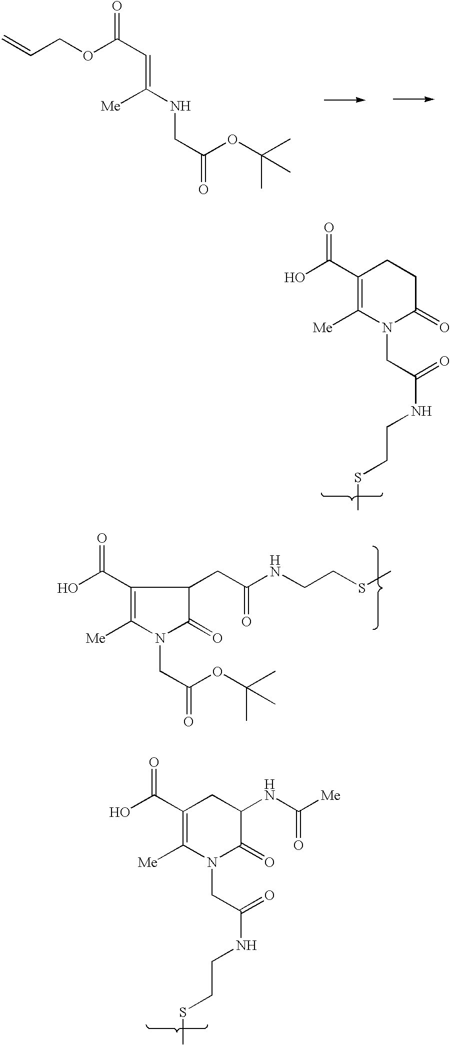

Substituted Piperazines.

Piperazines are the most common motif in the CMC and MDDR, and several N-side Nub+l libraries have already been prepared from piperazine scaffolds. Shown below is a common intermediate that can be used in the preparation of three piperazine motifs (and their regioisomers), including forms which will ultimately display a basic amine (Boc-protected), a tertiary amine (N-methyl) and an amide (N-acetyl). These three motifs represent fragments of the most common forms of derivatization for this core scaffold. Each of these can be made from the indicated Boc/Fmoc intermediate. After much experimentation, we have devised an efficient two step procedure for the preparation of this intermediate, and over 50 g are currently in-house. Each piperazine motif will be systematically prepared and derivatized using the “Go To” amine set.

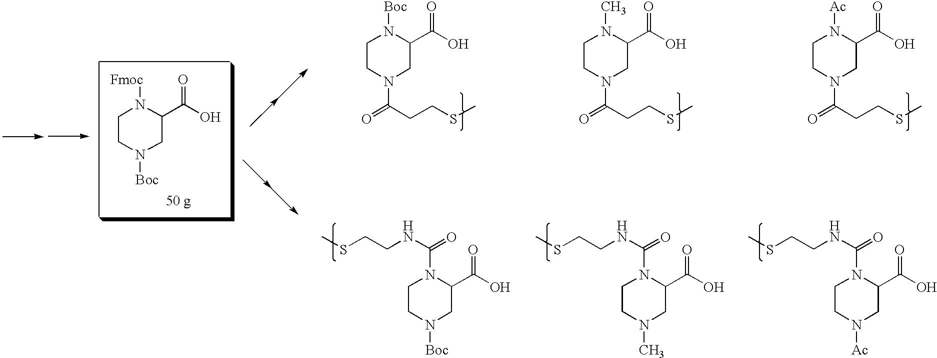

Oxazoles.

Oxazoles are also a common motif. A variety of oxazoles were prepared from conformationally constrained amino acids and serine using the route shown below:

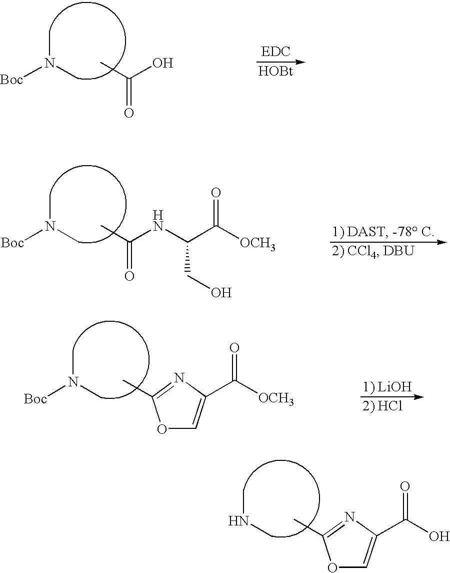

The following scaffolds were synthesized:

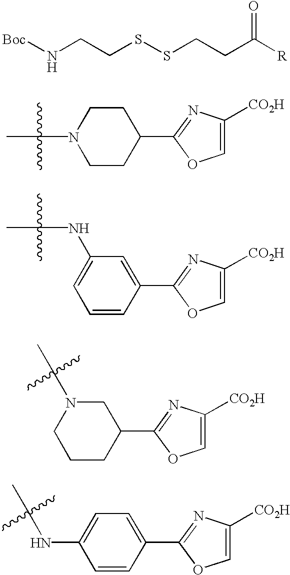

These intermediates are converted to tethering monophores using a route similar to that previously described for the “C-side” 1+Nub chemistry.

Scaffold Permutation

The above examples involved making a unique or unusual building block that could be used as an intermediate for monophore synthesis. The following examples illustrate chemistries that lead to a unique variant of the chemotype.

Preparation of Tertiary Amines.

A solid-phase synthesis route was adapted for the preparation of tertiary amines. Briefly, immobilization of the cysteamine linker to BAL resin provides a common intermediate for a number of different syntheses. In the present example, the resin-bound tether linker is acylated with an amino acid, the amino acid is then deprotected and then alkylated with an appropriate aldehyde to prepare the desired tertiary amine. Arylation is also possible using established methods. The procedure for tertiary amine synthesis is shown schematically, below:

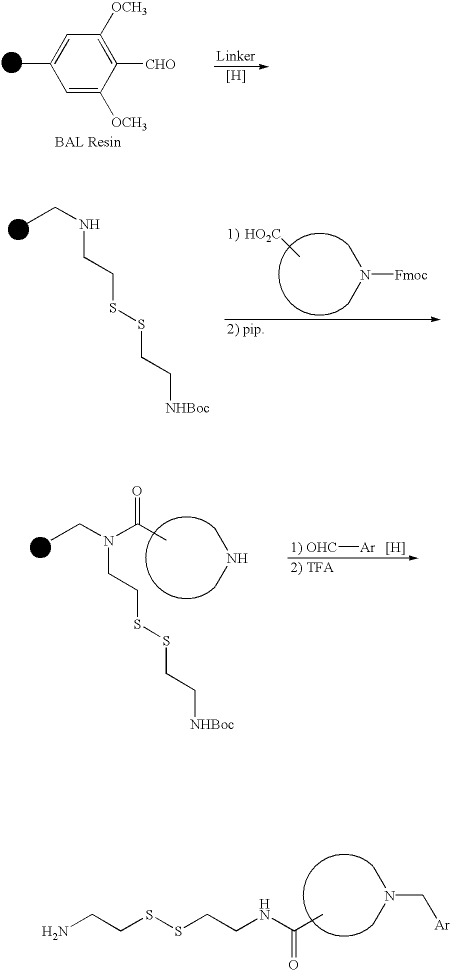

Preparation of Aminothiazoles.

Aminothiazoles are being prepared, and their synthesis utilizes the same resin-bound linker intermediate employed for the tertiary amine synthesis. Approximately 400 of these compounds have been prepared and are being purified by HPLC prior to release into the monophore collection.

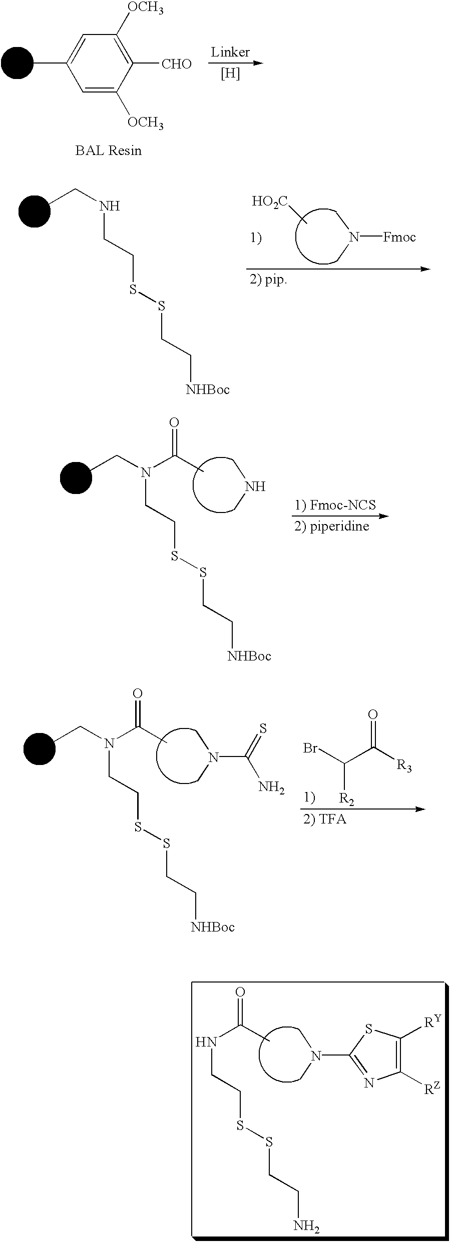

D. Exemplary Library Syntheses:

### EXAMPLE 1

Library 000004 consists of 484 peptidomimetic compounds connected to the cystamine-derived tethering linker. This library consists of four conformationally constrained amino acid “scaffolds” that were acylated with 121 different carboxylic acids. General formula for the library is as follows: 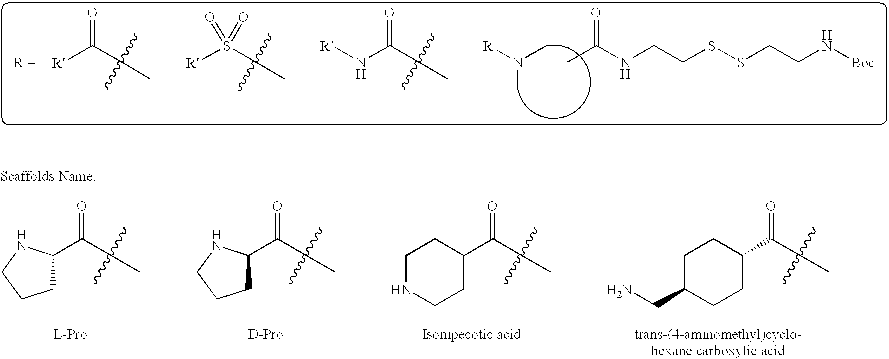
where R′ is defined as for R⁵ and R⁶, as described generally herein.

### EXAMPLE 2

Library 000005 consists of 453 peptidomimetic compounds connected to the cystamine-derived tethering linker. This library consists of four conformationally-constrained amino acid “scaffolds” that were acylated with 121 different carboxylic acids. General formula for the library is as follows: 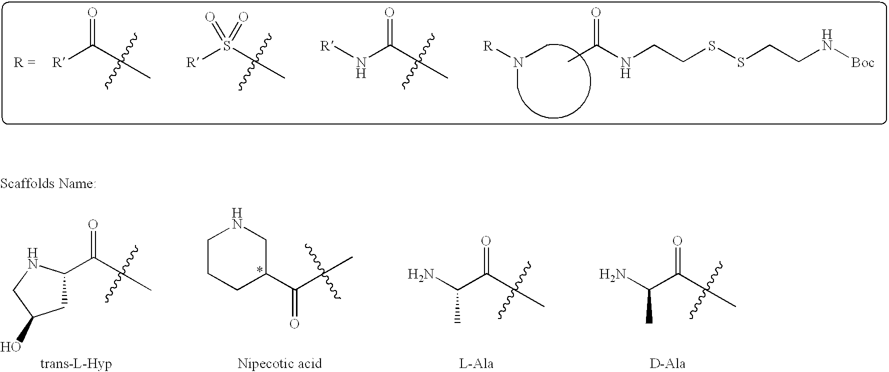
where R′ is defined as for R⁵ and R⁶, as described generally herein.

### EXAMPLE 3

Library 000006 consists of 453 peptidomimetic compounds connected to the cystamine-derived tethering linker. This library consists of four conformationally-constrained amino acid “scaffolds” that were acylated with 121 different carboxylic acids. General formula for the library is as follows: 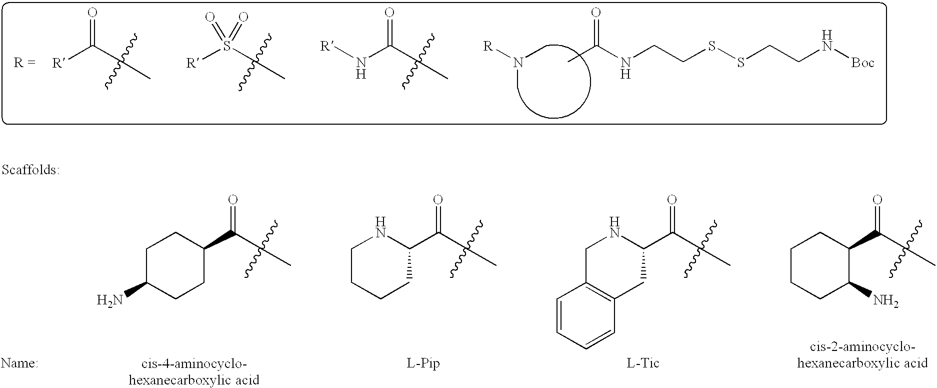
where R′ is defined as for R⁵ and R⁶, as described generally herein.

### EXAMPLE 4

Library 000007 consists of 681 peptidomimetic compounds connected to the cystamine-derived tethering linker. This library consists of six conformationally-constrained amino acid “scaffolds” that were acylated with 121 different carboxylic acids. General formula for the library is as follows: 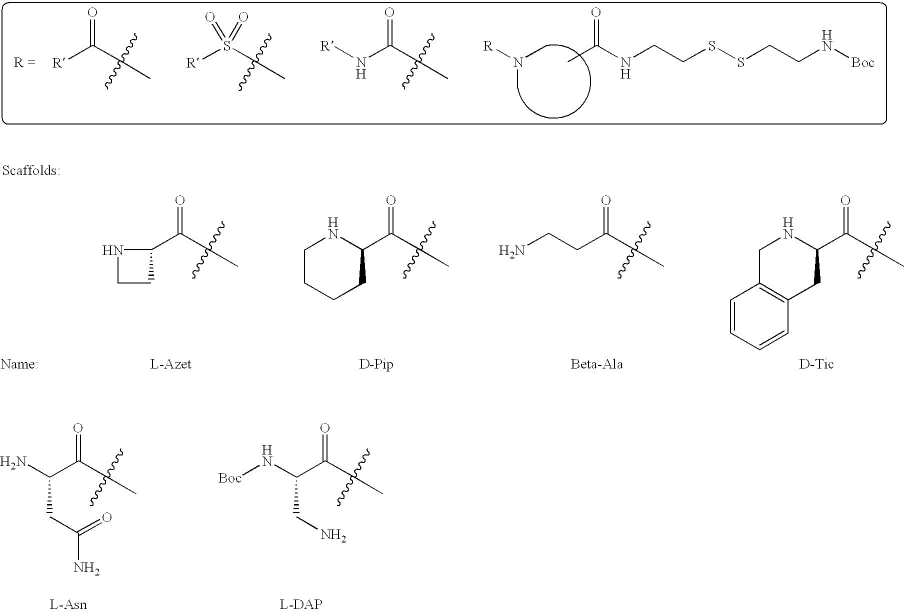
where R′ is defined as for R⁵ and R⁶, as described generally herein.

### EXAMPLE 5

Library 000014 was prepared from four conformationally-constrained amino acid “scaffolds” that were used to acylated 293 diverse primary and secondary amines (1172 reactions). After eliminating compounds that failed QC, 690 compounds were released.

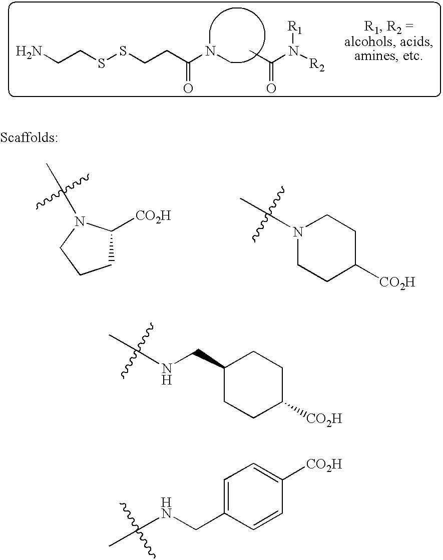

### EXAMPLE 6

Library 000017 was prepared from 10 conformationally-constrained amino acid “scaffolds” that were used to acylate 220 diverse primary and secondary amines (approx. 2200 reactions). After eliminating compounds that failed QC, 833 compounds were released. General formula for the library is as follows:

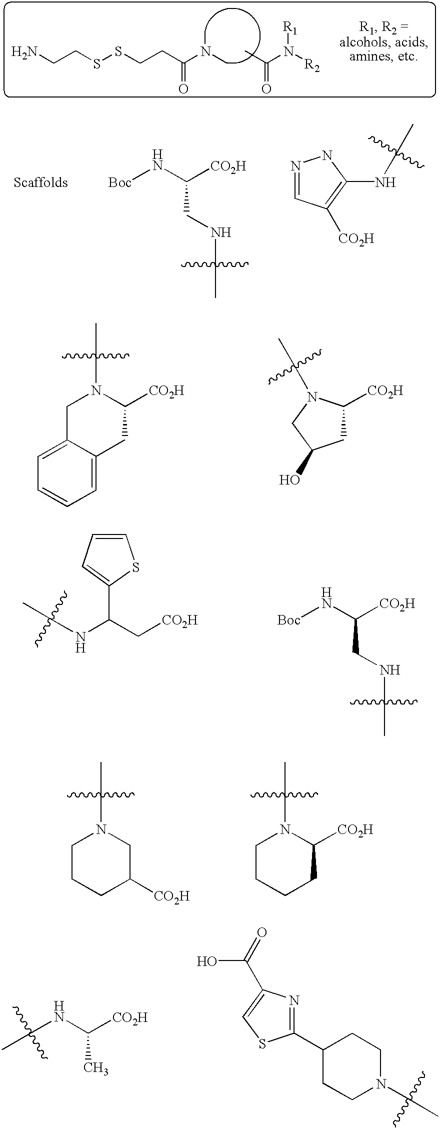

### EXAMPLE 7

Library 000018 was prepared from 9 conformationally-constrained amino acid “scaffolds” that were used to acylate 220 diverse primary and secondary amines (approx. 2000 reactions). After eliminating compounds that failed QC, 811 compounds were released. General formula for the library is as follows:

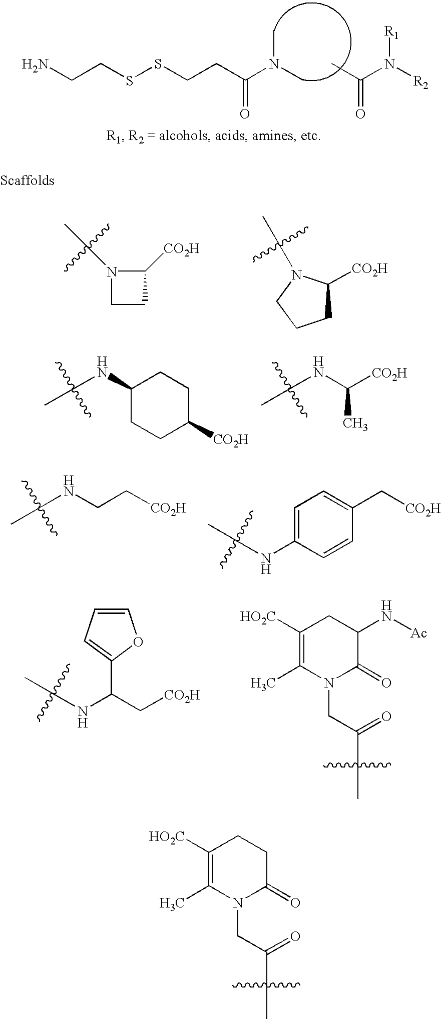

### EXAMPLE 8

Library 000016 was prepared from five thiazole core scaffolds, that were used to acylated 220 diverse primary and secondary amines (1100 reactions). 750 of these passed QC and were added to the screening collection.

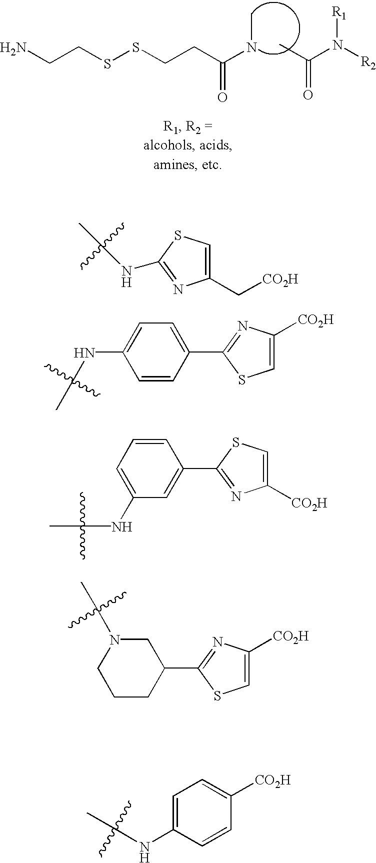

E. Identification:

Following tethering the ligand to a TBM, the ligands bound to a target can be readily detected and identified by mass spectroscopy (MS). MS detects molecules based on mass-to-charge ratio (m/z) and thus can resolve molecules based on their sizes (reviewed in Yates, Trends Genet. 16: 5-8 [2000]). A mass spectrometer first converts molecules into gas-phase ions, then individual ions are separated on the basis of m/z ratios and are finally detected. A mass analyzer, which is an integral part of a mass spectrometer, uses a physical property (e.g. electric or magnetic fields, or time-of-flight [TOF]) to separate ions of a particular m/z value that then strikes the ion detector.

Mass spectrometers are capable of generating data quickly and thus have a great potential for high-throughput analysis. MS offers a very versatile tool that can be used for drug discovery. Mass spectroscopy may be employed either alone or in combination with other means for detection or identifying the organic compound ligand bound to the target. Techniques employing mass spectroscopy are well known in the art and have been employed for a variety of applications (see, e.g., Fitzgerald and Siuzdak, Chemistry & Biology 3: 707-715 [1996]; Chu et al., J. Am. Chem. Soc. 118: 7827-7835 [1996]; Siudzak, Proc. Natl. Acad. Sci. USA 91: 11290-11297 [1994]; Burlingame et al., Anal. Chem. 68: 599R-651R [1996]; Wu et al., Chemistry & Biology 4: 653-657 [1997]; and Loo et al., Am. Reports Med. Chem. 31: 319-325 [1996]).

Other techniques that may find use for identifying the organic compound bound to the target molecule include, for example, nuclear magnetic resonance (NMR), capillary electrophoresis, X-ray crystallography, and the like, all of which will be well known to those skilled in the art.

1. A compound having the structure (I): 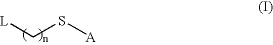
wherein A is —S(CH₂)ₚR^{A1} or —S(O)₂R^{A2}, wherein p is 1-5, R^{A1} is —NR^{A3}R^{A4}; OR^{A3}; SR^{A3}; —NHCOR^{A3}; —NHCONR^{A3}R^{A4}; —NR^{A3}R^{A4}R⁵⁺X⁻, wherein X is a halogen; —COOR^{A3}; CONR^{A3}R^{A4}; —SO₃R^{A3}; —OPO₃R^{A3}; —SO₂R^{A3}; and wherein R^{A2} is an aliphatic, heteroaliphatic, aryl, or heteroaryl moiety, and each occurrence of R^{A3}, R^{A4}, and R^{A5} is independently hydrogen, a protecting group, or an aliphatic, heteroaliphatic, aryl or heteroaryl moiety;
n is 0-5;
L is a moiety having one of the structures: 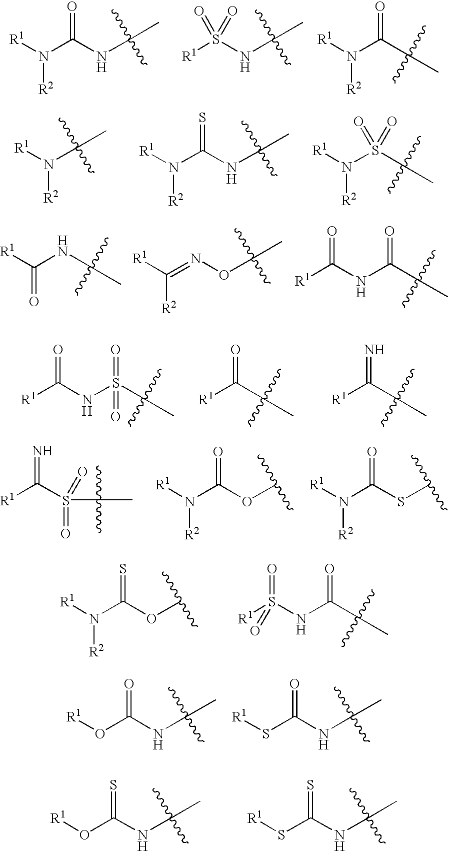
each occurrence of R¹ and R² is independently hydrogen, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, or wherein R¹ and R² taken together are a cycloaliphatic, heterocycloaliphatic, aryl or heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

2. The compound of claim 1, wherein L is one of the following structures:

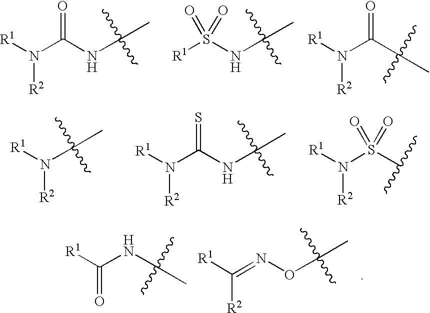

3. The compound of claim 1, wherein 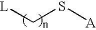
represents one of the structures: 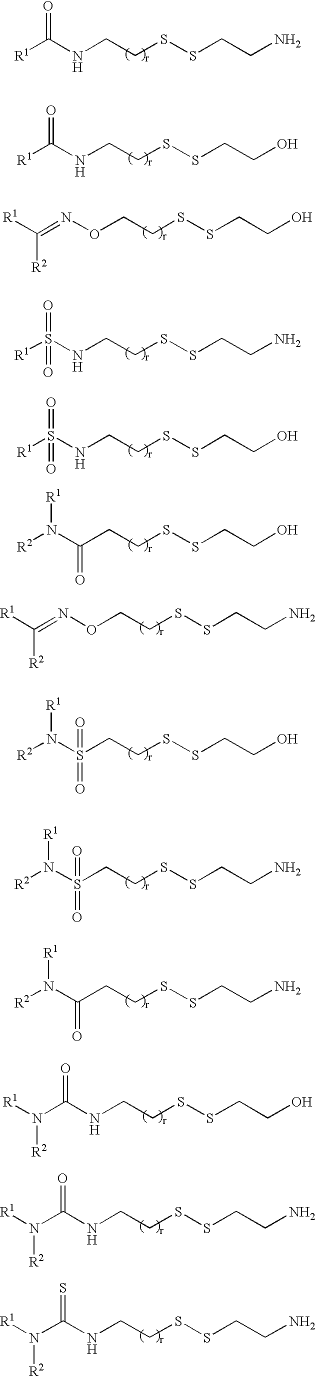 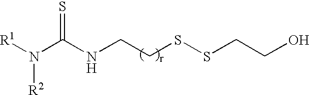
wherein r is 1 or 2; t is 0, 1 or 2; and R^{A2} is an alkyl, heteroalkyl, aryl, heteroaryl, -(alkyl)aryl, -(alkyl)heteroaryl, -(heteroalkyl)aryl, or -(heteroalkyl)heteroaryl moiety.

4. The compound of claim 1, wherein 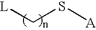
represents one of the structures: 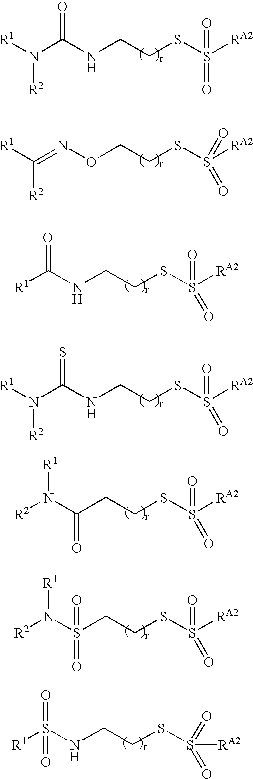
wherein r is 1 or 2; R^{A2} is an alkyl, heteroalkyl, aryl, heteroaryl, -(alkyl)aryl, -(alkyl)heteroaryl, -(heteroalkyl)aryl, or -(heteroalkyl)heteroaryl moiety.

5. The compound of claim 4, whereinR^{A2} is methyl or phenyl.

6. The compound of claim 1, wherein one or both of R¹ or R² is 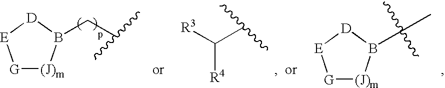
wherein R¹ and R² taken together form a cyclic moiety having the structure:
wherein B—D, D—E, E—G, G—J, two or more occurrences of J, and J—B are each independently joined by a single or double bond as valency and stability permit, wherein B is N, CH or C, D is —NR^{D}—, ═N—, —O—, —CHR^{D}—, or ═CR^{D}—, E is —NR^{E}—, ═N—, —O—, —CHR^{E}—, or ═CR^{E}—, G is —NR^{G}—, ═N—, —O—, —CHR^{G}—, or ═CR^{G}—, each occurrence of J is independently —NR^{J}—, ═N—, —O—, —CHR^{J}—, or ═CR^{J}—,
m is 0-4 and p is 0-4,
each occurrence of R³, R⁴, R^{D}, R^{E}, R^{G} and R^{J} is independently hydrogen, a protecting group, —(CR⁷R⁸)_{q}NR⁵R⁶, —(CR⁷R⁸)_{q}OR⁵, —(CR⁷R⁸)_{q}SR⁵, —(CR⁷R⁵)_{q}(C═O)R⁵, —(CR⁷R⁸)_{q}(C═O)OR⁵; —(CR⁷R⁸)_{q}(C═O)NR⁵R⁶, —(CR⁷R⁸)_{q}S(O)₂R⁵, —(CR⁷R⁸)_{q}NR⁵(C═O)R⁶, —(CR⁷R⁸)_{q}NR⁵(C═O)OR⁶, —(CR⁷R⁸)_{q}S(O)₂NR⁵R⁶, —(CR⁷R⁸)_{q}NR⁵S(O)₂R⁶, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
q is 0-4; and
each occurrence of R⁵, R⁶, R⁷ and R⁸ is independently hydrogen, a protecting group, or an aliphatic, heteroalipahtic, aryl, heteroaryl, -(aliphatic)aryl, -(aliphatic)heteroaryl, -(heteroalipahtic)aryl, or -(heteroaliphatic)heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

7. The compound of claim 1, wherein L is 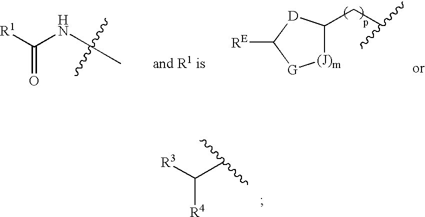
wherein m is 0-4, p is 0-4, D is CHR^{D} or NR^{D}, G is CHR^{G} or NR^{G}, and each occurrence of J is independently CHR^{J} or NR^{J}, wherein each occurrence of R^{D}, R^{E}, R^{G}, R^{J}, R³, and R⁴ is independently hydrogen, a protecting group, —(CR⁷R⁸)_{q}NR⁵R⁶, —(CR⁷R⁸)_{q}OR⁵, —(CR⁷R⁸)_{q}SR⁵, —(CR⁷R⁸)_{q}(C═O)R⁵, —(CR⁷R⁸)_{q}(C═O)NR⁵R⁶, —(CR⁷R⁸)_{q}S(O)₂R⁵, —(CR⁷R⁸)_{q}NR⁵(C═O)R⁶, —(CR⁷R⁸)_{q}S(O)₂NR⁵R⁶, —(CR⁷R⁸)_{q}NR⁵S(O)₂R⁶, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, wherein q is 0-4; and wherein each occurrence of R⁵ and R⁶ is independently hydrogen, a protecting group, or an aliphatic, heteroalipahtic, aryl, heteroaryl, -(aliphatic)aryl, -(aliphatic)heteroaryl, -(heteroalipahtic)aryl, or -(heteroaliphatic)heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

8. The compound of claim 1, wherein L is 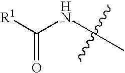
and R¹ is one of the structures:

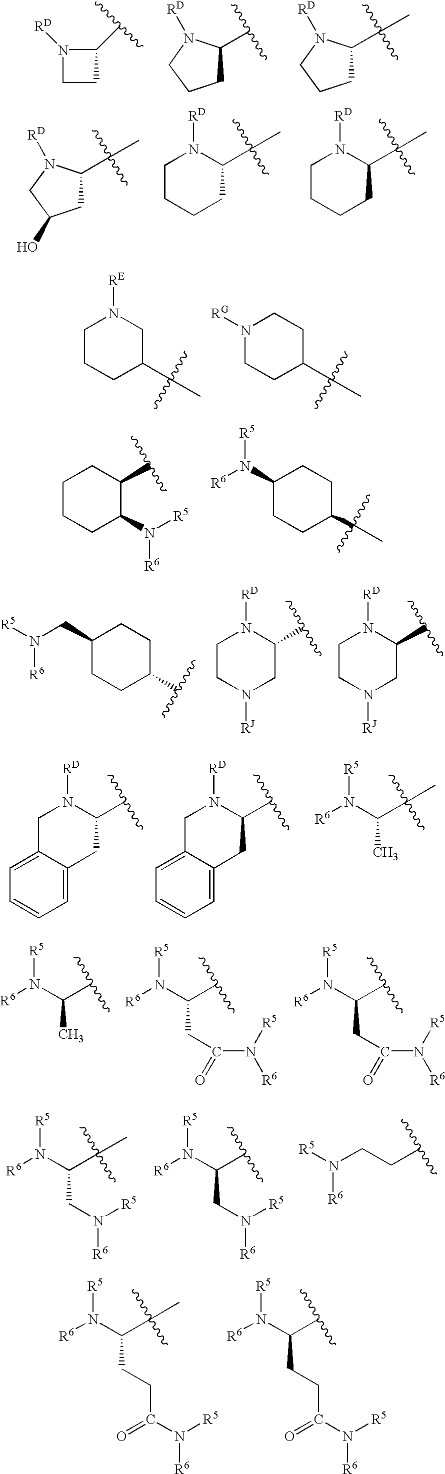

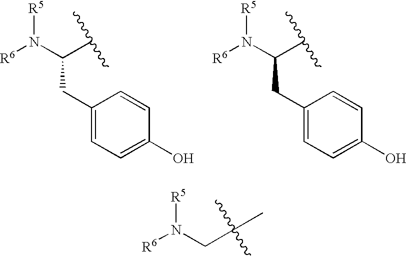

9. The compound of claim 1, wherein L is 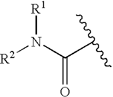
and one or both of R¹ and R² is 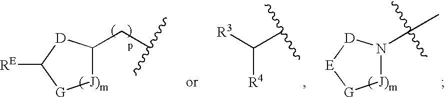
or wherein R¹ and R² taken together with N form a cyclic structure:
wherein B—D, D—E, E—G, G—J, two or more occurrences of J, and J—B are each independently joined by a single or double bond as valency and stability permit, wherein B is N, CH or C, D is —NR^{D}—, ═N—, —O—, —CHR^{D}—, or ═CR^{D}—, E is —NR^{E}—, ═N—, —O—, —CHR^{E}—, or ═CR^{E}—, G is —NR^{G}—, ═N—, —O—, —CHR^{G}—, or ═CR^{G}—, each occurrence of J is independently —NR^{J}—, ═N—, —O—, —CHR^{J}—, or ═CR^{J}—,
m is 0-4and p is 0-4,
each occurrence of R³, R⁴, R^{D}, R^{E}, R^{G} and R^{J} is independently hydrogen, a protecting group, —(CR⁷R⁸)_{q}NR⁵R⁶, —(CR⁷R⁸)_{q}OR⁵, —(CR⁷R⁸)_{q}SR⁵, —(CR⁷R⁵)_{q}(C═O)R⁵, —(CR⁷R⁸)_{q}(C═O)OR⁵; —(CR⁷R⁸)_{q}(C═O)NR⁵R⁶, —(CR⁷R⁸)_{q}S(O)₂R⁵, —(CR⁷R⁸)_{q}NR⁵(C═O)R⁶, —(CR⁷R⁸)_{q}NR⁵(C═O)OR⁶, —(CR⁷R⁸)_{q}S(O)₂NR⁵R⁶, —(CR⁷R⁸)_{q}NR⁵S(O)₂R⁶, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
q is 0-4; and
each occurrence of R⁵, R⁶, R⁷ and R⁸ is independently hydrogen, a protecting group, or an C aliphatic, heteroalipahtic, aryl, heteroaryl, -(aliphatic)aryl, -(aliphatic)heteroaryl, -(heteroalipahtic)aryl, or -(heteroaliphatic)heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

10. The compound of claim 1, wherein L is 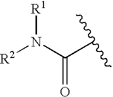
and one or both of R¹ and R² is a moiety having one of the following structures, or wherein R¹ and R² taken together with N form a cyclic moiety having one of the following structures:

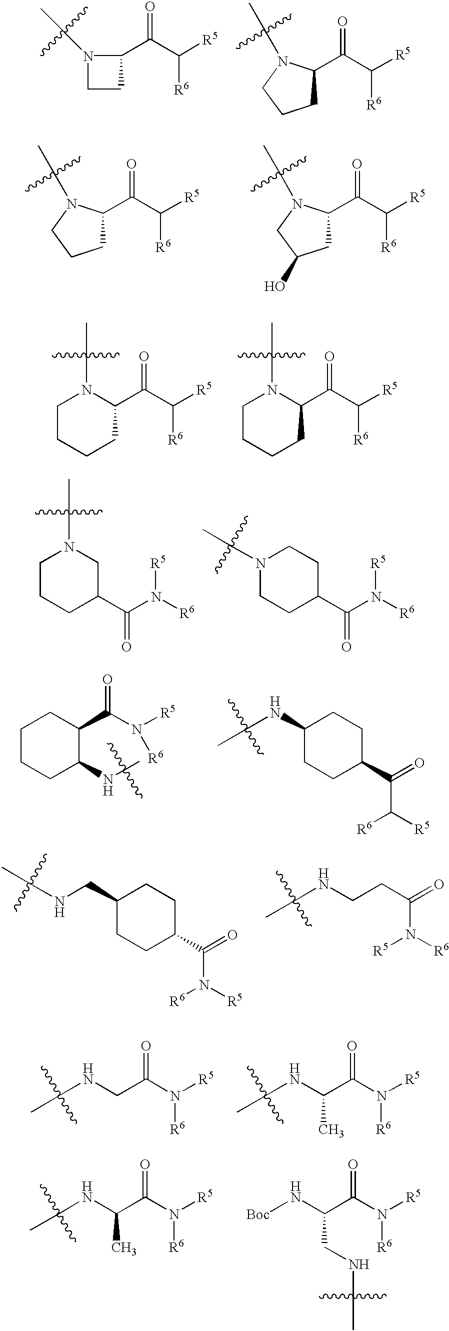

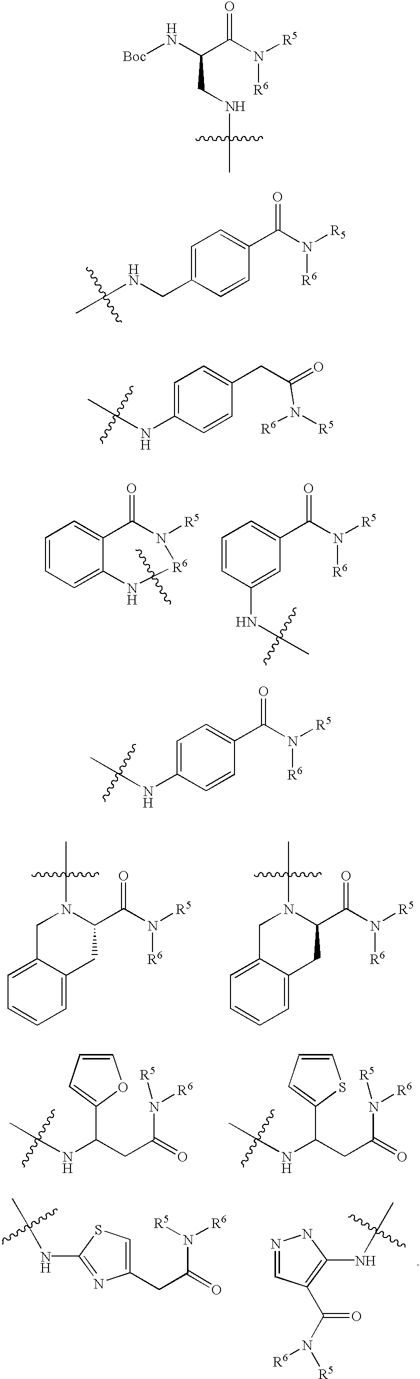

11. The compound of claim 1, wherein L is 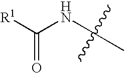
R¹ has one of the following structures:

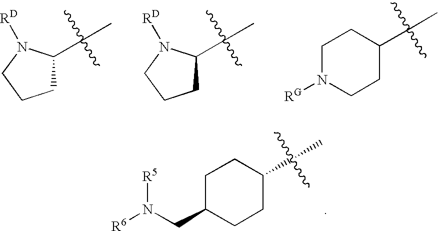

12. The compound of claim 1, wherein L is 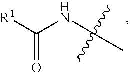
and R¹ has one of the following structures:

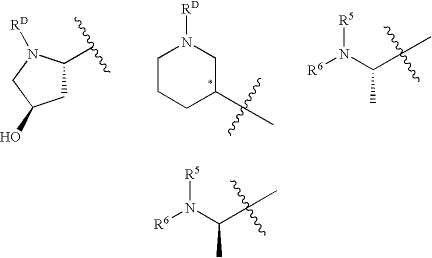

13. The compound of claim 1, wherein L is 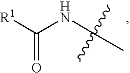
and R¹ has one of the following structures:

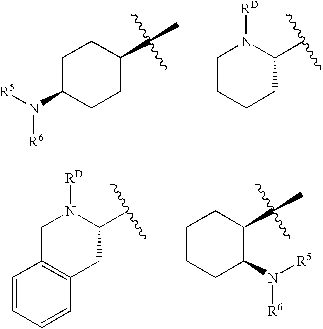

14. The compound of claim 1, wherein L is 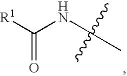
and R¹ has one of the following structures:

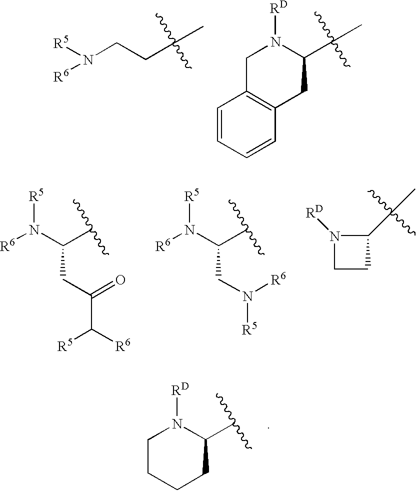

15. The compound of claim 1, wherein L is 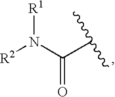
and R¹ has one of the following structures:

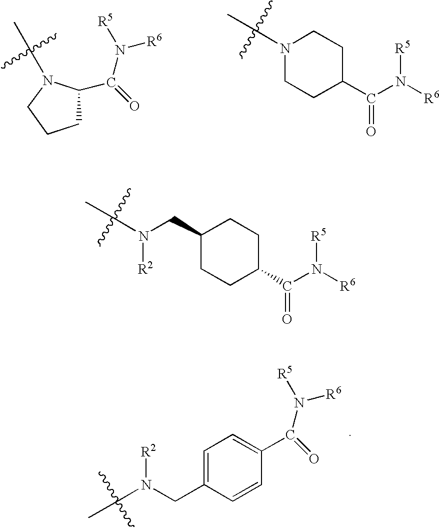

16. The compound of claim 1, wherein L is 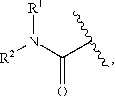
and R¹ is one of the following structures:

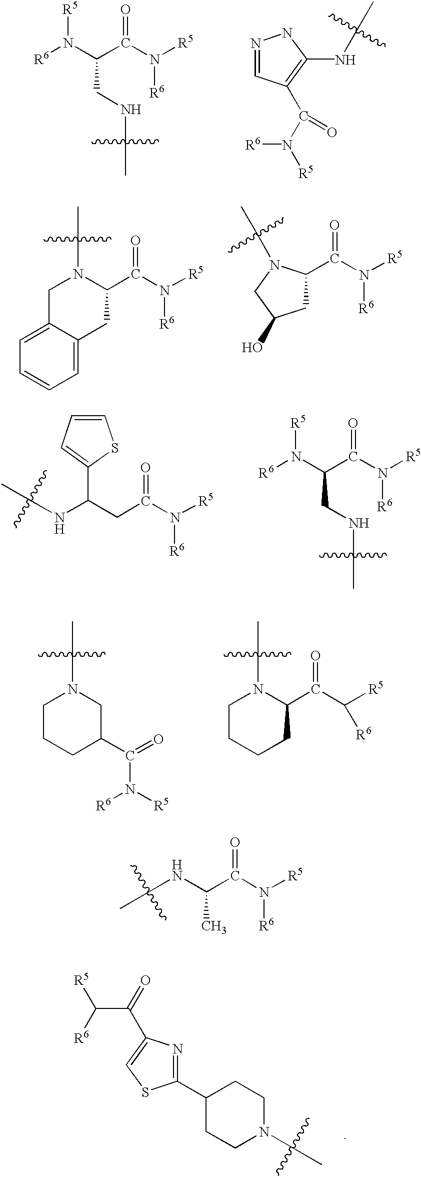

17. The compound of claim 1, wherein L is 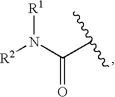
and R¹ is one of the following structures:

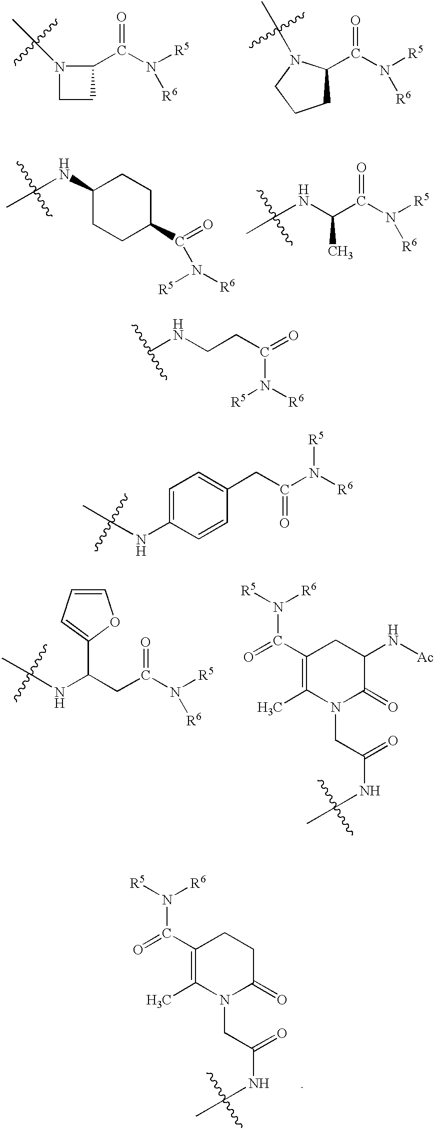

18. The compound of claim 1, wherein L is 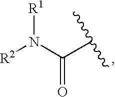
and R¹ and R² are each independently hydrogen or a cycloaliphatic, heterocycloaliphatic, aryl or heteroaryl moiety optionally substituted with a substituted heteroaryl moiety.

19. The compound of claim 18, wherein the substituted heteroaryl moiety has one of the structures: 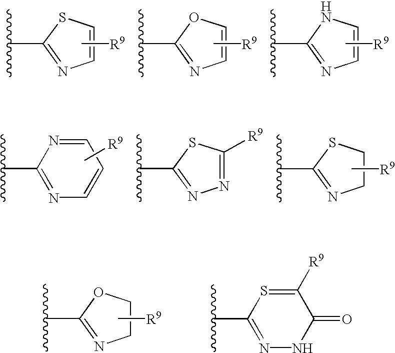
wherein R⁹ is —COO(R¹⁰), —CO(R¹⁰), —CO(NR¹⁰R¹¹), —NR¹⁰OR¹⁰, —NR¹⁰OCOR¹¹, —OR¹⁰, or —SR¹⁰, wherein each occurrence of R¹⁰ is independently hydrogen, a protecting group, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

20. The compound of claim 19, wherein R¹ and R² represent one of the following structures: 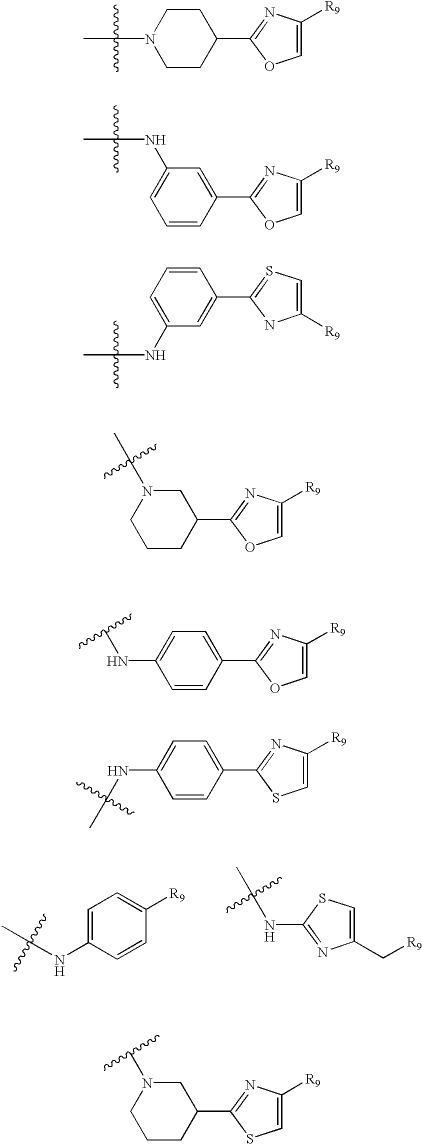
wherein R⁹ is COOH or is CO(NR¹⁰OR¹¹), wherein each occurrence of R¹⁰ and R¹¹ is independently hydrogen, a protecting group, or an aliphatic, heteroaliphatic, aryl, heteroaryl, -(aliphatic)aryl, -(aliphatic)heteroaryl, -(heteroaliphatic)aryl, or -(heteroaliphatic)heteroaryl,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

21. A library of compounds comprising a plurality of compounds having the structure (I): 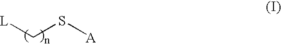
wherein A is —S(CH₂)ₚR^{A1} or —S(O)₂R^{A2}, wherein p is 1-5, R^{A1} is —NR^{A3}R^{A4}; OR^{A3}; SR^{A3}; —NHCOR^{A3}; —NHCONR^{A3}R^{A4}; —NR^{A3}R^{A4}R^{A5+}X⁻, wherein X is a halogen; —COOR^{A3}; CONR^{A3}R^{A4}; —SO₃R^{A3}; —OPO₃R^{A3}; —SO₂R^{A3}; and wherein R^{A2} is an aliphatic, heteroaliphatic, aryl, or heteroaryl moiety, and each occurrence of R^{A3}, R^{A4}, and R^{A5} is independently hydrogen, a protecting group, or an aliphatic, heteroaliphatic, aryl or heteroaryl moiety;
n is 0-5;
L is a moiety having one of the structures: 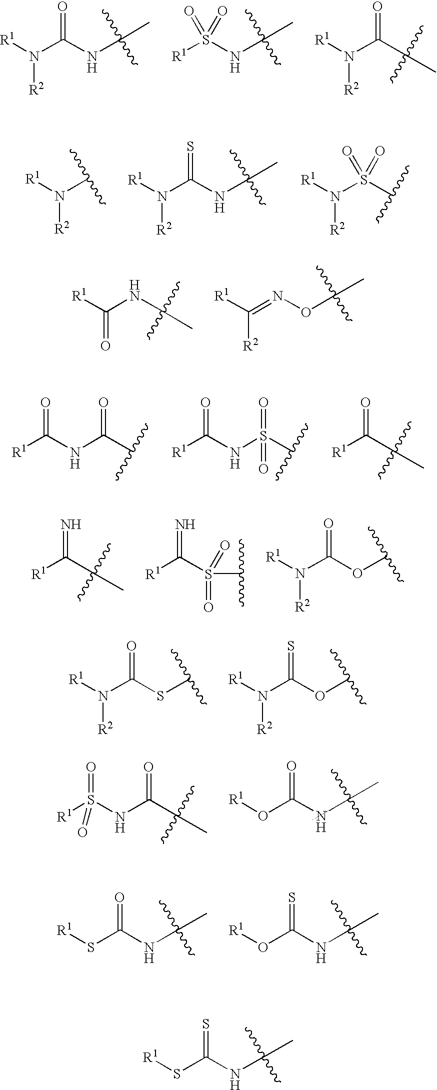
each occurrence of R¹ and R² is independently hydrogen, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, or wherein R¹ and R² taken together are a cycloaliphatic, heterocycloaliphatic, aryl or heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

22. The library of claim 21, wherein L is one of the following structures:

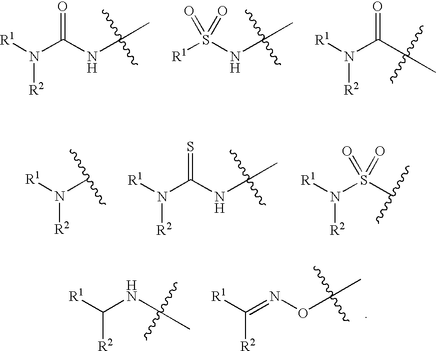

23. The library of claim 21, wherein 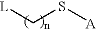
represents one of the structures: 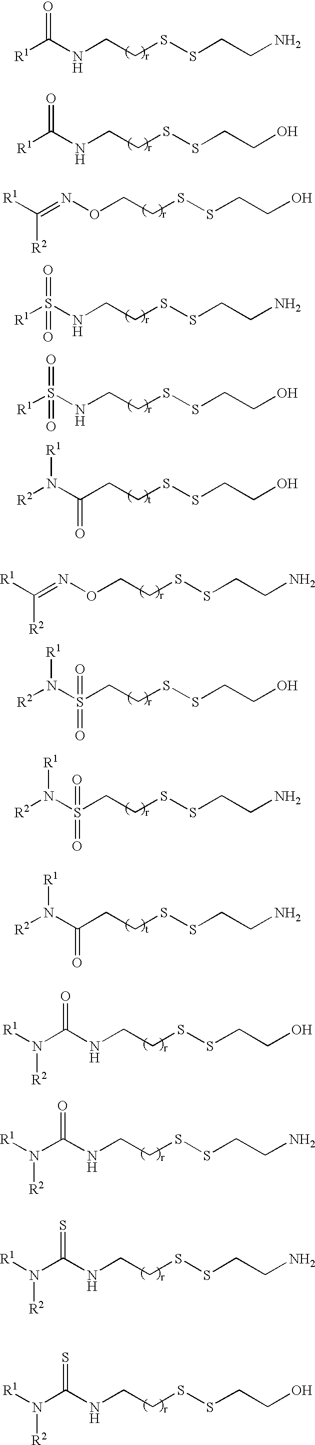
wherein r is 1 or 2; t is 0, 1 or 2; and RA is an alkyl, heteroalkyl, aryl, heteroaryl, -(alkyl)aryl, -(alkyl)heteroaryl, -(heteroalkyl)aryl, or -(heteroalkyl)heteroaryl moiety.

24. The library of claim 21, wherein 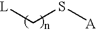
represents one of the structures: 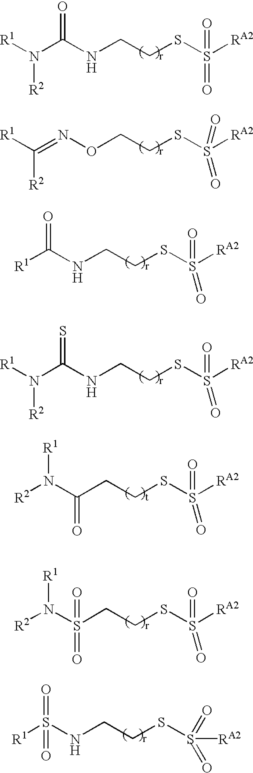
wherein r is 1 or 2; and R^{A2} is an alkyl, heteroalkyl, aryl, heteroaryl, -(alkyl)aryl, -(alkyl)heteroaryl, -(heteroalkyl)aryl, or -(heteroalkyl)heteroaryl moiety.

25. The library of claim 24, wherein R^{A2} is methyl or phenyl.

26. The library of claim 21, wherein one or both of R¹ or R² is 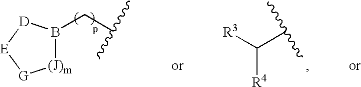
wherein R¹ and R² taken together form a cyclic moiety having the structure: 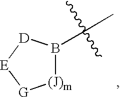
wherein B—D, D—E, E—G, G—J, two or more occurrences of J, and J—B are each independently joined by a single or double bond as valency and stability permit, wherein B is N, CH or C, D is —NR^{D}—, ═N—, —O—, —CHR^{D}—, or ═CR^{D}, E is —NR^{E}—, ═N—, —O—, —CHR^{E}—, or ═CR^{E}—, G is —NR^{G}—, ═N—, —O—, —CHR^{G}—, or ═CR^{G}—, each occurrence of J is independently —NR^{J}—, ═N—, —O—, —CHR^{J}—, or ═CR^{J}—,
m is 0-4and p is 0-4,
each occurrence of R³, R⁴, R^{D}, R^{E}, R^{G} and R^{J} is independently hydrogen, a protecting group, —(CR⁷R⁸)_{q}NR⁵R⁶, —(CR⁷R⁸)_{q}OR⁵, —(CR⁷R⁸))_{q}SR⁵, —(CR⁷R⁸)_{q}(C═O)R⁵, —(CR⁷R⁸)_{q}(C═O)OR⁵; —(CR⁷R⁸)_{q}(C═O)NR⁵R⁶, —(CR⁷R⁸)_{q}S(O)₂R⁵, —(CR⁷R⁸)_{q}NR⁵(C═O)R⁶, —(CR⁷R⁸)_{q}NR⁵(C═O)OR⁶, —(CR⁷R⁸)_{q}S(O)₂NR⁵R⁶, —(CR⁷R⁸)_{q}NR⁵S(O)₂R⁶, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
q is 0-4; and
each occurrence of R⁵, R⁶, R⁷ and R⁸ is independently hydrogen, a protecting group, or an aliphatic, heteroalipahtic, aryl, heteroaryl, -(aliphatic)aryl, -(aliphatic)heteroaryl, -(heteroalipahtic)aryl, or -(heteroaliphatic)heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

27. The library of claim 21, wherein L is 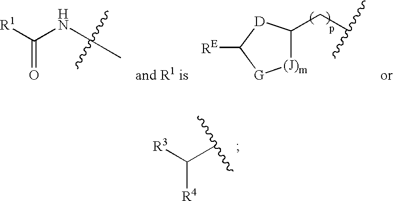
wherein m is 0-4, p is 0-4, D is CHR^{D} or NR^{D}, G is CHR^{G} or NR^{G}, and each occurrence of J is independently CHR^{J} or NR^{J}, wherein each occurrence of R^{D}, R^{E}, R^{G}, R^{J}, R³, and R⁴ is independently hydrogen, a protecting group, —(CR⁷R⁸)_{q}NR⁵R⁶, —(CR⁷R⁸)_{q}OR⁵, —(CR⁷R⁸)_{q}SR⁵, —(CR⁷R⁸)_{q}(C═O)R⁵, —(CR⁷R⁸)_{q}(C═O)NR⁵R⁶, —(CR⁷R⁸)_{q}S(O)₂R⁵, —(CR⁷R⁸)_{q}NR⁵(C═O)R⁶, —(CR⁷R⁸)_{q}S(O)₂NR⁵R⁶, —(CR⁷R⁸)_{q}NR⁵S(O)₂R⁶, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety, wherein q is 0-4; and wherein each occurrence of R⁵ and R⁶ is independently hydrogen, a protecting group, or an aliphatic, heteroalipahtic, aryl, heteroaryl, -(aliphatic)aryl, -(aliphatic)heteroaryl, -(heteroalipahtic)aryl, or -(heteroaliphatic)heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

28. The library of claim 21, wherein L is 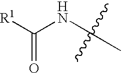
and R¹ is one of the structures:

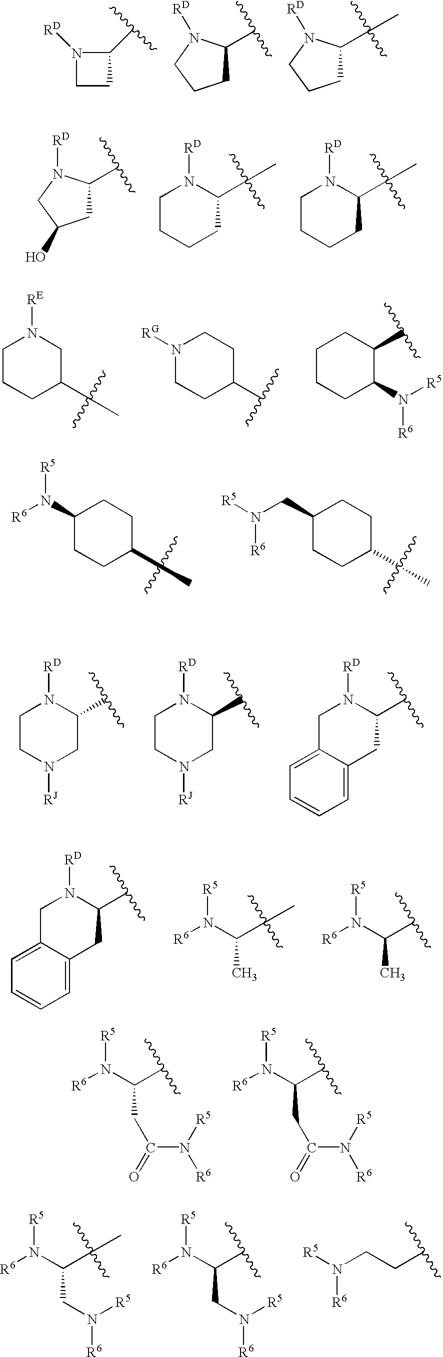

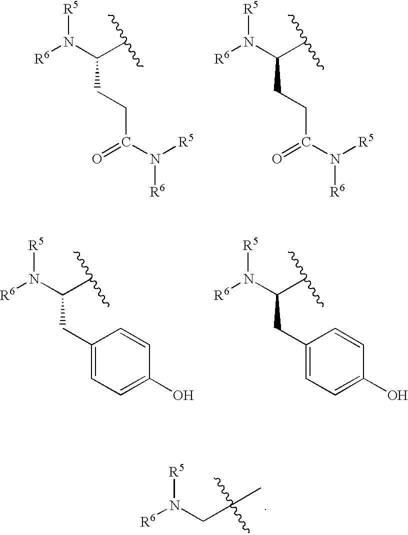

29. The library of claim 21, wherein L is 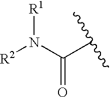
and one or both of R¹ and R² is 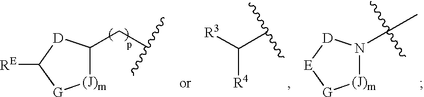
or wherein R¹ and R² taken together with N form a cyclic structure:
wherein B—D, D—E, E—G, G—J, two or more occurrences of J. and J—B are each independently joined by a single or double bond as valency and stability permit, wherein B is N. CH or C, D is —NR^{D}—, ═N—, —O—, —CHR^{D}—, or ═CR^{D}—, E is —NR^{E}—, ═N—, —O—, —CHR^{E}—, or ═CR^{E}—, G is —NR^{G}—, ═N—, —O—, —CHR^{G}—, or ═CR^{G}—, each occurrence of J is independently —NR^{J}—, ═N—, —O—, —CHR^{J}—, or ═CR^{J}—,
m is 0-4and p is 0-4,
each occurrence of R³, R⁴, R^{D}, R^{E}, R^{G} and R^{J} is independently hydrogen, a protecting group, —(CR⁷R⁸)_{q}NR⁵R⁶, —(CR⁷R⁸)_{q}OR⁵, —(CR⁷R⁸)_{q}SR⁵, —(CR⁷R⁸)_{q}(C═O)R⁵, —(CR⁷R⁸)_{q}(C═O)OR⁵; —(CR⁷R⁸)_{q}(C═O)NR⁵R⁶, —(CR⁷R⁸)_{q}S(O)₂R⁵, —(CR⁷R⁸)_{q}NR⁵(C═O)R⁶, —(CR⁷R⁸)_{q}NR⁵(C═O)OR⁶, —(CR⁷R⁸)_{q}S(O)₂NR⁵R⁶, —(CR⁷R⁸)_{q}NR⁵S(O)₂R⁶, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
q is 0-4; and
each occurrence of R⁵, R⁶, R⁷ and R⁸ is independently hydrogen, a protecting group, or an aliphatic, heteroalipahtic, aryl, heteroaryl, -(aliphatic)aryl, -(aliphatic)heteroaryl, -(heteroalipahtic)aryl, or -(heteroaliphatic)heteroaryl moiety;
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

30. The library of claim 21, wherein L is 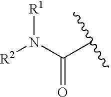
and one or both of R¹ and R² is a moiety having one of the following structures, or wherein R¹ and R² taken together with N form a cyclic moiety having one of the following structures:

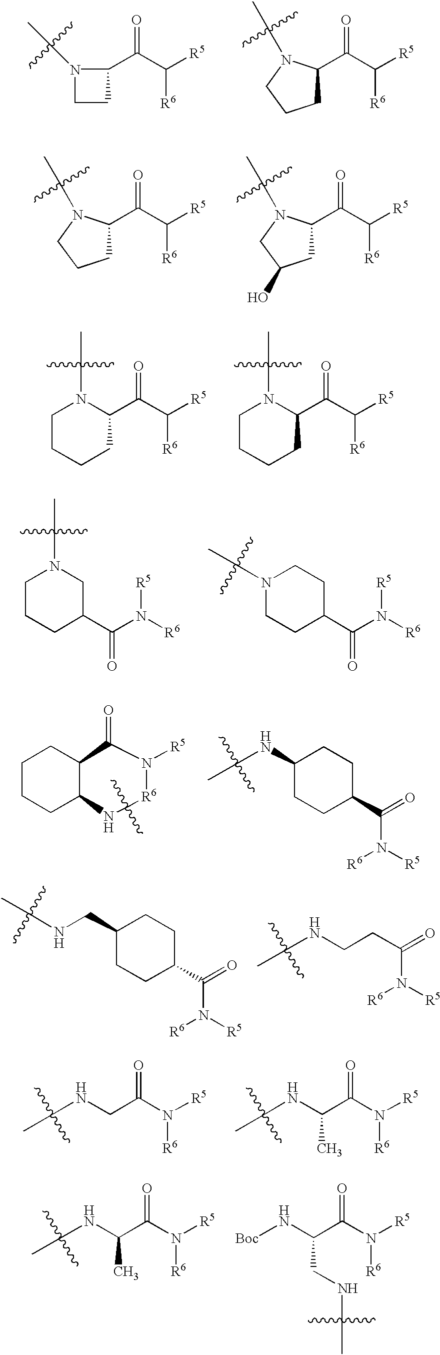

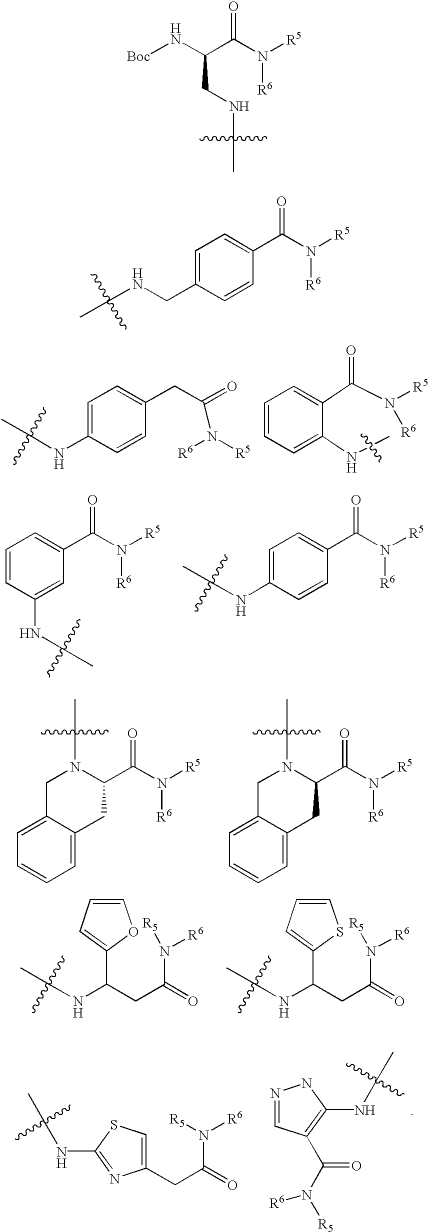

31. The library of claim 21, wherein L is 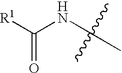
R¹ has one of the following structures:

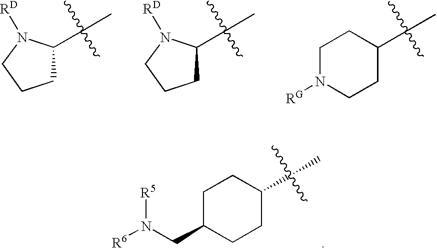

32. The library of claim 21, wherein L is 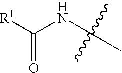
and R¹ has one of the following structures:

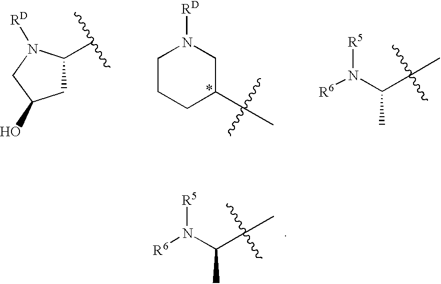

33. The library of claim 21, wherein L is 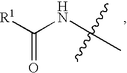
and R¹ has one of the following structures:

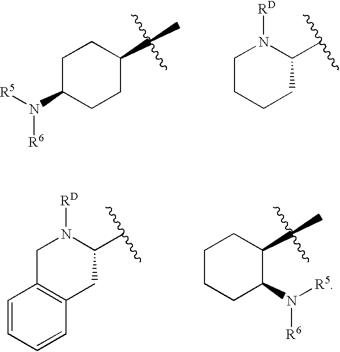

34. The library of claim 21, wherein L is 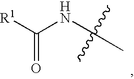
and R¹ has one of the following structures:

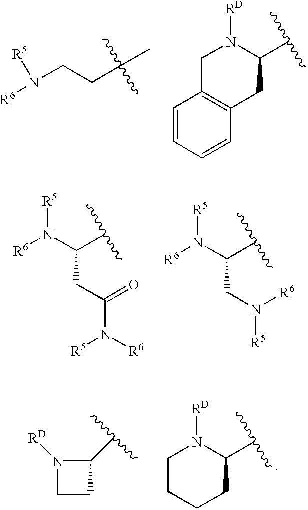

35. The library of claim 21, wherein L is 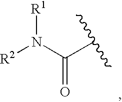
and R¹ has one of the following structures:

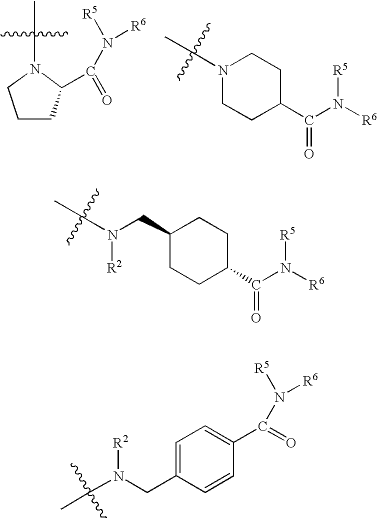

36. The library of claim 21, wherein L is 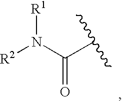
and R¹ has one of the following structures:

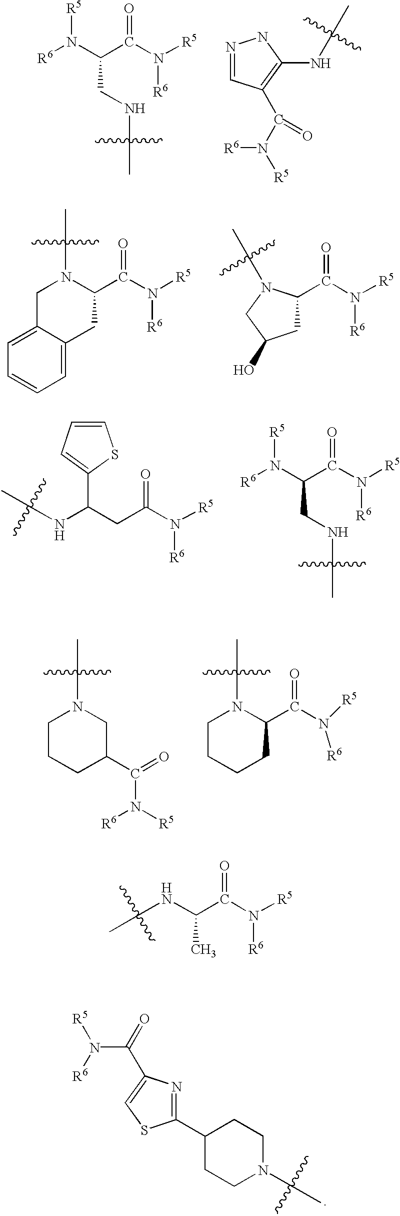

37. The library of claim 21, wherein L is 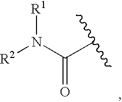
and R¹ is one of the following structures:

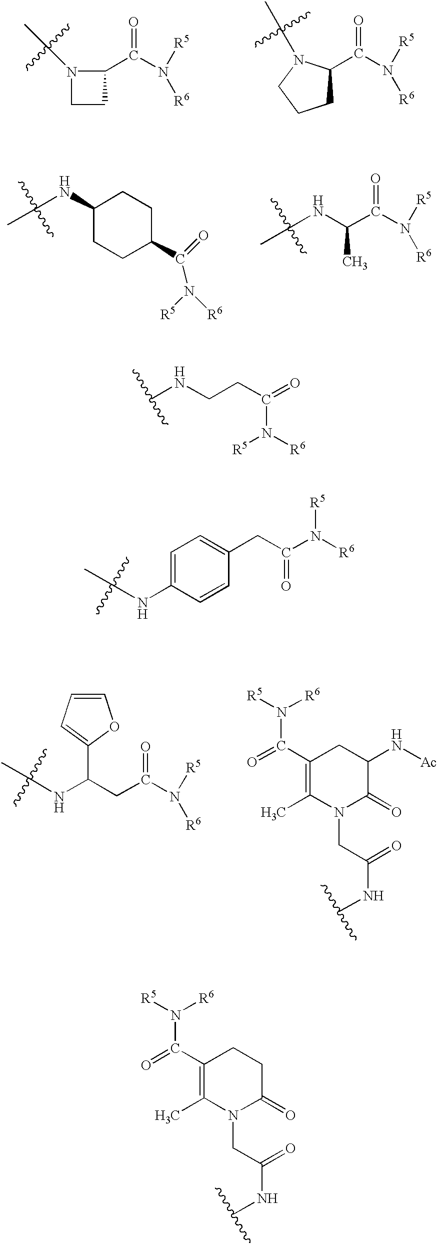

38. The library of claim 21, wherein L is 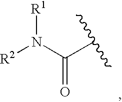
and R¹ and R² are each independently hydrogen or a cycloaliphatic, heterocycloaliphatic, aryl or heteroaryl moiety optionally substituted with a substituted heteroaryl moiety.

39. The compound of claim 38, wherein the substituted heteroaryl moiety has one of the structures: 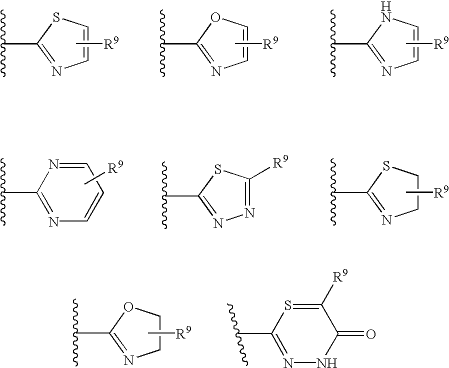
wherein R⁹ is —COO(R¹⁰), —CO(R¹⁰), —CO(NR¹⁰OR¹¹), —NR¹⁰R¹¹, —NR¹⁰COR¹¹, —OR¹⁰, or —SR¹⁰, wherein each occurrence of R¹⁰ is independently hydrogen, a protecting group, or an aliphatic, heteroaliphatic, aryl, heteroaryl, -(aliphatic)aryl, -(aliphatic)heteroaryl, -(heteroaliphatic)aryl, or -(heteroaliphatic)heteroaryl moiety,
whereby each of the foregoing aliphatic and heteroaliphatic moieties is substituted or unsubstituted, cyclic or acyclic, linear or branched and each of the foregoing cycloalipahtic, heterocycloaliphatic, aryl or heteroaryl moieties is independently substituted or unsubstituted.

40. The library of claim 39, wherein R¹ and R² represent one of the following structures: 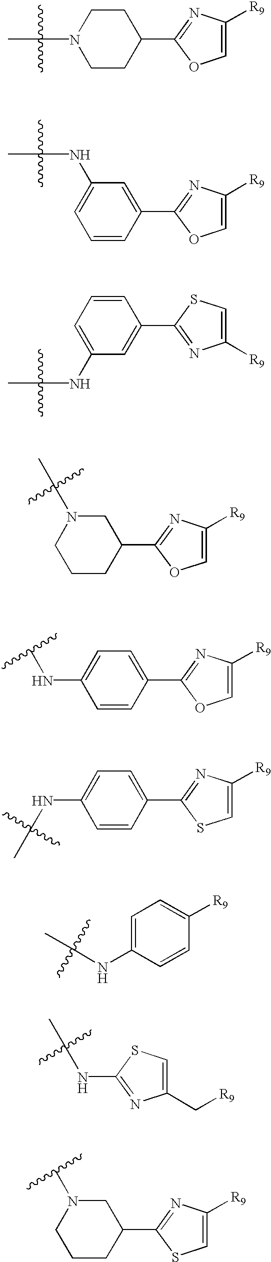
wherein R⁹ is COOH or is CO(NR¹⁰R¹¹), wherein each occurrence of R¹⁰ and R¹¹ is tit independently hydrogen, a protecting group, or an aliphatic, heteroaliphatic, aryl, heteroaryl, -(aliphatic)aryl, -(aliphatic)heteroaryl, -(heteroaliphatic)aryl, or -(heteroaliphatic)heteroaryl, whereby each of the foregoing aliphatic and heteroaliphatic moieties is substituted or unsubstituted, cyclic or acyclic, linear or branched and each of the foregoing cycloalipalitic, heterocycloaliphatic, aryl or heteroaryl moieties is independently substituted or unsubstituted.

41. The library of claim 21, wherein the library comprises at least 5 members.

42. The library of claim 21, wherein the library comprises at least 20 members.

43. The library of claim 21, wherein the library comprises at least 100 members.

44. The library of claim 21, wherein the library comprises at least 500 members.

45. The library of claim 21, wherein the library comprises at least 1000 members.

46. The library of claim 21, wherein each member has a different molecular weight.

47. The library of claim 21, wherein each member has a mass that differs from another member by at least 5 atomic mass units.

48. The library of claim 21, wherein each member has a mass that differs from another member by at least 10 atomic mass units.

49. A method for ligand discovery comprising:
contacting a target that comprises a chemically reactive group at or near a site of interest with a compound of claim 1 that is capable of forming a covalent bond with a chemically reactive group;
forming a covalent bond between the target and the compound thereby forming a target-compound conjugate; and
identifying the target compound conjugate.
